SEQUENCE LISTING (21 sequences)

SEQ ID NO: 1 - protein, 421 aa, Homo sapiens
MASSSVPPATVSAATAGPGPGFGFASKTKKKHFVQQKVKVFRAADPLVGVFLWGVAHSINELSQVPPPVMLLPDDFKASSKIKVNNHLFHRENLPSHFKFKEYCPQVFRNLRDRFGIDDQDYLVSLTRNPPSESEGSDGRFLISYDRTLVIKEVSSEDIADMHSNLSNYHQYIVKCHGNTLLPQFLGMYRVSVDNEDSYMLVMRNMFSHRLPVHRKYDLKGSLVSREASDKEKVKELPTLKDMDFLNKNQKVYIGEEEKKIFLEKLKRDVEFLVQLKIMDYSLLLGIHDIIRGSEPEEEAPVREDESEVDGDCSLTGPPALVGSYGTSPEGIGGYIHSHRPLGPGEFESFIDVYAIRSAEGAPQKEVYFMGLIDILTQYDAKKKAAHAAKTVKHGAGAEISTVHPEQYAKRFLDFITNIFA

SEQ ID NO: 2 - DNA, 1558 bp, Homo sapiens
gtccgctgtccggcctccggtcacgtgacagcagcgcaggtgagcgccgcttccggggtcgggcgcctggatagctgccggctccggcttccacttggtcggttgcgcgggagactatggcgtcctcctcggtcccaccagccacggtatcggcggcgacagcaggccccggcccaggtttcggcttcgcctccaagaccaagaagaagcatttcgtgcagcagaaggtgaaggtgttccgggcggccgacccgctggtgggtgtgttcctgtggggcgtagcccactcgatcaatgagctcagccaggtgcctcccccggtgatgctgctgccagatgactttaaggccagctccaagatcaaggtcaacaatcaccttttccacagggaaaatctgcccagtcatttcaagttcaaggagtattgtccccaggtcttcaggaacctccgtgatcgatttggcattgatgaccaagattacttgtacattgtgaagtgccatggcaacacgcttctgccccagttcctggggatgtaccgagtcagtgtggacaacgaagacagctacatgcttgtgatgcgcaatatgtttagccaccgtcttcctgtgcacaggaagtatgacctcaagggttccctagtgtcccgggaagccagcgataaggaaaaggttaaagaattgcccacccttaaggatatggactttctcaacaagaaccagaaagtatatattggtgaagaggagaagaaaatatttctggagaagctgaagagagatgtggagtttctagtgcagctgaagatcatggactacagccttctgctaggcatccacgacatcattcggggctctgaaccagaggaggaagcgcccgtgcgggaggatgagtcagaggtggatggggactgcagcctgactggacctcctgctctggtgggctcctatggcacctccccagagggtatcggaggctacatccattcccatcggcccctgggcccaggagagtttgagtccttcattgatgtctatgccatccggagtgctgaaggagccccccagaaggaggtctacttcatgggcctcattgatatccttacacagtatgatgccaagaagaaagcagctcatgcagccaaaactgtcaagcatggggctggggcagagatctctactgtccatccggagcagtatgctaagcgattcctggattttattaccaacatctttgcctaagagactgcctggttctctctgatgttcaaggtggtggggttctgagacacttgggggaattgtggggatattctagccaccagttctcttcttcctttgctaaattcaggctgcaggctccttccatccagataactccatcctgtcgagtaggctctttctgaccctcagaaatacattgtcctttttcctctttgcccatttttcttccctctcttcctccccatgagaagtctgcttgtagtattagaatgttattgttgactctctcccaagtgccttgatctttgtaatatctcctgttgtttctatgatatagg

SEQ ID NO: 3 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 4 - protein, 64 aa, Homo sapiens
MCNTNMSVPTDGAVTTSQIPASEQETLVRPKPLLLKLLKSVGAQKDTYTMKEFATKHRAKNIPV

SEQ ID NO: 5 - protein, 60 aa, Homo sapiens
MCNTNMSVPTDGAVTTSQIPASEQETLVRPKPLLLKLLKSVGAQKDTYTMKENHRTQVHL

SEQ ID NO: 6 - protein, 73 aa, Homo sapiens [sequence omitted]

SEQ ID NO: 7 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 8 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 9 - protein, 298 aa, Homo sapiens
MGPKDSAKCLHRGPQPSHWAAGDGPTQERCGPRSLGSPVLGLDTCRAWDHVDGQILGQLRPLTEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDPWEEPEDAAPVAPSVPASGYPELPTPRREVQSESAQEPGGVSPAEAQRAWWVLEPPGARDVEAQLRRLQEERTCKVCLDRAVSIVFVPCGHLVCAECAPGLQLCPICRAPVRSRVRTFLS

SEQ ID NO: 10 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 11 - protein, 172 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRVYTLKERCLQVVRSLVKPENYRRLDIVRSLYEDLEDHPNVQKDLERLTQERIAHQRMGD

SEQ ID NO: 12 - protein, 193 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRVLMTPVGQFCVVPALVENTFLLGRLTDAKTGTSQGHVGAGRADRVWRGKLTYLPAGRWRGCGCVVSVKEHFPEKEESRME

SEQ ID NO: 13 - protein, 442 aa, Homo sapiens [sequence omitted]

SEQ ID NO: 14 - protein, 441 aa, Homo sapiens
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 15 - protein, 379 aa, Homo sapiens
MEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 16 - protein, 398 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFRYAWTVAQCKICASHIGWKFTATKKDMSPQKFWGLTRSALLPTIPDTEDEISPDKVILCL

SEQ ID NO: 17 - protein, 475 aa, Homo sapiens [sequence omitted]

SEQ ID NO: 18 - DNA, 22 bp, Artificial Sequence
  A synthetic sequence
ttgaacatcagagagaaccagg

SEQ ID NO: 19 - DNA, 22 bp, Artificial Sequence
  A synthetic sequence
ttctaaagagcaatggttgctg

SEQ ID NO: 20 - DNA, 22 bp, Artificial Sequence
  A synthetic sequence
tattattatagtaacaggagca

SEQ ID NO: 21 - DNA, 23 bp, Artificial Sequence
  A synthetic sequence
atctggcagcagcatcaccgggg

(19) United States
(12) Patent Application Publication
(10) Pub. No.: US 2024/0167041 A1
(43) Pub. Date: May 23, 2024

(54) LOSS OF LIPID KINASE PI5P4K GAMMA RESTRICTS TUMOR GROWTH

(71) Applicants: Lewis C. Cantley, New York, NY (US); Vijay Kumar Kuchroo, Chestnut Hill, MA (US); Markus Alexander Schramm, Brookline, MA (US); Karen Olivia Dixon, Brookline, MA (US); Cornell University, Ithaca, NY (US); The Brigham and Women's Hospital, Inc., Boston, MA (US)

(72) Inventors: Lewis C. Cantley, New York, NY (US); Vijay Kumar Kuchroo, Chestnut Hill, MA (US); Markus Alexander Schramm, Brookline, MA (US); Karen Olivia Dixon, Brookline, MA (US)

(21) Appl. No.: 18/281,526

(22) PCT Filed: Mar. 11, 2022
(86) PCT No.: PCT/US2022/019908

§ 371 Date: Sep. 11, 2023

Related U.S. Application Data
(60) Provisional application No. 63/160,124, filed on Mar. 12, 2021.

(51) Int. Cl. C12N 15/113; A61K 35/15; A61K 35/17; A61K 35/18; A61K 35/28; A61P 35/00; C07K 16/40; C12N 5/09

(52) U.S. Cl. CPC C12N 15/1137; A61K 35/15; A61K 35/17; A61K 35/18; A61K 35/28; A61P 35/00; C07K 16/40; C12N 5/0693; C12Y 207/01149; C12N 2310/14; C12N 2310/531; C12N 2510/00

(57) ABSTRACT

Described herein are compositions and methods for inhibiting, degrading, knocking down or knocking out pip4k2c nucleic acids or Pip4k2c protein. Such compositions and methods are useful for treating and inhibiting the onset of cancer.

This application claims benefit of priority to the filing date of U.S. Provisional Application Ser. No. 63/160,124, filed Mar. 12, 2021, the contents of which are specifically incorporated by reference herein in their entirety.

### GOVERNMENT FUNDING

This invention was made with government support under PO1AI073748 awarded by the National Institutes of Health. The government has certain rights in the invention.

### INCORPORATION BY REFERENCE OF SEQUENCE LISTING PROVIDED AS A TEXT FILE

A Sequence Listing is provided herewith as a text file, “ 2221415.txt,” created on Mar. 8, 2022 and having a size of 45,056 bytes. The contents of the text file are incorporated by reference herein in their entirety.

### BACKGROUND

With more than 3.7 million new cases and 1.9 million deaths each year, cancer represents a significant cause of death and morbidity worldwide with the estimated direct health cost increasing from 87 to 95 billion during 2005-2014. However, the emerging field of immune-checkpoint therapy (ICT) has demonstrated unprecedented responses in patients with several types of metastatic tumors that were otherwise refractory to available treatment options. But recent studies show that across all indications only about 13% of patients respond to immunotherapy. Therefore, despite the promising potential of immune-checkpoint therapy, the majority of patients fail to respond, and most eventually their disease progresses. This is at least partially explained by the observation that the T cells in the tumor, which undergo T cell exhaustion/dysfunction, not only express CTLA4 or PD-1, but also express a module of co-inhibitory molecules, including PD-1, Tim-3, Lag3, TIGIT, and others. While combinational therapy may help to improve efficacy to immune-checkpoint therapy, T cell directed therapies often quickly reach a ceiling beyond which the T cell directed therapies will not work. Discovery of novel therapeutic targets is needed.

### SUMMARY

As illustrated herein, loss of Pip4k2c globally, for example using knockout alleles, degraders, inhibitory nucleic acids, antibodies, or other agents, leads to profound tumor control in vivo. During the course of disease mice lacking Pip4k2c expression (Pip4k2c^{-/-} mice) exhibited significantly retarded tumor growth. Such Pip4k2c^{-/-} mice even exhibited substantial tumor regression with generation of specific memory responses leading to accelerated tumor rejection upon re-challenge with parental tumor cell lines. As shown herein inhibition or deletion of Pip4k2c in myeloid populations, specifically dendritic cells, and regulatory T cells led to profound tumor control in mice.

Compositions and methods are therefore described herein for inhibiting, degrading, knocking down, or knocking out Pip4k2c nucleic acids and/or Pip4k2c proteins. Such compositions and methods are useful for treating and inhibiting the onset or progression of cancer.

Described herein are compositions that include one or more agents that can modify or inhibit Pip4k2c protein or a pip4k2c nucleic acid. Examples or such agents include one or more anti-Pip4k2c antibodies, Pip4k2c nucleic acid inhibitors, Pip4k2c degraders, Pip4k2c vaccines, small molecule Pip4k2c inhibitors, Pip4k2c guide RNAs with cas nucleases, and combinations thereof. Also described herein are populations of modified myeloid cells with knockdown or knockout of the cells' endogenous pip4k2c.

Methods are described herein that involve administering the compositions and/or population of modified myeloid cells to a subject. The cells can be myeloid cells, lymphocytes, regulatory T cells, dendritic cells, bone marrow cells, granulocytes, basophils, eosinophils, neutrophils, monocytes, mast cells, erythrocytes, macrophages, platelets, and combinations thereof. In some cases, such a subject can have cancer or be suspected of having or developing cancer.

Methods and kits are also described herein that can modify cells (e.g. myeloid cells) in vitro or in vivo to reduce Pip4k2c expression or function.

### DESCRIPTION OF THE FIGURES

FIG. 1A-1C illustrate that Pip4k2c^{-/-} mice exhibit profoundly decreased tumor growth compared to wild type Pip4k2c^{+/+} mice, even when different types of tumor cells are transplanted into the mice. FIG. 1A shows the tumor sizes of highly metastatic melanoma B16F10 and B 16F10-OVA cells that were transplanted into wild type Pip4k2c^{+/+} mice (circle symbols) and Pip4k2c^{+/+} mice (square symbols). FIG. 1B shows the tumor sizes of colon adenocarcinoma MC38 cells that were transplanted into wild type Pip4k2c^{+/+} mice (circle symbols) and Pip4k2c^{-/-} mice (square symbols). FIG. 1C graphically illustrates the percent of total lung area of KP1.9 lung adenocarcinoma cells in wild type Pip4k2c^{+/+} mice (left bar) and knockout Pip4k2c^{-/-} mice (right bar).

FIG. 2A-2C illustrate that Pip4k2c^{-/-} mice develop profound memory responses. FIG. 2A graphically illustrates tumor sizes of Pip4k2c^{+/+} mice (circles) and Pip4k2c^{-/-} mice (squares) over time after administration of MC38OVA colon adenocarcinoma cells. As illustrated, the Pip4k2c^{-/-} mice cleared the tumors, but the wild type mice do not. FIG. 2B graphically illustrates tumor sizes in the same mice one month later after re-challenge of Pip4k2c^{-/-} mice (squares) and Pip4k2c+/+ wild type control mice (circles) with the parental cell-MC38 tumor cells that did not express OVA. As illustrated, the Pip4k2c^{-/-} mice showed profound accelerated rejection of the parental tumor indicating that memory T cells had formed during the one month since the initial tumors receded in the Pip4k2c^{-/-} mice. FIG. 2C graphically illustrates that the survival of the Pip4k2c^{-/-} mice (long dashed line at top) treated as described in FIG. 2B was significantly prolonged compared to wild type mice controls (solid line and dashed, dotted line).

FIG. 3A-3B illustrate that Pip4k2c^{-/-} mice have significantly fewer tumor foci than wild type mice controls. FIG. 3A shows images of lungs from wild type Pip4k2c^{+/+} mice (top row) and from Pip4k2c^{-/-} mice (bottom row), showing that after intravenous administration of B16 melanoma cells, fewer tumor foci engraft within Pip4k2c^{-/-} lungs, than in the wild type Pip4k2c^{+/+} lungs. FIG. 3B graphically illustrates the numbers of tumor foci in the lungs of wild type Pip4k2c^{+/+} mice (left bar) and in the Pip4k2c^{-/-} mice (right bar).

FIG. 4A-4B illustrate that tumor growth is driven by hemopoietic cells. FIG. 4A shows images of B16 melanoma tumors from irradiated mice with reconstituted bone marrow from either wild type or Pip4k2c^{-/-} mice. Mice receiving wild type bone marrow (top row) had larger tumors than mice receiving Pip4k2c^{-/-} bone marrow (bottom row). FIG. 4B graphically illustrates the weights of tumors from mice receiving wild type bone marrow (left bar) compared to mice receiving Pip4k2c^{-/-} bone marrow (right bar). FIG. 4C graphically illustrates the sizes of tumors from mice receiving wild type bone marrow (circular symbols) compared to mice receiving Pip4k2c^{-/-} bone marrow (square symbols). In addition to illustrating that administration of Pip4k2c^{-/-} bone marrow reduces tumor burden, these results show that the tumor phenotype is driven by hematopoietic cells. The results shown are from one experiment, representative of two independent experiments. *P<0.05; **P<0.01 ***P<0.001 ****P<0.0001 (Student Two-Tailed T test)

FIG. 5A-5B illustrate that depletion of either CD8 T cells or natural killer (NK) cells ablates protective effect of Pip4k2c deficiency. FIG. 5A graphically illustrates tumor sizes of wild type and Pip4k2c^{-/-} mice with (aCD8) and without treatment of antibodies to deplete CD8 T cells. As shown, tumor sizes in Pip4k2c^{-/-} mice are much smaller when no antibodies are used to deplete CD8 T cells (lower line with squares) than were the tumor sizes in Pip4k2c^{-/-} mice when antibodies are used to deplete CD8 T cells (aCD8, upper lines with triangle symbols). FIG. 5B graphically illustrates tumor sizes of wild type and Pip4k2c^{-/-} mice with (aNK1.1) and without treatment of antibodies to deplete natural killer cells. As shown, tumor sizes in Pip4k2c^{-/-} mice are much smaller when no antibodies are used to deplete natural killer cells (lower line with squares) than the tumor sizes in Pip4k2c^{-/-} mice when antibodies are used to deplete natural killer cells (aNK1.1, upper lines with triangle symbols).

FIG. 6A-6G illustrate that global deficiency of Pip4k2c leads to dramatically increased immune cell infiltration. FIG. 6A graphically illustrates the total numbers of

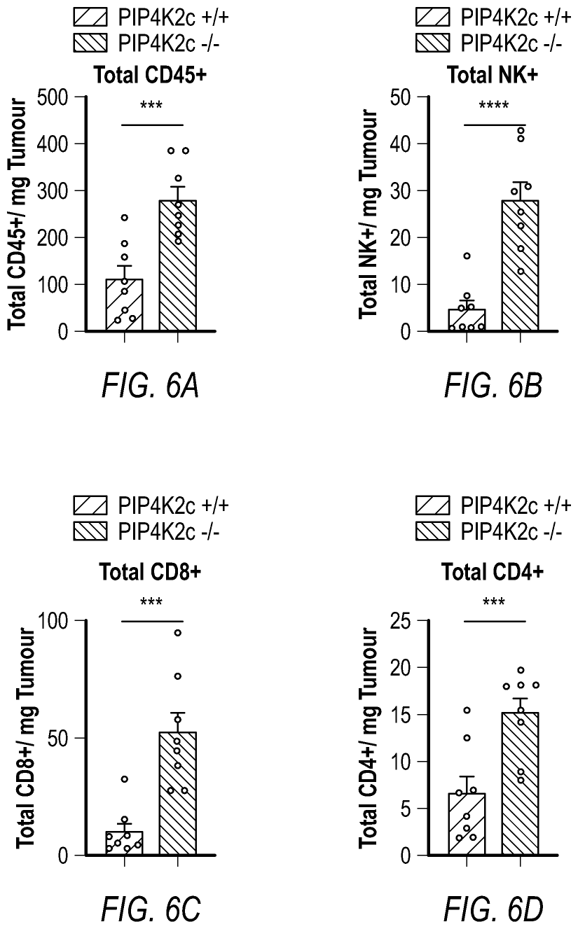

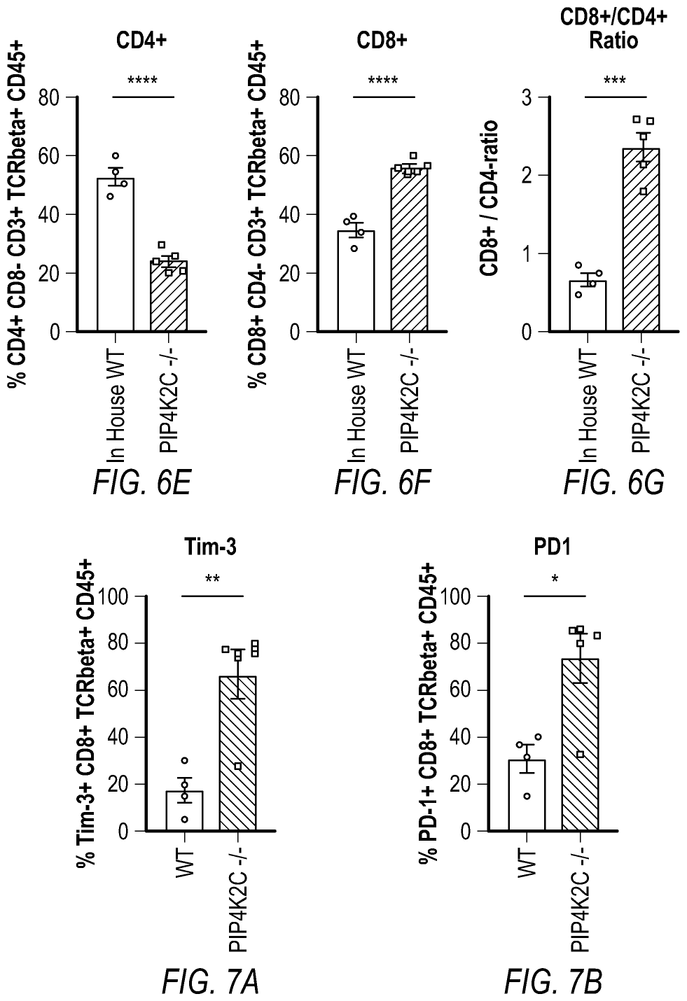

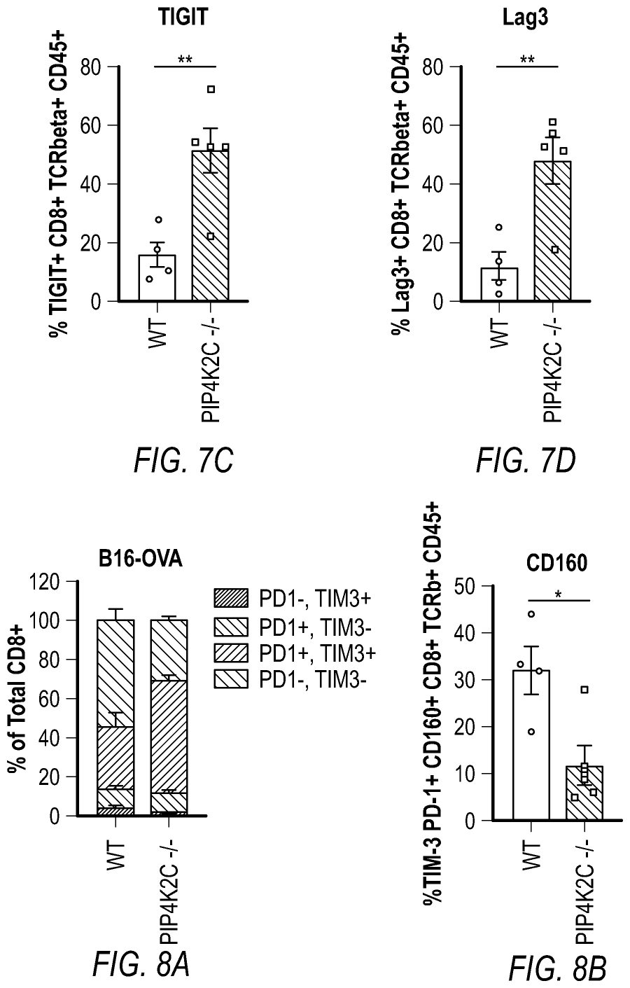

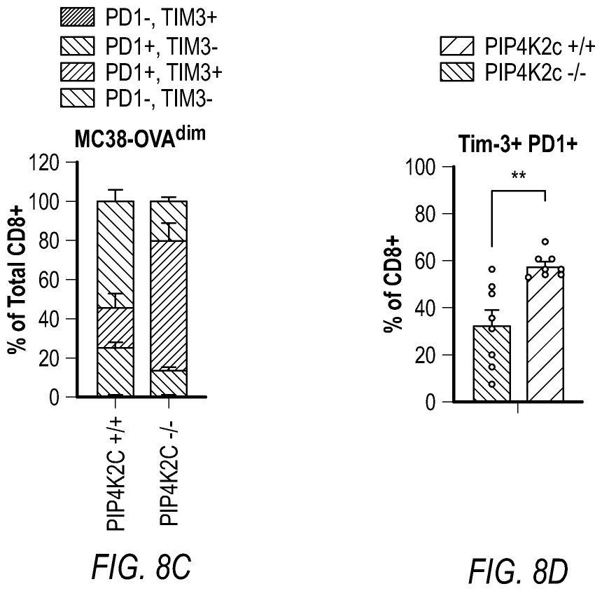

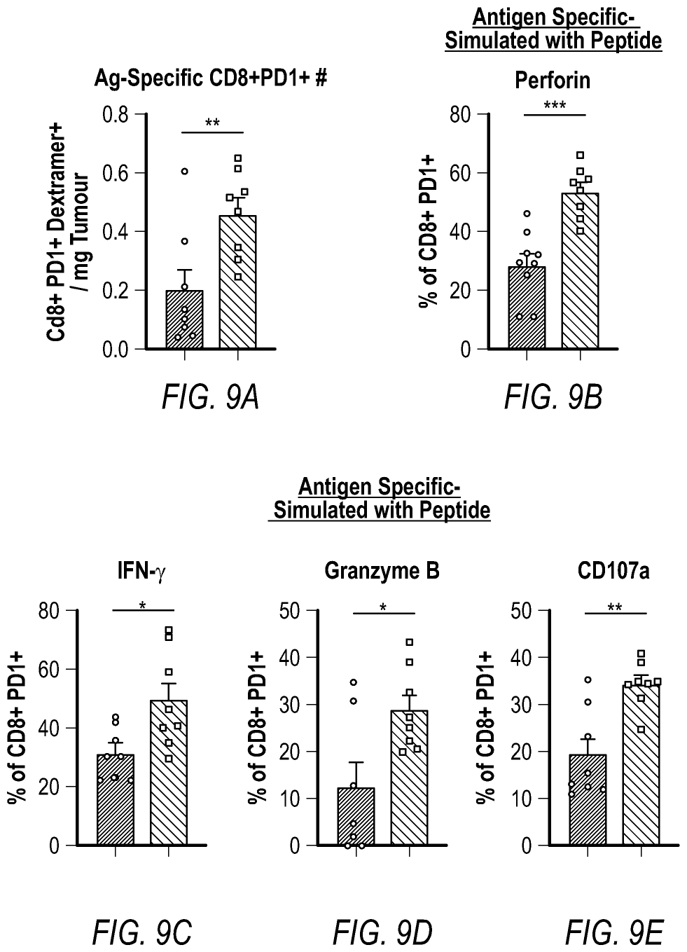

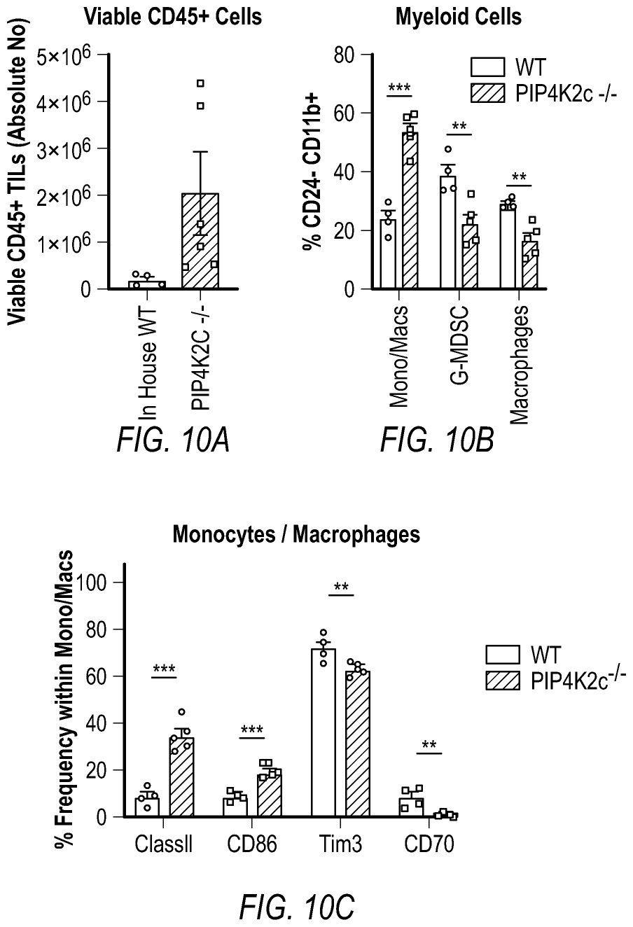

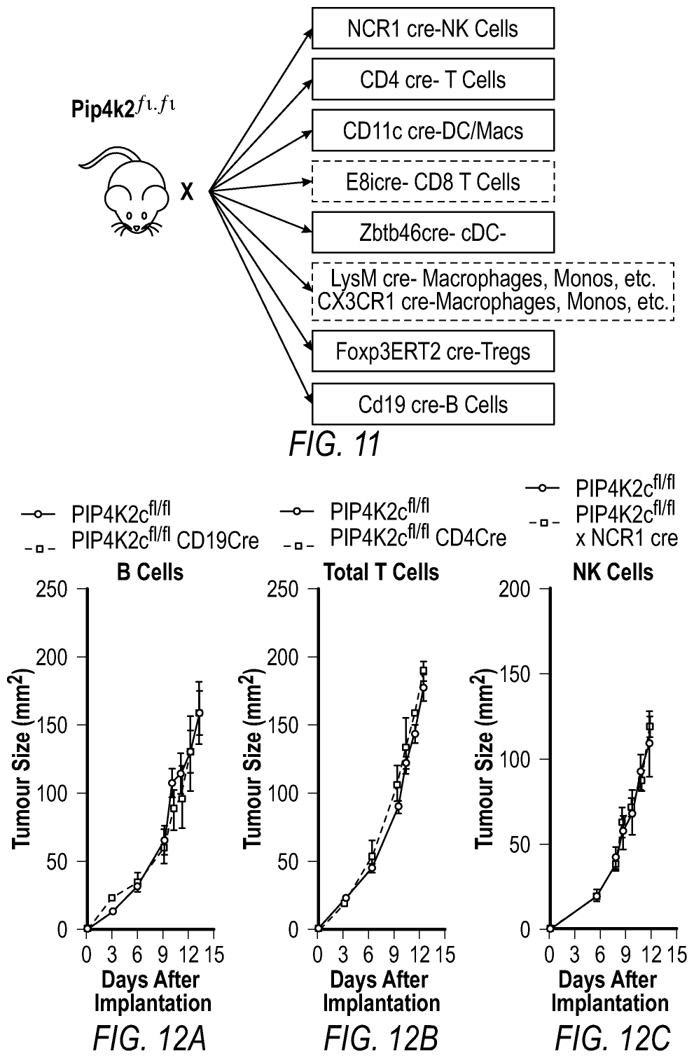

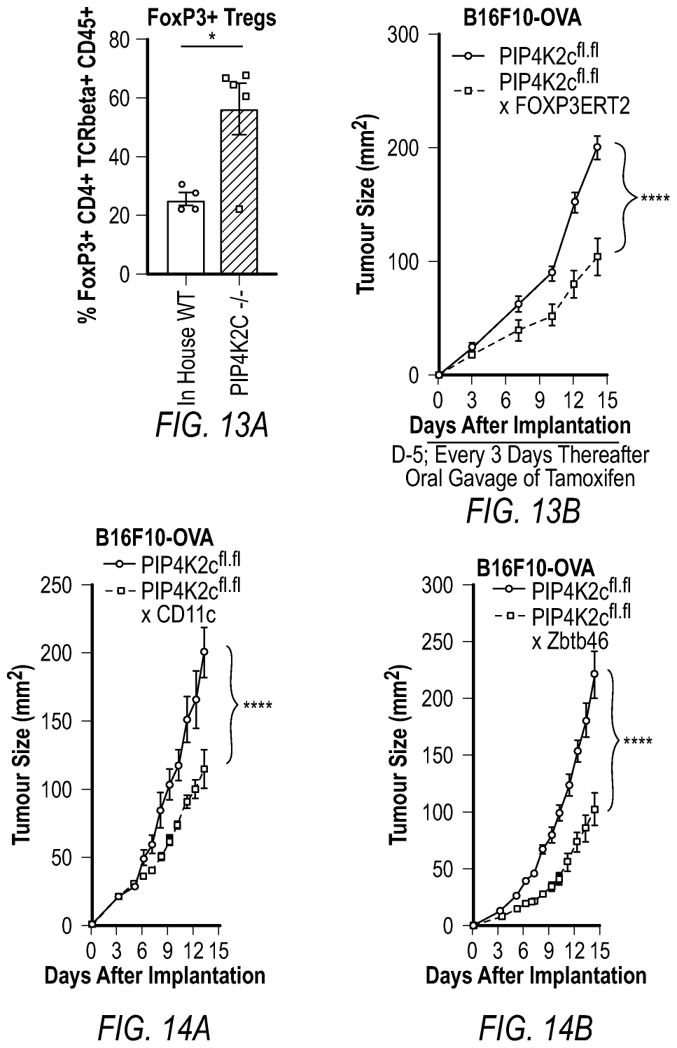

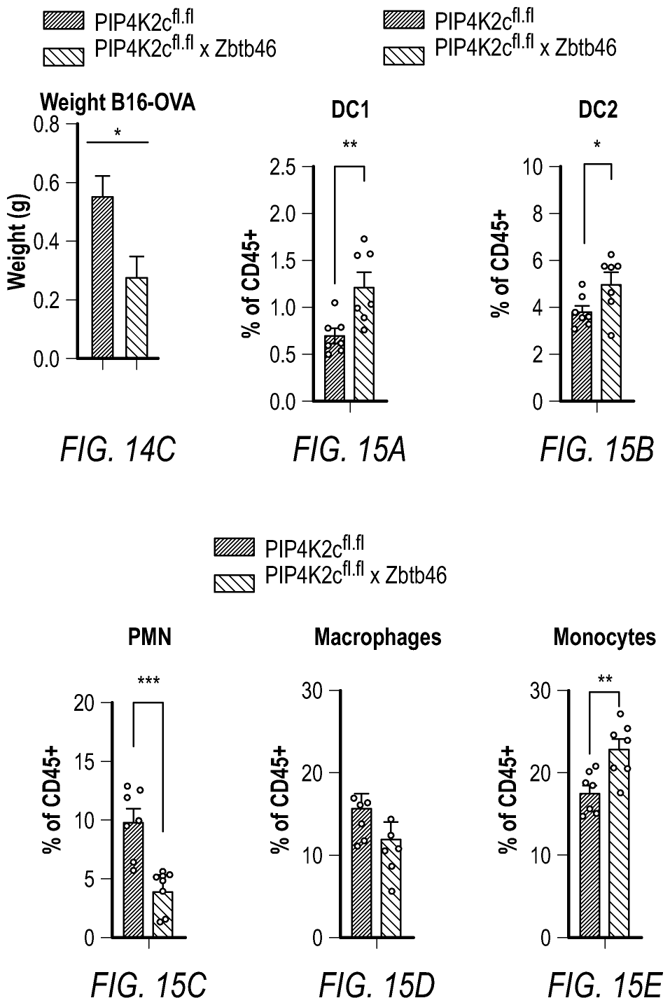

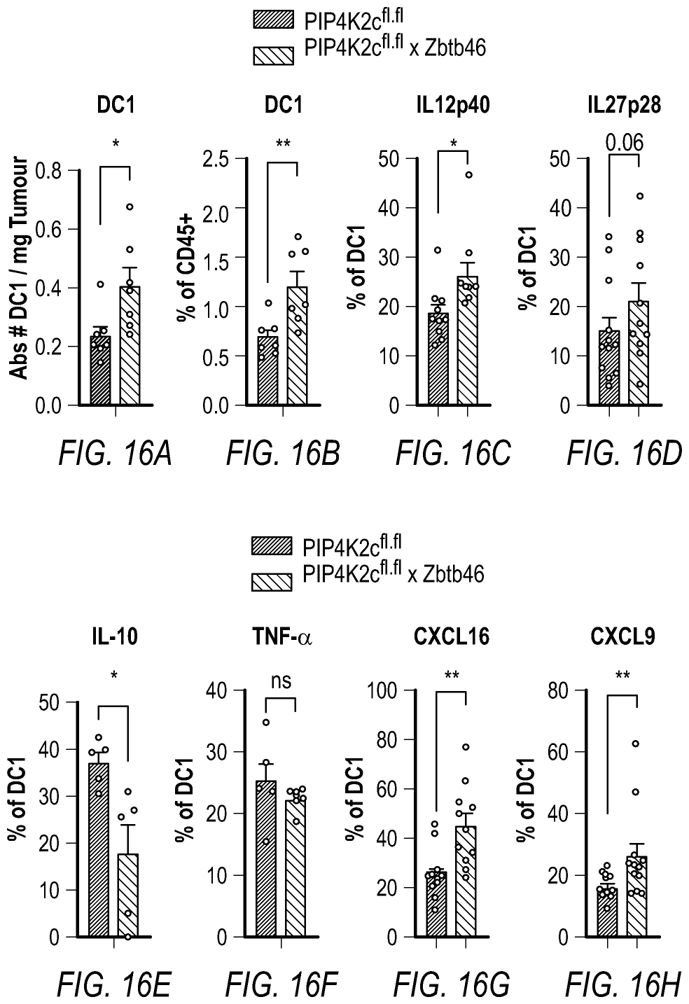

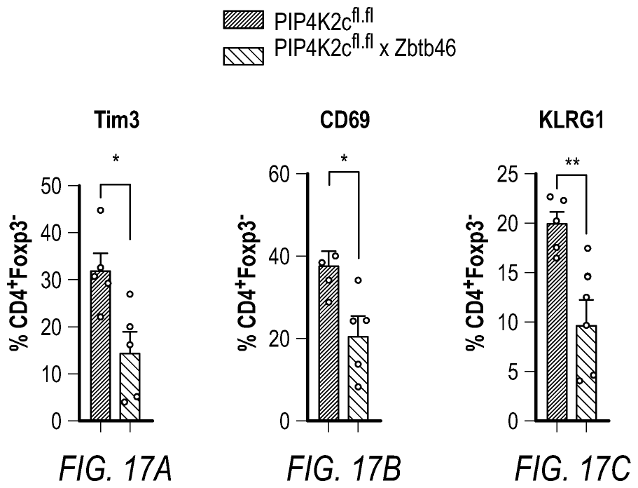

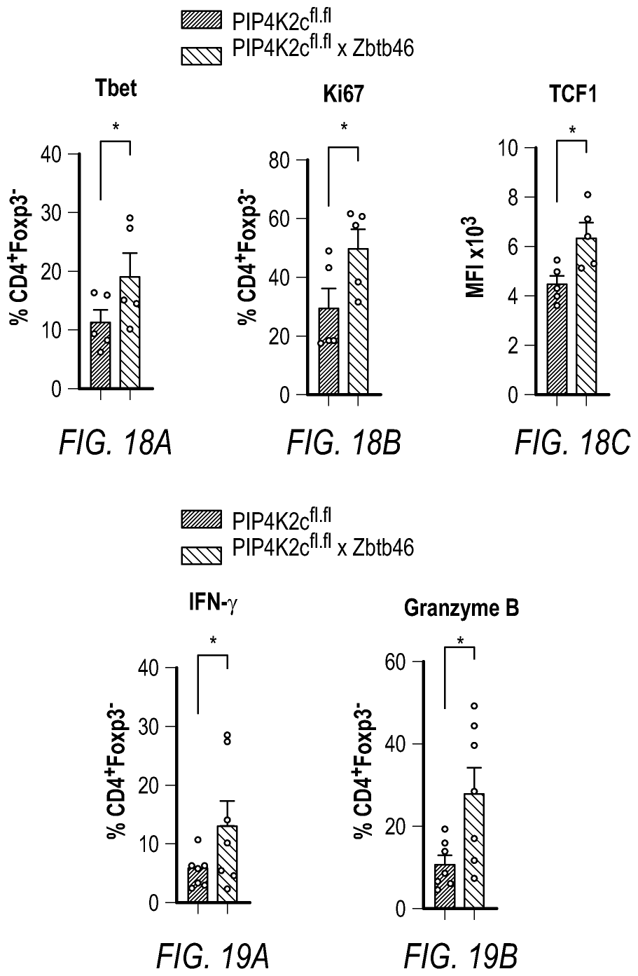

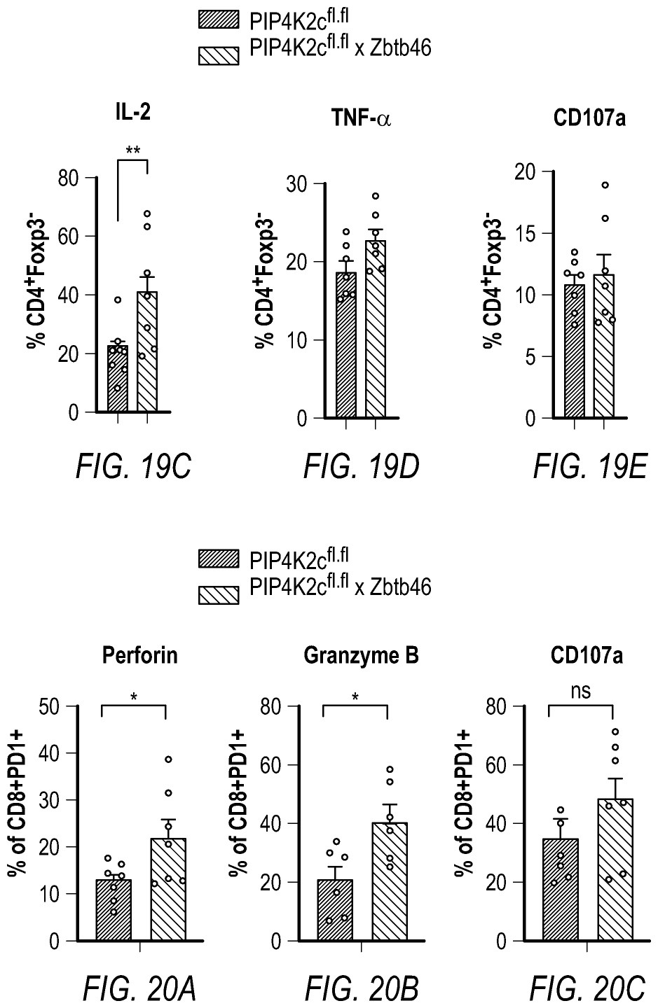

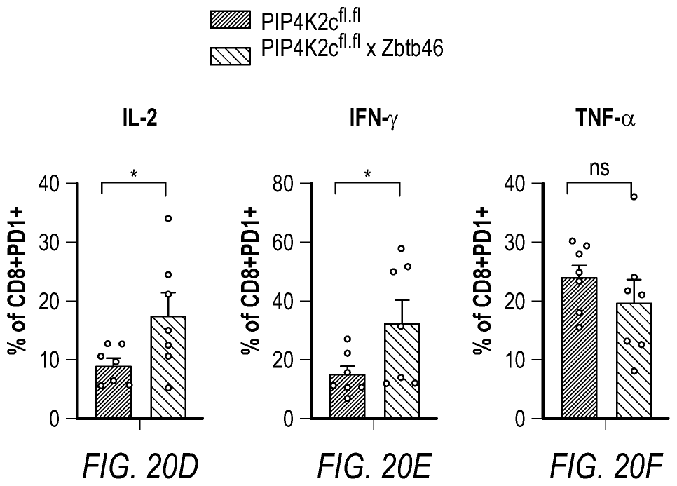

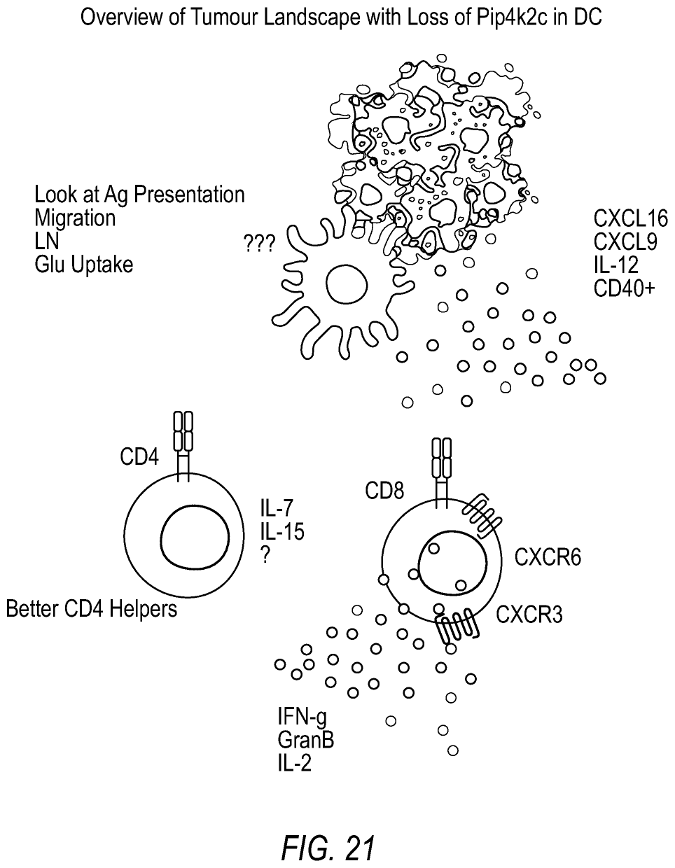

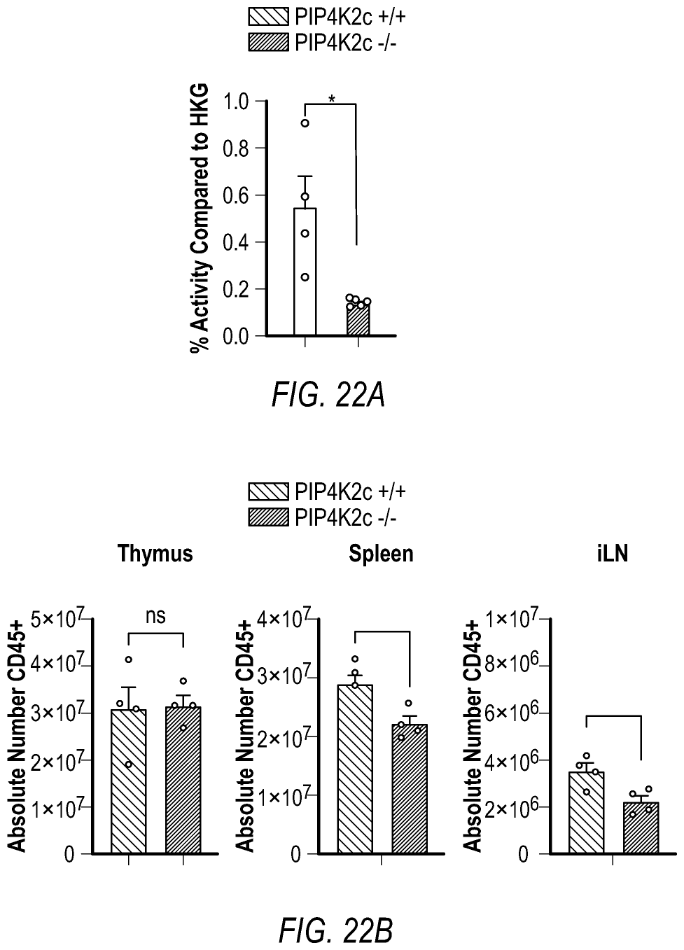

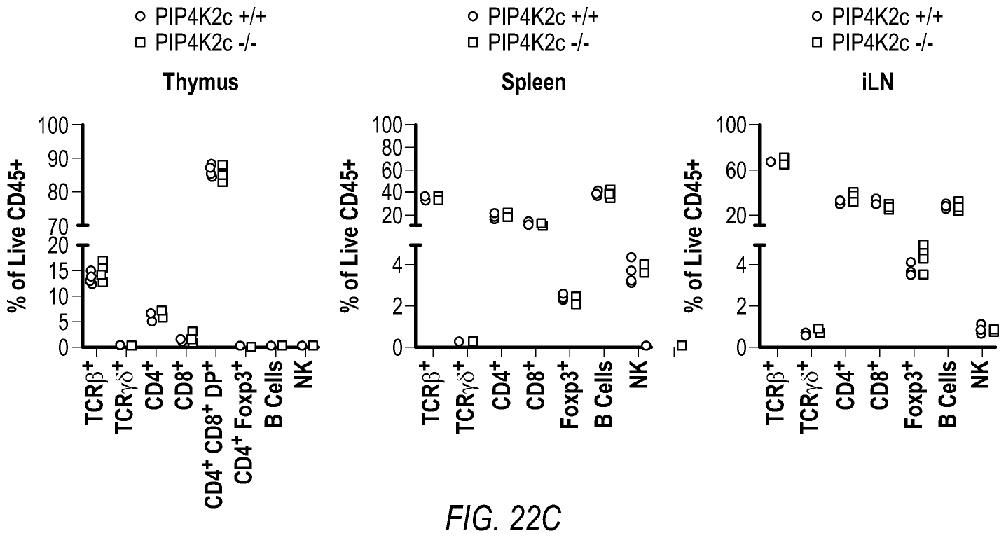

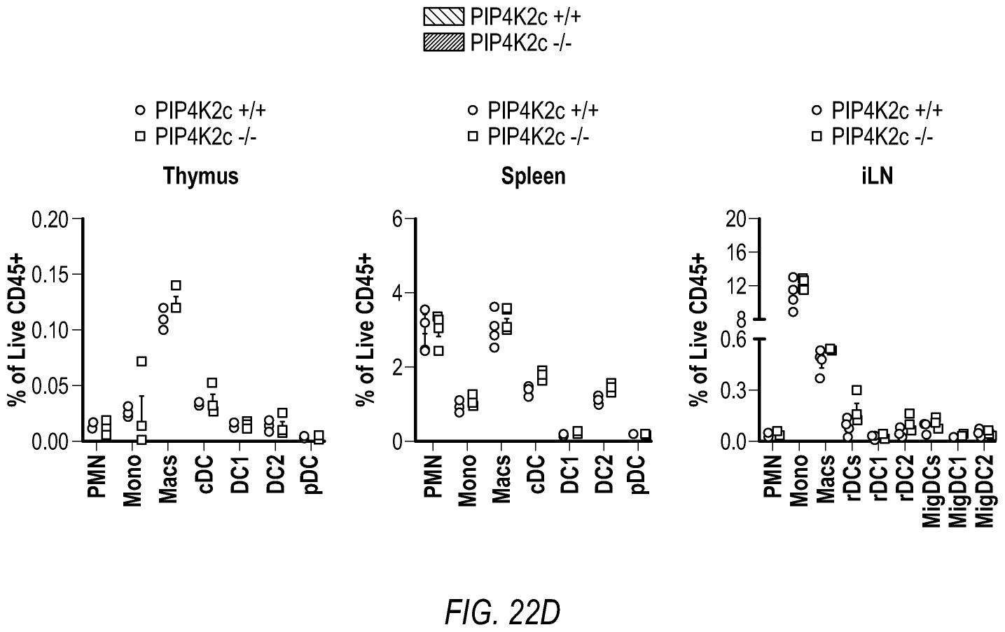

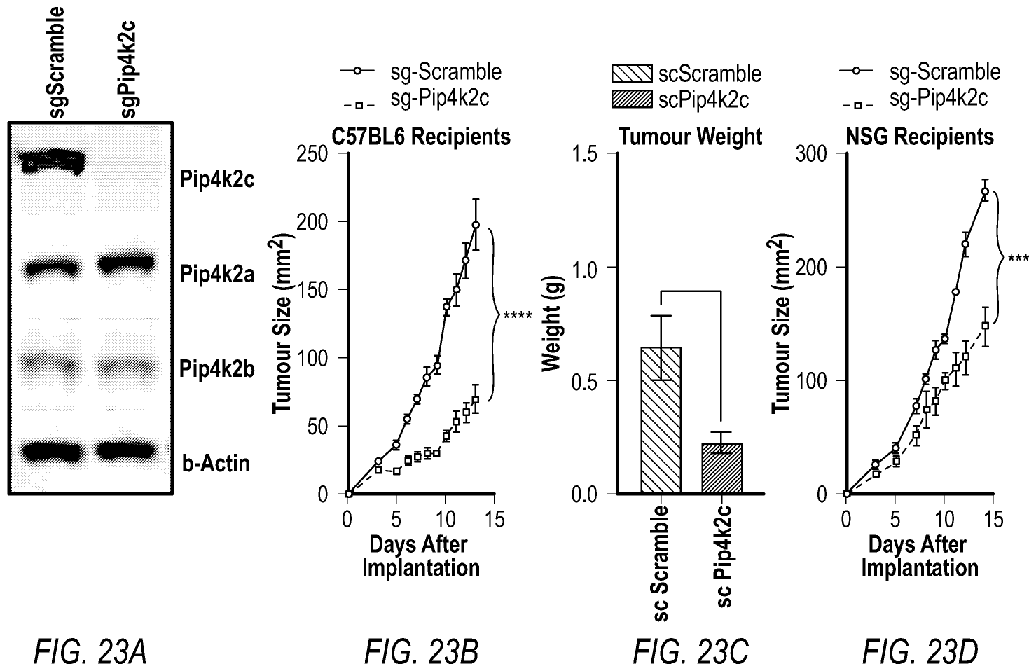

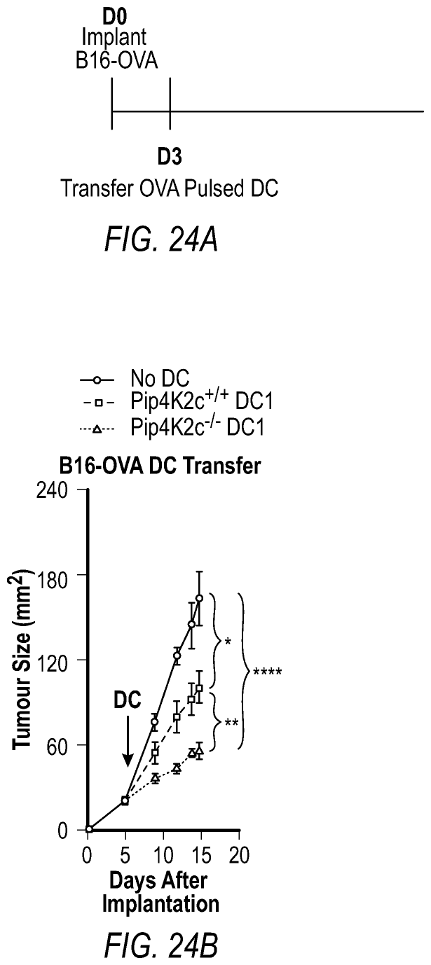

CD45+ cells per mg of tumor in wild type and Pip4k2c^{-/-} mice. FIG. 6B graphically illustrates the total numbers of natural killer cells per mg of tumor in wild type and Pip4k2c^{-/-} mice. FIG. 6C graphically illustrates the total numbers of CD8+ cells per mg of tumor in wild type and Pip4k2c^{-/-} mice. FIG. 6D graphically illustrates the total frequency of CD4+ cells in wild type and Pip4k2c^{-/-} tumor-bearing mice. FIG. 6E illustrates that CD4+ cell frequencies are reduced in tumors within Pip4k2c^{-/-} mice compared to CD4+ cell frequencies in tumors within wild type mice. FIG. 6F illustrates that CD8+ cell frequencies are increased in tumors within Pip4k2c^{-/-} mice compared to CD8+ cell frequencies in tumors within wild type mice. FIG. 6G graphically illustrates the ratio of CD8⁺/CD4+ cells in tumors within wild type and Pip4k2c^{-/-} mice. As shown there is a significantly increased ratio of CD8:CD4 T cells in Pip4k2c^{-/-} mice. For FIG. 6A-6G, the numbers of these immune cells in tumors of Pip4k2c^{-/-} mice are shown in the bars to the right, while the numbers of these immune cells in tumors of wild type mice are shown in the bars to the left.

FIG. 7A-7D illustrate that CD8⁺ tumor infiltrating lymphocytes (TILs) in Pip4k2c^{-/-} mice express high levels of T cell activation markers. FIG. 7A graphically illustrates increased levels of Tim-3 in CD8+ TILs. FIG. 7B graphically illustrates increased levels of PD1 in CD8⁺ TILs. FIG. 7C graphically illustrates increased levels of TIGIT in CD8⁺ TILs. FIG. 7D graphically illustrates increased levels of Lag3 in CD8+ TILs. For FIG. 7A-7D, the percentages of immune cells in tumors of Pip4k2c^{-/-} mice are shown in the bars to the right, while the numbers of these immune cells in tumors of wild type mice are shown in the bars to the left. These data illustrate that CD8⁺ T cells from tumors of Pip4k2c^{-/-} mice are highly activated.

FIG. 8A-8D illustrate that Tim-3⁺ PD-1⁺ CD8⁺ tumor infiltrating lymphocytes (TILs) in Pip4k2c^{-/-} mice are less exhausted. FIG. 8A graphically illustrates the proportions of CD8⁺ T cells that express PD1 and Tim3 in B16-OVA tumors from wild type and Pip4k2c^{-/-} mice at 14 days after B16-OVA tumor cell administration. FIG. 8B graphically illustrates expression levels of the exhaustion marker CD160 in CD8⁺ T cells from B16-OVA tumors of wild type and Pip4k2c^{-/-} mice. FIG. 8C graphically illustrates the proportions of CD8⁺ T cells that express PD1 and Tim3 in MC38-OVA tumors from wild type and Pip4k2c^{-/-} mice as detected by flow cytometric quantification of immune cells in tumours from Pip4k2c^{+/+} (WT) or Pip4k2c^{-/-} mice at 14 days after MC38-OVA tumor cell administration. FIG. 8D graphically illustrates the frequency of CD8⁺ Tim3⁺PD1⁺ T cells in B16-OVA tumors from wild type and Pip4k2c^{-/-} mice at 14 days after B16-OVA tumor cell administration.

FIG. 9A-9E illustrate that CD8⁺ antigen-specific tumor infiltrating lymphocytes from Pip4k2c^{-/-} tumors are highly cytolytic in response to stimulation with specific peptide antigens (e.g., OVA). FIG. 9A graphically illustrates the frequency of OVA-specific CD8⁺ PD-1⁺ T cells per mg tumor from Pip4k2c^{-/-} tumors compared to wild type tumors. FIG. 9B graphically illustrates that the OVA-specific PD-1⁺ CD8⁺ T cells from Pip4k2c^{-/-} tumors express higher levels of perforin than the PD-1⁺ CD8⁺ T cells from wild type tumors. FIG. 9C graphically illustrates that OVA-specific PD-1⁺ CD8⁺ T cells from Pip4k2c^{-/-} tumors (right bar) express higher levels of interferon-γ than PD-1⁺ CD8⁺ T cells from wild type tumors (left bar). FIG. 9D graphically illustrates that OVA-specific PD-1⁺ CD8⁺ T cells from Pip4k2c^{-/-} tumors (right bar) express higher levels of granzyme B than PD-1⁺ CD8⁺ T cells from wild type tumors (left bar). FIG. 9E graphically illustrates that OVA-specific PD-1⁺ CD8⁺

T cells from Pip4k2c^{-/-} tumors (right bar) express higher levels of CD107a than PD-1⁺ CD8+ T cells from wild type tumors (left bar).

FIG. 10A-10C illustrate that Pip4k2c knockout results in major changes within the myeloid compartment of mice. FIG. 10A shows that Pip4k2c^{-/-} tumors have increased numbers of viable CD45⁺ cells compared to wild type tumors. FIG. 10B shows that Pip4k2c^{-/-} tumors have increased percentages of CD24⁻ CD11b⁺ myeloid cells such as compared to wild type tumors. FIG. 10C shows that Pip4k2^{-/-} monocytes and macrophages express higher percentages of activation markers such as MHC class II molecules and CD86.

FIG. 11 is a schematic diagram illustrating crosses of a conditional Pip4k2c flox allele with different immune specific cre lines to deplete Pip4k2c in distinct cell types within mice. Rather than global Pip4k2c loss, different mouse lines were generated, specifically deleting Pip4k2c in a single immune cell type.

FIG. 12A-12C show that there is no significant tumor growth inhibition in mice with conditional deletions of Pip4k2c in total B cells, T cells or natural killer (NK) cells generated as illustrated in FIG. 11. FIG. 12A shows that tumor sizes are not reduced in animals with Pip4k2c^{-/-} B cells. FIG. 12B shows that tumor sizes are not reduced in animals with Pip4k2c^{-/-} total T cells. FIG. 12C shows that tumor sizes are not reduced in animals with Pip4k2c^{-/-} natural killer cells.

FIG. 13A-13B illustrate that there were increases FoxP3⁺ Tregs and reduced tumor sizes in tumor-bearing Pip4k2c^{-/-} mice. FIG. 13A graphically illustrates higher percentages of FoxP3⁺ Tregs were present in tumor-bearing Pip4k2c^{-/-} mice than in tumor-bearing wild type mice. FIG. 13B graphically illustrates reduced tumor sizes in tumor-bearing Pip4k2c^{-/-} mice (square symbols) that have a deletion of Pip4k2c only in regulatory T cells compared to tumor-bearing wild type mice (circular symbols).

FIG. 14A-14C illustrate that the most significant reduction in tumor sizes were in mice with conditionally Pip4k2c-deleted dendritic cells (Pip4k2c^{fl.fl}×CD11c and Pip4k2c^{fl.fl}×Zbtb46). FIG. 14A illustrates tumor sizes over time in mice with Pip4k2c-deleted dendritic cells (Pip4k2c^{fl.fl}×CD11c, square symbols) compared to mice with wild type Pip4k2c (circular symbols). FIG. 14B illustrates tumor sizes over time in mice with Pip4k2c-deleted dendritic cells (Pip4k2c^{fl.fl}×Zbtb46, square symbols) compared to mice with wild type Pip4k2c (circular symbols). FIG. 14C graphically illustrates the average weights of tumors in mice with Pip4k2c-deleted dendritic cells (Pip4k2c^{fl.fl}×Zbtb46; right bar) compared to mice with wild type Pip4k2c (left bar).

FIG. 15A-15E illustrate that deletion of Pip4k2c in dendritic cells leads to increased frequency of dendritic cells within tumors. FIG. 15A graphically illustrates that deletion of Pip4k2c in dendritic cells (right bar) increases the percentage of DC1 cells in the population of CD45+ cells relative to wild type dendritic cells (left bar). FIG. 15B graphically illustrates that deletion of Pip4k2c in dendritic cells (right bar) increases the percentage of DC2 cells in the population of CD45⁺ cells relative to wild type dendritic cells (left bar). FIG. 15C graphically illustrates that deletion of Pip4k2c in dendritic cells (right bar) reduces the percentage of polymorphonuclear leukocytes (PMNs) in the population of CD45⁺ cells relative to wild type dendritic cells (left bar). FIG. 15D graphically illustrates that deletion of Pip4k2c in dendritic cells (right bar) reduces the percentage of macrophages in the population of CD45⁺ cells relative to wild type dendritic cells (left bar). FIG. 15E graphically illustrates that deletion of Pip4k2c in dendritic cells (right bar) increases the percentage of monocytes in the population of CD45⁺ cells relative to wild type dendritic cells (left bar).

FIG. 16A-16H illustrate that significantly elevated levels of chemokines important for effector T cell migration in Pip4k2c deficient dendritic cells (DCs). FIG. 16A graphically illustrates the absolute number of DC1 cells per mg tumor in mice with Pip4k2c-deleted dendritic cells (right bar) compared to the number of DC1 cells per mg tumor in wild type mice (left bar). FIG. 16B graphically illustrates that deletion of Pip4k2c in dendritic cells (right bar) increases the percentage of DC1 cells in the population of CD45⁺ cells relative to wild type dendritic cells (left bar). FIG. 16C graphically illustrates the percent of DC1 cells that express IL12p40 in mice with Pip4k2c-deleted dendritic cells (right bar) compared to the percent of DC1 cells that express IL12p40 in wild type mice (left bar). FIG. 16D graphically illustrates the percent of DC1 cells that express IL27p28 in mice with Pip4k2c-deleted dendritic cells (right bar) compared to the percent of DC1 cells that express IL27p28 in wild type mice (left bar). FIG. 16E graphically illustrates the percent of DC1 cells that express IL-10 in mice with Pip4k2c-deleted dendritic cells (right bar) compared to the percent of DC1 cells that express IL-10 in wild type mice (left bar). FIG. 16F graphically illustrates the percent of DC1 cells that express TNF-alpha in mice with Pip4k2c-deleted dendritic cells (right bar) compared to the percent of DC1 cells that express TNF-alpha in wild type mice (left bar). FIG. 16G graphically illustrates the percent of DC1 cells that express CXCL16 in mice with Pip4k2c-deleted dendritic cells (right bar) compared to the percent of DC1 cells that express CXCL16 in wild type mice (left bar). FIG. 16H graphically illustrates the percent of DC1 cells that express CXCL9 in mice with Pip4k2c-deleted dendritic cells (right bar) compared to the percent of DC1 cells that express CXCL9 in wild type mice (left bar).

FIG. 17A-17C illustrate that that the CD4 T helper cells from mice with Pip4k2c-deleted dendritic cells are less exhausted and the CD4 compartment appears less terminally differentiated. FIG. 17A graphically illustrates the percent of CD4⁺Foxp3⁻ cells that express Tim3 from wild type mice (left bar) and from mice with Pip4k2c-deleted dendritic cells (right bar). FIG. 17B graphically illustrates the percent of CD4⁺Foxp3⁻ cells that express CD69 from wild type mice (left bar) and from mice with Pip4k2c-deleted dendritic cells (right bar). FIG. 17C graphically illustrates the percent of CD4⁺Foxp3⁻ cells that express KLRG1 from wild type mice (left bar) and from mice with Pip4k2c-deleted dendritic cells (right bar). The lower expression of PD1 and KLRG1 mice in CD4+Foxp3− cells from Pip4k2c-deleted dendritic cells indicates that the CD4 T helper cells are less exhausted.

FIG. 18A-18C illustrate that there are increased CD4 effector T cells in Pip4k2c-deleted tumors. FIG. 18A graphically illustrates that the number of CD4±Foxp3⁻ cells expressing Tbet was increased in Pip4k2c-deleted tumors (right bar) compared to wild tumors (left bar) after the different tumor cells were administered to mice. FIG. 18B graphically illustrates that the number of CD4±Foxp3⁻ cells expressing Ki67 was increased in Pip4k2c-deleted tumors (right bar) compared to wild tumors (left bar) after the different tumor cells were administered to mice. FIG. 18C graphically illustrates that the number of CD4⁺Foxp3⁻ cells expressing TCF1 was increased in Pip4k2c-deleted tumors (right bar) compared to wild tumors (left bar) after the different tumor cells were administered to mice.

FIG. 19A-19E graphically illustrate the phenotype of CD4 T cells isolated from wild type and Pip4k2c^{Δ}DC (Pip4k2c Zcre) tumor bearing mice. FIG. 19A shows that the percent of CD4⁺Foxp3⁻ cells expressing interferon-γ was increased in Pip4k2c-deleted tumors (right bar) compared to wild tumors (left bar) after the different tumor cells were administered to mice. FIG. 19B graphically illustrates that the percent of CD4⁺Foxp3⁻ cells expressing granzyme-B was increased in tumor-bearing Pip4k2c^{Δ}DC (right bar) compared to wildtype tumor bearing mice (left bar). FIG. 19C graphically illustrates that the percent of CD4⁺Foxp3⁻ cells expressing interleukin-2 was increased in Pip4k2c-deleted tumors (right bar) compared to wild tumors (left bar) after the different tumor cells were administered to mice. FIG. 19D graphically illustrates that the percent of CD4⁺Foxp3⁻0 cells expressing TNF-alpha was slightly (not significantly) increased in Pip4k2c-deleted tumors (right bar) compared to wild tumors (left bar) after the different tumor cells were administered to mice. FIG. 19E graphically illustrates that the percent of CD4⁺Foxp3⁻ cells expressing CD107a was about the same or slightly (not significantly) increased in Pip4k2c-deleted tumors (right bar) compared to wild tumors (left bar) after the different tumor cells were administered to mice.

FIG. 20A-20F graphically illustrates that deletion of Pip4k2c in dendritic cells induces more functional CD8+ CTL responses. FIG. 20A graphically illustrates that the percent of CD8⁺PD1⁺ cells expressing perforin was increased in Pip4k2c-deleted tumors (right bar) compared to wild tumors (left bar) after the different tumor cells were administered to mice. FIG. 20B graphically illustrates that the percent of CD8⁺PD1⁺ cells expressing granzyme B was increased in Pip4k2c-deleted tumors (right bar) compared to wild tumors (left bar) after the different tumor cells were administered to mice. FIG. 20C graphically illustrates that the percent of CD8⁺PD1⁺ cells expressing CD107a was increased in Pip4k2c-deleted tumors (right bar) compared to wild tumors (left bar) after the different tumor cells were administered to mice. FIG. 20D graphically illustrates that the percent of CD8⁺PD1⁺ cells expressing interleukin-2 was increased in Pip4k2c-deleted tumors (right bar) compared to wild tumors (left bar) after the different tumor cells were administered to mice. FIG. 20E graphically illustrates that the percent of CD8⁺PD1⁺ cells expressing interferon-y was increased in Pip4k2c-deleted tumors (right bar) compared to wild tumors (left bar) after the different tumor cells were administered to mice. FIG. 20F graphically illustrates that the percent of CD8⁺PD1⁺ cells expressing TNF-alpha was slightly decreased in Pip4k2c-deleted tumors (right bar) compared to wild tumors (left bar) after the different tumor cells were administered to mice.

FIG. 21 is a schematic diagram illustrating the tumor landscape when Pip4k2c is depleted (e.g., knocked out) from dendritic cells.

FIG. 22A-22D illustrates that Pip4k2c deficiency does not lead to significant changes in immune populations at homeostasis. FIG. 22A graphically illustrates that Pip4k2c^{-/-} mice are deficient for transcripts of Pip4k2c as detected by qPCR analysis relative to mean housekeeping gene (HKG) expression. FIG. 22B graphically illustrates the absolute number of total leukocytes in WT (Pip4k2c+/+) versus knockout (KO) Pip4k2c−/− mouse thymus, spleen and lymph nodes, as detected by flow cytometry analysis. FIG. 22C graphically illustrates the percentages of live CD45+ cells that are T cells, B cells and NK cells in thymus, spleen and lymph nodes from WT (Pip4k2c+/+) versus Knockout (Pip4k2c−/−) mice as detected by flow cytometry analysis. FIG. 22D illustrates the percentages of live CD45+ cells that are various types of myeloid cells including neutrophils, monocytes, macrophages and DCs in thymus, spleen and lymph nodes of WT (Pip4k2c+/+) versus KO (Pip4k2c−/−) mice as detected by flow cytometry. The results shown are from one experiment, representative of two independent experiments. *P<0.05; Student Two-Tailed T test, ns=not significant.

FIG. 23A-23D illustrate that Pip4k2c deficiency leads reduced tumor burden. FIG. 23A shows a western blot of Pip4k2c, Pip4k2a, and Pip4k2b proteins expressed in a melanoma line having a specific deletion of Pip4k2c by CRISPR-Cas9 using a Pip4k2c-specific guide RNA (sgPip4k2c cells). Control melanoma cells were treated with a scrambled guide RNA (sgScramble). As illustrated, the sgPip4k2c cells exhibited significantly reduced Pip4k2c expression compared to Pip4k2a and Pip4k2b protein expression levels or the sgScramble control. FIG. 23B graphically illustrates melanoma tumor sizes of the sgPip4k2c (Pip4k2c^{-/-}) cells compared to the sgScramble control as a function of time after implantation of the sgPip4k2c (Pip4k2^{-/-}) cells or control sgScramble cells into wild type mice. FIG. 23C graphically illustrates melanoma sgPip4k2c (Pip4k2c^{-/-}) tumor weight compared to the sgScramble control cells after implantation of the sgPip4k2c (Pip4k2c^{-/-}) or control cells into wild type mice. FIG. 23D graphically illustrates melanoma tumor sizes of the sgPip4k2c (Pip4k2c^{-/-}) cells compared to the sgScramble control as a function of time after implantation of the sgPip4k2c (Pip4k2c^{-/-}) or control cells into immunodeficient NSG mice.

FIG. 24A-24B illustrate that transfer of Pip4k2c deficient dendritic cells (DCs) leads to protective anti-tumor immunity. FIG. 24A is a schematic diagram illustrating implantation of B16OVA tumor cells into different wild mice at day 0 with randomization of the mice to receive PBS (control), OVA pulsed wild type dendritic cells (DC1) or Pip4k2c deficient dendritic cells (Pip4k2c^{-/-} DC1). FIG. 24B graphically illustrates B16OVA tumor sizes as a function of time since implantation of PBS (control), OVA pulsed wild type dendritic cells (Pip4k2c^{+/+} DC1) or Pip4k2c deficient dendritic cells (Pip4k2c^{-/-} DC1). Results are from one experiment. *P<0.05, **P<0.01 ***P<0.001 ANOVA).

### DETAILED DESCRIPTION

Compositions and methods are described herein that provide anti-tumor immunity. The compositions and methods involve inhibiting, knockdown, knockout, or degradation of the expression and/or function of Pip4k2c. Such compositions and methods are useful for treating and inhibiting the onset and progression of cancer.

The Pip4k2c is inhibited, knocked out or knocked down either in vitro or in vivo within myeloid cells. Myeloid cells such as monocytes, macrophages, neutrophils, and diverse sets of cells have been referred to as myeloid derived suppressor cells (MDSCs) because they are thought to drive local and systemic immunosuppression that can allow unchecked cancer cell growth. However, as shown herein inhibition or deletion of Pip4k2c in myeloid populations, specifically dendritic cells, and regulatory T cells led to profound tumor control in mice.

For example, a cell sample that includes myeloid cells, cancer cells, lymphocytes, regulatory T cells, dendritic cells, or progenitors thereof can be isolated from a subject, Pip4k2c can be knocked out in those cells to generate one or more modified Pip4kc^{-/-} cells, and the modified Pip4k2c^{-/-} cells can be returned to the subject. Such Pip4k2c-deficient cells would then resist the types of immunosuppression to which cells expressing Pip4k2c are vulnerable.

### Pip4k2c (Phosphatidylinositol 5-phosphate 4-kinase type-2 gamma)

The Pip4k2c enzyme is expressed in the endoplasmic reticulum within various cell types, including within adult mouse kidney, brain, testis, ovary, and heart, with lower levels in liver, spleen, thymus, colon, and lung cells. The Pip4k2c enzyme catalyzes the following reaction:
1,2-diacyl-sn-glycero-3-phospho-(1D-myo-inositol-5-phosphate)+ATP [symbol]
1,2-diacyl-sn-glycero-3-phospho-(1D-myo-inosito1-4,5-bisphosphate)+ADP+H⁺
   A sequence for a human Pip4k2c protein is available at the UniPROT website with accession number Q8TBX8-1 and is shown below as SEQ ID NO:1.

```
                10 20 30 40
MASSSVPPAT VSAATAGPGP GFGFASKTKK KHFVQQKVKV
                50 60 70 80
FRAADPLVGV FLWGVAHSIN ELSQVPPPVM LLPDDFKASS
                90 100 110 120
KIKVNNHLFH RENLPSHFKF KEYCPQVFRN LRDRFGIDDQ
              130 140 150 160
DYLVSLTRNP PSESEGSDGR FLISYDRTLV IKEVSSEDIA
              170 180 190 200
DMHSNLSNYH QYIVKCHGNT LLPQFLGMYR VSVDNEDSYM
              210 220 230 240
LVMRNMFSHR LPVHRKYDLK GSLVSREASD KEKVKELPTL
              250 260 270 280
KDMDFLNKNQ KVYIGEEEKK IFLEKLKRDV EFLVQLKIMD
              290 300 310 320
YSLLLGIHDI IRGSEPEEEA PVREDESEVD GDCSLIGPPA
              330 340 350 360
LVGSYGTSPE GIGGYIHSHR PLGPGEFESF IDVYAIRSAE
              370 380 390 400
GAPQKEVYFM GLIDILTQYD AKKKAAHAAK TVKHGAGAEI
              410 420
STVHPEQYAK RFLDFITNIF A
```

The Pip4k2c gene is located on human chromosome 12 (location 12q13.3; NC_000012.12 (57591188..57603418)). Genomic sequences encoding the human Pip4k2c protein are also available as accession numbers AC022506 and CH471054 in the NCBI database. A cDNA encoding the is available in the NCBI database as accession number AK297243.1, shown below as SEQ ID NO:2.

| | |
|---|---|
| 1 | GTCCGCTGTC CGGCCTCCGG TCACGTGACA GCAGCGCAGG |
| 41 | TGAGCGCCGC TTCCGGGGTC GGGCGCCTGG ATAGCTGCCG |
| 81 | GCTCCGGCTT CCACTTGGTC GGTTGCGCGG GAGACTATGG |
| 121 | CGTCCTCCTC GGTCCCACCA GCCACGGTAT CGGCGGCGAC |
| 161 | AGCAGGCCCC GGCCCAGGTT TCGGCTTCGC CTCCAAGACC |
| 201 | AAGAAGAAGC ATTTCGTGCA GCAGAAGGTG AAGGTGTTCC |
| 241 | GGGCGGCCGA CCCGCTGGTG GGTGTGTTCC TGTGGGGCGT |
| 281 | AGCCCACTCG ATCAATGAGC TCAGCCAGGT GCCTCCCCCG |
| 321 | GTGATGCTGC TGCCAGATGA CTTTAAGGCC AGCTCCAAGA |
| 361 | TCAAGGTCAA CAATCACCTT TTCCACAGGG AAAATCTGCC |
| 401 | CAGTCATTTC AAGTTCAAGG AGTATTGTCC CCAGGTCTTC |
| 441 | AGGAACCTCC GTGATCGATT TGGCATTGAT GACCAAGATT |
| 481 | ACTTGTACAT TGTGAAGTGC CATGGCAACA CGCTTCTGCC |
| 521 | CCAGTTCCTG GGGATGTACC GAGTCAGTGT GGACAACGAA |
| 561 | GACAGCTACA TGCTTGTGAT GCGCAATATG TTTAGCCACC |
| 601 | GTCTTCCTGT GCACAGGAAG TATGACCTCA AGGGTTCCCT |
| 641 | AGTGTCCCGG GAAGCCAGCG ATAAGGAAAA GGTTAAAGAA |
| 681 | TTGCCCACCC TTAAGGATAT GGACTTTCTC AACAAGAACC |
| 721 | AGAAAGTATA TATTGGTGAA GAGGAGAAGA AAATATTTCT |
| 761 | GGAGAAGCTG AAGAGAGATG TGGAGTTTCT AGTGCAGCTG |
| 801 | AAGATCATGG ACTACAGCCT TCTGCTAGGC ATCCACGACA |
| 841 | TCATTCGGGG CTCTGAACCA GAGGAGGAAG CGCCCGTGCG |
| 881 | GGAGGATGAG TCAGAGGIGG ATGGGGACTG CAGCCTGACT |
| 921 | GGACCTCCTG CTCTGGTGGG CTCCTATGGC ACCTCCCCAG |
| 961 | AGGGTATCGG AGGCTACATC CATTCCCATC GGCCCCTGGG |
| 1001 | CCCAGGAGAG TTTGAGTCCT TCATTGATGT CTATGCCATC |
| 1041 | CGGAGTGCTG AAGGAGCCCC CCAGAAGGAG GTCTACTTCA |
| 1081 | TGGGCCTCAT TGATATCCTT ACACAGTATG ATGCCAAGAA |
| 1121 | GAAAGCAGCT CATGCAGCCA AAACTGTCAA GCATGGGGCT |
| 1161 | GGGGCAGAGA TCTCTACTGT CCATCCGGAG CAGTATGCTA |
| 1201 | AGCGATTCCT GGATTTTATT ACCAACATCT TTGCCTAAGA |
| 1241 | GACTGCCTGG TTCTCTCTGA TGTTCAAGGT GGTGGGGTTC |
| 1281 | TGAGACACTT GGGGGAATTG TGGGGATATT CTAGCCACCA |
| 1321 | GTTCTCTTCT TCCTTTGCTA AATTCAGGCT GCAGGCTCCT |
| 1361 | TCCATCCAGA TAACTCCATC CTGTCGAGTA GGCTCTTTCT |
| 1401 | GACCCTCAGA AATACATTGT CCTTTTTCCT CTTTGCCCAT |
| 1441 | TTTTCTTCCC TCTCTTCCTC CCCATGAGAA GTCTGCTTGT |
| 1481 | AGTATTAGAA TGTTATTGTT GACTCTCTCC CAAGTGCCTT |
| 1521 | GATCTTTGTA ATATCTCCTG TIGTTTCTAT GATATAGG |

The Pip4k2c sequences can vary amongst the human population. Many such variants can include codon variations and/or conservative amino acid changes. However, the Pip4k2c sequences can also include non-conservative variations. For example, the Pip4k2c nucleic acids or Pip4k2c proteins can have at least 85% sequence identity and/or complementary, or at least 90% sequence identity and/or complementary, or at least 95% sequence identity and/or complementary, or at least 96% sequence identity and/or complementary, or at least 97% sequence identity and/or complementary, or at least 98% sequence identity and/or complementary, or at least 99% sequence identity and/or complementary to the target Pip4k2c nucleic acid or Pip4k2c protein.

### Binding Agents

Cell surface marker binding agents and Pip4k2c binding agents can be used to deliver Pip4k2c modifying agents to cells and/or to inhibit Pip4k2c function. For example, the binding agents can target Pip4k2c or myeloid cells, lymphocytes, regulatory T cells, dendritic cells, bone marrow cells, granulocytes, basophils, eosinophils, neutrophils, monocytes, mast cells, erythrocytes, macrophages, platelets, and combinations thereof. In some cases, the Pip4k2c binding agents can tag Pip4k2c for destruction, for example, by linking E3 ubiquitin ligase to Pip4k2c via the binding agent. In some cases, the cell binding agents can include a second agent that binds Pip4k2c so that Pip4k2c is targeted after the cell binding agent contacts the cell -thereby delivering to Pip4k2c a degrader that is part of the second agent.

Antibodies and polypeptides that bind specifically to myeloid cell surface markers or Pip4k2c can be used in the compositions and methods described herein. Such antibodies may be monoclonal antibodies. In some cases, the antibodies can be polyclonal antibodies. Such antibodies may also be humanized or fully human antibodies. The antibodies can exhibit one or more desirable functional properties, such as high affinity or specific binding to selected myeloid cell surface markers or to Pip4k2c.

Methods and compositions described herein can include anti-myeloid cell surface markers or anti-Pip4k2c antibodies, or a combination of such anti-myeloid cell surface markers or antibodies with agents that modify or degrade of Pip4k2c nucleic acids or proteins.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g. one or more myeloid cell surface markers or one or more Pip4k2c epitopes or domains). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds Pip4k2c or to one or more myeloid cell surface markers and is substantially free of antibodies that specifically bind antigens other than Pip4k2c or the myeloid cell surface markers). In some cases, the antibodies may however, have cross-reactivity to other antigens, such as Pip4k2c protein variants or Pip4k2c from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody or polypeptide that “specifically binds to a Pip4k2c” or “binds specifically to one or more myeloid cell surface markers” is intended to refer to an antibody or polypeptide that binds with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d}” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human Pip4k2c or bind specifically to one or more myeloid cell surface markers. Preferably, an antibody of the invention binds to Pip4k2c or binds specifically to one or more myeloid cell surface markers with high affinity, for example with a K_{D} of 1×10⁻⁷ M or less (e.g., less than 1×10⁻⁸ M or less than 1×10⁻⁹ M). The antibodies can exhibit one or more of the following characteristics:
(a) binds to one or more human Pip4k2c proteins with a K_{D} of 1×10⁻⁷ M or lower;
(b) binds to one or more human myeloid cell surface markers with a K_{D} of 1×10⁻⁷ M or lower;
(c) facilitates linkage of one or more types of Pip4k2c proteins to a degradation signal (e.g., E3 ubiquitin ligase);
(d) delivers one or more guide RNAs or anti-Pip4k2c antibodies to myeloid cells;
(e) enhances immune responses;
(f) reduces cancer cell growth or cancer progression; or
(g) a combination thereof.

Assays to evaluate the binding ability of the antibodies toward Pip4k2c or to myeloid cells can be used, including for example, ELISAs, Western blots and radioimmunoassays (RIAs). The binding kinetics (e.g., binding affinity) of the antibodies also can be assessed by standard assays known in the art, such as by Biacore™. analysis.

Given that the subject antibody preparations can bind specifically, the V_{L} and V_{H} sequences can be “mixed and matched” to create other binding molecules that bind with similar affinity. The binding properties of such “mixed and matched” antibodies can be tested using the binding assays (e.g., ELISAs). When V_{L} and V_{H} chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence; wherein the antibody specifically binds Pip4k2c or to a myeloid cell surface marker.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alpha v beta 3 antibodies using a heavy and light chain variable CDR3 domain. Hence, in some cases a mixed and matched antibody or a humanized antibody contains a CDR3 antigen binding domain that is specific for Pip4k2c or specific for a myeloid cell surface marker.

### Nucleic Acids that Inhibit Pip4k2c

Various inhibitors of Pip4k2c function can be employed in the compositions and methods described herein. For example, one type of Pip4k2c inhibitor can be an inhibitory nucleic acid. The expression or translation of an endogenous Pip4k2c can be inhibited, for example, by use of an inhibitory nucleic acid that specifically binds to an endogenous (target) nucleic acid that encodes Pip4k2c.

An inhibitory nucleic acid can have at least one segment that will hybridize to Pip4k2c nucleic acid under intracellular or stringent conditions. The inhibitory nucleic acid can reduce expression of a nucleic acid encoding Pip4k2c. An inhibitory nucleic acid may hybridize to a genomic DNA, a messenger RNA, or a combination thereof. An inhibitory nucleic acid may be incorporated into a plasmid vector or viral DNA. It may be single stranded or double stranded, circular, or linear.

An inhibitory nucleic acid is a polymer of ribose nucleotides or deoxyribose nucleotides having more than 13 nucleotides in length. An inhibitory nucleic acid may include naturally-occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a Pip4k2c nucleic acid. Such an inhibitory nucleic acid may be completely complementary to a segment of Pip4k2c nucleic acid (e.g., to a Pip4k2c mRNA). Alternatively, some variability is permitted in the inhibitory nucleic acid sequences relative to Pip4k2c sequences. For example, the Pip4k2c nucleic acids or Pip4k2c proteins can have at least 85% sequence identity and/or complementary, or at least 90% sequence identity and/or complementary, or at least 95% sequence identity and/or complementary, or at least 96% sequence identity and/or complementary, or at least 97% sequence identity and/or complementary, or at least 98% sequence identity and/or complementary, or at least 99% sequence identity and/or complementary to the target Pip4k2c nucleic acid.

An inhibitory nucleic acid can hybridize to a Pip4k2c nucleic acid under intracellular conditions or under stringent hybridization conditions and is sufficient to inhibit expression of a Pip4k2c nucleic acid. Intracellular conditions refer to conditions such as temperature, pH and salt concentrations typically found inside a cell, e.g. a target cell described herein.

Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a Pip4k2c coding or flanking sequence, can each be separated by a stretch of contiguous nucleotides that are not complementary to adjacent coding sequences, and such an inhibitory nucleic acid can still inhibit the function of a Pip4k2c nucleic acid. In general, each stretch of contiguous nucleotides is at least 4, 5, 6, 7, or 8 or more nucleotides in length. Non-complementary intervening sequences may be 1, 2, 3, or 4 nucleotides in length.

One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme, or an antisense nucleic acid molecule.

The inhibitory nucleic acid molecule may be single or double stranded (e.g. a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce Pip4k2c translation such that translation of the encoded polypeptide is reduced. SiRNAs mediate post-transcriptional gene silencing in a sequence-specific manner See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the Pip4k2c mRNA transcript. The region of homology may be 30 nucleotides or less in length, such as less than 25 nucleotides, or for example about 21 to 23 nucleotides in length. SiRNA is typically double stranded and may have two-nucleotide 3′ overhangs, for example, 3′ overhanging UU dinucleotides. Methods for designing siRNAs are available, see, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, California), can be used to make siRNA or shRNA for inhibiting Pip4k2c expression. The construction of the siRNA or shRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA or shRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA or shRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin or a shRNA. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, or about 3 to 23 nucleotides in length, and may include various nucleotide sequences including for example, AUG, CCC, UUCG, CCACC, CTCGAG, AAGCUU, and CCACACC. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally-occurring nucleotides, modified nucleotides, or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target Pip4k2c nucleic acid.

### Genomic Modification to Reduce Pip4k2c

In some cases, Pip4k2c expression of functioning can be reduced by genomic modification of one or more Pip4k2c genes.

Non-limiting examples of methods of introducing a modification into the genome of a cell can include use of microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety.

For example, nucleases such as zinc finger nucleases (ZFNs), transcription activator like effector nucleases (TALENs), and/or meganucleases can be employed with a guide nucleic acid that allows the nuclease to target the genomic Pip4k2c site(s). In some cases, a targeting vector can be used to introduce a deletion or modification of one or more genomic Pip4k2c site(s).

A “targeting vector” is a vector generally has a 5′ flanking region and a 3′ flanking region homologous to segments of the gene of interest. The 5′ flanking region and a 3′ flanking region can surround a DNA sequence comprising a modification and/or a foreign DNA sequence to be inserted into the gene. For example, the foreign DNA sequence may encode a selectable marker. In some cases, the targeting vector does not comprise a selectable marker, but such a selectable marker can facilitate identification and selection of cells with desirable mutations. Examples of suitable selectable markers include antibiotics resistance genes such as chloramphenicol resistance, gentamycin resistance, kanamycin resistance, spectinomycin resistance (SpecR), neomycin resistance gene (NEO), and/or the hygromycin β-phosphotransferase genes. The 5′ flanking region and the 3′ flanking region can be homologous to regions within the gene, or to regions flanking the gene to be deleted, modified, or replaced with the unrelated DNA sequence. The targeting vector is contacted with the native gene of interest in vivo (e.g., within the cell) under conditions that favor homologous recombination. For example, the cell can be contacted with the targeting vector under conditions that result in transformation of the cyanobacterial cell(s) with the targeting vector.

A typical targeting vector contains nucleic acid fragments of not less than about 0.1 kb nor more than about 10.0 kb from both the 5′ and the 3′ ends of the genomic locus which encodes the gene to be modified (e.g. the genomic Pip4k2c site(s)). These two fragments are separated by an intervening fragment of nucleic acid which encodes the modification to be introduced. When the resulting construct recombines homologously with the chromosome at this locus, it results in the introduction of the modification, e.g. a deletion of a portion of the genomic Pip4k2c site(s), replacement of the genomic Pip4k2c promoter or coding region site(s), or the insertion of non-conserved codon or a stop codon.

In some cases, a Cas9/CRISPR system can be used to create a modification in genomic Pip4k2c that reduces the expression or functioning of the Pip4k2c gene products. Clustered regularly interspaced short palindromic repeats (CRISPR)/CRISPR-associated (Cas) systems are useful for, e.g. RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is described, for example, by Mali et al. (Science 2013 339:823-6), which is incorporated by reference herein in its entirety. Kits for the design and use of CRISPR-mediated genome editing are commercially available, e.g. the PRECISION X CAS9 SMART NUCLEASE™ System (Cat No. CAS900A-1) from System Biosciences, Mountain View, CA.

In other cases, a cre-lox recombination system of bacteriophage P1, described by Abremski et al. 1983. Cell 32:1301 (1983), Sternberg et al., Cold Spring Harbor Symposia on Quantitative Biology, Vol. XLV 297 (1981) and others, can be used to promote recombination and alteration of the genomic Pip4k2c site(s). The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317 and 5,801,030), and in viral vectors (Hardy et al., J. Virology 71:1842 (1997).

The genomic mutations so incorporated can alter one or more amino acids in the encoded Pip4k2c gene products. For example, genomic sites modified so that in the encoded Pip4k2c protein is more prone to degradation, or is less stable, so that the half-life of such protein(s) is reduced. In another example, genomic sites can be modified so that at least one amino acid of a Pip4k2c polypeptide is deleted or mutated to reduce the enzymatic activity at least one type of Pip4k2c. In some cases, a conserved amino acid, or a conserved domain of the Pip4k2c polypeptide is modified. For example, a conserved amino acid or several amino acids in a conserved domain of the Pip4k2c polypeptide can be replaced with one or more amino acids having physical and/or chemical properties that are different from the conserved amino acid(s). For example, to change the physical and/or chemical properties of the conserved amino acid(s), the conserved amino acid(s) can be deleted or replaced by amino acid(s) of another class, where the classes are identified in the following Table 1.

TABLE 1

| Classification | Genetically Encoded |
|---|---|
| Hydrophobic | A, G, F, I, L, M, P, V, W |
| Aromatic | F, Y, W |
| Apolar | M, G, P |
| Aliphatic | A, V, L, I |
| Hydrophilic | C, D, E, H, K, N, Q, R, S, T, Y |
| Acidic | D, E |
| Basic | H, K, R |
| Polar | Q, N, S, T, Y |
| Cysteine-Like | C |

Different types of amino acids can be employed in the Pip4k2c polypeptide. Examples are shown in Table 2.

TABLE 2

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| N-Methylglycine | | MeGly |
| (sarcosine) | | |
| Ornithine | | Orn |
| Norleucine | | Nle |
| Penicillamine | | Pen |
| Homoarginine | | hArg |
| N-methylvaline | | MeVal |
| Homocysteine | | hCys |
| Homoserine | | hSer |

Such genomic modifications can reduce the expression or functioning of Pip4k2c gene products by at least 10%, or at least 15%, or at least 20%, or at least 25%, or at least 30%, or at least 35%, or at least 40%, or at least 45%, or at least 50% compared to the unmodified Pip4k2c gene product expression or functioning.

### Degradation of Pip4k2c

Although Pip4k2c genetic knockdown or knockout can be used to reduce the cellular concentration or amount of these proteins, in some cases post-translational disruption, degradation, or destabilization of Pip4k2c proteins can be preferable.

Targeting proteins directly, rather than via the DNA or mRNA molecules that encode them, can be a more direct and rapid method for reducing the scaffolding function of Pip4k2c proteins.

The Pip4k2c proteins can be directly disrupted, degraded, or destabilized in a variety of ways.

For example, Pip4k2c proteins can be degraded by tagging endogenous Pip4k2c proteins with an agent that signals cells to degrade the Pip4k2c proteins.

One example of an agent that signals cells to degrade the Pip4k2c proteins is an E3 ubiquitin ligase. Binding moieties can be used to link the degradation signal (e.g., E3 ubiquitin ligase) to the Pip4k2c proteins. Such binding moieties can be antibodies, peptides, polysaccharides, lipids, or small molecules that bind specifically Pip4k2c. Antibody-bound Pip4k2c proteins can be recognized, for example, by the cytosolic antibody receptor, TRIM21, which is an E3 ubiquitin ligase that binds with high affinity to the Fc domain of antibodies. Binding moieties can be linked to an E3 ubiquitin ligase to direct the E3 ubiquitin ligase to one or more Pip4k2c protein. Any binding moiety for Pip4k2c proteins can be adapted to directly or indirectly link or tag E3 ubiquitin ligase to the Pip4k2c proteins.

Small molecules that bind Pip4k2c proteins include those that are described, for example, in WO/2016/210291 and WO/2016/210296.

Methods for degradation or inhibition of Pip4k2c can include introducing a complex to a subject where the complex is a protein with E3 ubiquitin ligase activity that is linked to a binding moiety for Pip4k2c proteins to a subject or to a population of cells from a subject. Ubiquitination then occurs, and the Pip4k2c proteins are degraded.

Methods for degradation or inhibition of Pip4k2c can include inducing expression of an E3 ubiquitin ligase or introducing an exogenous an E3 ubiquitin ligase (e.g., TRIM21) expression system to a subject or into a population of cells from a subject and introducing an antibody for Pip4k2c proteins to a subject or to a population of cells from a subject. Ubiquitination then occurs followed by degradation of the antibody-bound Pip4k2c proteins.

For example, at least four E3 ligases (i.e., MDM2, IAP, VHL, and cereblon) can be used as tags for degradation of Pip4k2c proteins.

Mouse double minute 2 homolog (MDM2), also known as E3 ubiquitin-protein ligase Mdm2, is a nuclear-localized protein that in humans is encoded by the MDM2 gene. The encoded protein can promote tumor formation by targeting tumor suppressor proteins, such as p53, for proteasomal degradation. Mdm2 protein functions both as an E3 ubiquitin ligase that recognizes the N-terminal trans-activation domain (TAD) of the p53 tumor suppressor and an inhibitor of p53 transcriptional activation.

One example of sequence for a Homo sapiens E3 ubiquitin-protein ligase Mdm2 (isoform 2) is available as accession no. NP_001354919 XP_005268929 and shown below as SEQ ID NO:3.

| | |
|---|---|
| 1 | MCNTNMSVPT DGAVTTSQIP ASEQETLVRP KPLLLKLLKS |
| 41 | VGAQKDTYTM KEVLFYLGQY IMTKRLYDEK QQHIVYCSND |
| 81 | LLGDLFGVPS FSVKEHRKIY TMIYRNLVVV NQQESSDSGT |
| 121 | SVSENRCHLE GGSDQKDLVQ ELQEEKPSSS HLVSRPSTSS |
| 161 | RRRAISETEE NSDELSGERQ RKRHKSDSIS LSFDESLALC |
| 201 | VIREICCERS SSSESTGIPS NPDLDAGVSE HSGDWLDQDS |
| 241 | VSDQFSVEFE VESLDSEDYS LSEEGQELSD EDDEVYQVTV |
| 281 | YQAGESDTDS FEEDPEISLA DYWKCTSCNE MNPPLPSHCN |
| 321 | RCWALRENWL PEDKGKDKGE ISEKAKLENS TQAEEGFDVP |
| 361 | DCKKTIVNDS RESCVEENDD KITQASQSQE SEDYSQPSTS |
| 401 | SSIIYSSQED VKEFEREETQ DKEESVESSL PLNAIEPCVI |
| 441 | CQGRPKNGCI VHGKTGHLMA CFTCAKKLKK RNKPCPVCRQ |
| 481 | PIQMIVLTYF P |

Another example of a Homo sapiens E3 ubiquitin-protein ligase Mdm2 is available as accession no. CAP16727.1 and shown below as SEQ ID NO:4.

| | |
|---|---|
| 1 | MCNTNMSVPT DGAVTTSQIP ASEQETLVRP KPLLLKLLKS |
| 41 | VGAQKDTYTM KEFATKHRAK NIPV |

Another example of a Homo sapiens E3 ubiquitin-protein ligase Mdm2 is available as accession no. CAP16726.1 and shown below as SEQ ID NO:5.

| | |
|---|---|
| 1 | MCNTNMSVPT DGAVTTSQIP ASEQETLVRP KPLLLKLLKS |
| 41 | VGAQKDTYTM KENHRTQVHL |

Another example of a Homo sapiens E3 ubiquitin-protein ligase Mdm2 is available as accession no. CAP16725.1 and shown below as SEQ ID NO:6.

| | |
|---|---|
| 1 | MCNTNMSVPT DGAVTTSQIP ASEQETLVRP KPLLLKLLKS |
| 41 | VGAQKDTYTM KEENIYHDLQ ELGSSQSAGR KFR |

Inhibitors of Apoptosis Protein (IAPs) are guardian ubiquitin ligases that keep classic pro-apoptotic proteins in check, and regulate not only caspases and apoptosis, but also modulates inflammatory signaling and immunity, copper homeostasis, mitogenic kinase signaling, cell proliferation, as well as cell invasion and metastasis. IAPs can act as direct caspase inhibitors and can directly bind to the active site pocket of CASP3 and CASP7 to obstruct substrate entry. IAPs can also inactivate CASP9 by keeping it in a monomeric, inactive state. IAP acts as an E3 ubiquitin-protein ligase regulating NF-kappa-B signaling and the target proteins for its E3 ubiquitin-protein ligase activity include: RIPK1, CASP3, CASP7, CASP8, CASP9, MAP3K2/EKK2, DIABLO/SMAC, AIFM1, CCS and BIRC5/survivin. IAP plays a role in copper homeostasis by ubiquitinating COMMD1 and promoting its proteasomal degradation and can also function as E3 ubiquitin-protein ligase of the NEDD8 conjugation pathway, targeting effector caspases for neddylation and inactivation. IAP regulates the BMP signaling pathway and the SMAD and MAP3K7/TAK1 dependent pathways leading to NF-kappa-B and JNK activation.

One example of sequence for a Homo sapiens IAP E3 ubiquitin-protein ligase is available from the NCBI database as accession number P98170.2 and provided below as SEQ ID NO:7.

| | |
|---|---|
| 1 | MTFNSFEGSK TCVPADINKE EEFVEEFNRL KTFANFPSGS |
| 41 | PVSASTLARA GFLYTGEGDT VRCFSCHAAV DRWQYGDSAV |
| 81 | GRHRKVSPNC RFINGFYLEN SATQSTNSGI QNGQYKVENY |
| 121 | LGSRDHFALD RPSETHADYL LRTGQVVDIS DTIYPRNPAM |
| 161 | YSEEARLKSF QNWPDYAHLT PRELASAGLY YTGIGDQVQC |
| 201 | FCCGGKLKNW EPCDRAWSEH RRHFPNCFFV LGRNLNIRSE |
| 241 | SDAVSSDRNF PNSTNLPRNP SMADYEARIF TFGTWIYSVN |
| 281 | KEQLARAGFY ALGEGDKVKC FHCGGGLTDW KPSEDPWEQH |
| 321 | AKWYPGCKYL LEQKGQEYIN NIHLTHSLEE CLVRTTEKTP |
| 361 | SLTRRIDDTI FQNPMVQEAI RMGFSFKDIK KIMEEKIQIS |
| 401 | GSNYKSLEVL VADLVNAQKD SMQDESSQTS LQKEISTEEQ |
| 441 | LRRLQEEKLC KICMDRNIAI VFVPCGHLVT CKQCAEAVDK |
| 481 | CPMCYTVITF KQKIFMS |

Another example of a Homo sapiens IAP E3 ubiquitin-protein ligase is available as accession no. Q13490.2 and shown below as SEQ ID NO:8.

| | |
|---|---|
| 1 | MHKTASQRLF PGPSYQNIKS IMEDSTILSD WINSNKQKMK |
| 41 | YDFSCELYRM STYSTFPAGV PVSERSLARA GFYYTGVNDK |
| 81 | VKCFCCGLML DNWKLGDSPI QKHKQLYPSC SFIQNLVSAS |
| 121 | LGSTSKNTSP MRNSFAHSLS PTLEHSSLFS GSYSSLSPNP |
| 161 | LNSRAVEDIS SSRINPYSYA MSTEEARFLT YHMWPLTFLS |
| 201 | PSELARAGFY YIGPGDRVAC FACGGKLSNW EPKDDAMSEH |
| 241 | RRHFPNCPFL ENSLETLRFS ISNLSMQTHA ARMRTFMYWP |
| 281 | SSVPVQPEQL ASAGFYYVGR NDDVKCFCCD GGLRCWESGD |
| 321 | DPWVEHAKWF PRCEFLIRMK GQEFVDEIQG RYPHLLEQLL |
| 361 | STSDTTGEEN ADPPIIHFGP GESSSEDAVM MNTPVVKSAL |
| 401 | EMGFNRDLVK QTVQSKILTT GENYKTVNDI VSALLNAEDE |
| 441 | KREEEKEKQA EEMASDDLSL IRKNRMALFQ QLTCVLPILD |
| 481 | NLLKANVINK QEHDIIKQKT QIPLQARELI DTILVKGNAA |
| 521 | ANIFKNCLKE IDSTLYKNLF VDKNMKYIPT EDVSGLSLEE |
| 561 | QLRRLQEERT CKVCMDKEVS VVFIPCGHLV VCQECAPSLR |
| 601 | KCPICRGIIK GTVRTFLS |

Another example of a Homo sapiens IAP E3 ubiquitin-protein ligase is available as accession no. Q96CA5.2 and shown below as SEQ ID NO:9.

| | |
|---|---|
| 1 | MGPKDSAKCL HRGPQPSHWA AGDGPTQERC GPRSLGSPVL |
| 41 | GLDTCRAWDH VDGQILGQLR PLTEEEEEEG AGATLSRGPA |
| 81 | FPGMGSEELR LASFYDWPLT AEVPPELLAA AGFFHIGHQD |
| 121 | KVRCFFCYGG LQSWKRGDDP WTEHAKWFPS CQFLLRSKGR |
| 161 | DFVHSVQETH SQLLGSWDPW EEPEDAAPVA PSVPASGYPE |
| 201 | LPTPRREVQS ESAQEPGGVS PAEAQRAWWV LEPPGARDVE |
| 241 | AQLRRLQEER TCKVCLDRAV SIVFVPCGHL VCAECAPGLQ |
| 281 | LCPICRAPVR SRVRTFLS |

The von Hippel-Lindau (VHL) tumor suppressor includes the substrate recognition subunit/E3 ligase complex VCB, which includes elongins B and C, and a complex including Cullin-2 and Rbx1. The primary substrate of VHL is Hypoxia Inducible Factor 1a (HIF-1a), a transcription factor that upregulates genes such as the pro-angiogenic growth factor VEGF and the red blood cell inducing cytokine erythropoietin in response to low oxygen levels.

One example of sequence for a Homo sapiens VHL E3 ubiquitin-protein ligase is available from the NCBI database as accession number NP_000542.1 and provided below as SEQ ID NO:10.

| | |
|---|---|
| 1 | MPRRAENWDE AEVGAEEAGV EEYGPEEDGG EESGAEESGP |
| 41 | EESGPEELGA EEEMEAGRPR PVLRSVNSRE PSQVIFCNRS |
| 81 | PRVVLPVWLN FDGEPQPYPT LPPGTGRRIH SYRGHLWLFR |
| 121 | DAGTHDGLLV NQTELFVPSL NVDGQPIFAN ITLPVYTLKE |
| 161 | RCLQVVRSLV KPENYRRLDI VRSLYEDLED HPNVQKDLER |
| 201 | LTQERIAHQR MGD |

Another example of a Homo sapiens VHL E3 ubiquitin-protein ligase is available as accession no. NP_937799.1 and shown below as SEQ ID NO:11.

| | |
|---|---|
| 1 | MPRRAENWDE AEVGAEEAGV EEYGPEEDGG EESGAEESGP |
| 41 | EESGPEELGA EEEMEAGRPR PVLRSVNSRE PSQVIFCNRS |
| 81 | PRVVLPVWLN FDGEPQPYPT LPPGTGRRIH SYRVYTLKER |
| 121 | CLQVVRSLVK PENYRRLDIV RSLYEDLEDH PNVQKDLERL |
| 161 | TQERIAHQRM GD |

Another example of a Homo sapiens VHL E3 ubiquitin-protein ligase is available as accession no. NP_001341652.1 and shown below as SEQ ID NO:12.

| | |
|---|---|
| 1 | MPRRAENWDE AEVGAEEAGV EEYGPEEDGG EESGAEESGP |
| 41 | EESGPEELGA EEEMEAGRPR PVLRSVNSRE PSQVIFCNRS |
| 81 | PRVVLPVWLN FDGEPQPYPT LPPGTGRRIH SYRVLMTPVG |
| 121 | QFCVVPALVE NTFLLGRLTD AKTGTSQGHV GAGRADRVWR |
| 161 | GKLTYLPAGR WRGCGCVVSV KEHFPEKEES RME |

Cereblon is a protein that in humans is encoded by the CRBN gene. Cereblon proteins are related to the Lon protease protein family In mammals cereblon is found in the cytoplasm localized with a calcium channel membrane protein and is thought to play a role in brain development. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

One example of sequence for a Homo sapiens cereblon E3 ubiquitin-protein ligase is available from the NCBI database as accession number NP_057386.2 and provided below as SEQ ID NO:13.

| | |
|---|---|
| 1 | MAGEGDQQDA AHNMGNHLPL LPAESEEEDE MEVEDQDSKE |
| 41 | AKKPNIINFD TSLPTSHTYL GADMEEFHGR TLHDDDSCQV |
| 81 | IPVLPQVMMI LIPGQTLPLQ LFHPQEVSMV RNLIQKDRTF |
| 121 | AVLAYSNVQE REAQFGTTAE IYAYREEQDF GIEIVKVKAI |
| 161 | GRQRFKVLEL RTQSDGIQQA KVQILPECVL PSTMSAVQLE |
| 201 | SLNKCQIFPS KPVSREDQCS YKWWQKYQKR KFHCANLTSW |
| 241 | PRWLYSLYDA ETLMDRIKKQ LREWDENLKD DSLPSNPIDE |
| 281 | SYRVAACLPI DDVLRIQLLK IGSAIQRLRC ELDIMNKCTS |
| 321 | LCCKQCQETE ITTKNEIFSL SLCGPMAAYV NPHGYVHETL |
| 361 | TVYKACNLNL IGRPSTEHSW FPGYAWTVAQ CKICASHIGW |
| 401 | KFTATKKDMS PQKFWGLTRS ALLPTIPDTE DEISPDKVIL |
| 441 | CL |

Another example of a Homo sapiens cereblon E3 ubiquitin-protein ligase is available as accession no. NP_001166953.1 and shown below as SEQ ID NO:14.

| | |
|---|---|
| 1 | MAGEGDQQDA AHNMGNHLPL LPESEEEDEM EVEDQDSKEA |
| 41 | KKPNIINFDT SLPTSHTYLG ADMEEFHGRT LHDDDSCQVI |
| 81 | PVLPQVMMIL IPGQTLPLQL FHPQEVSMVR NLIQKDRTFA |
| 121 | VLAYSNVQER EAQFGTTAEI YAYREEQDFG IEIVKVKAIG |
| 161 | RQRFKVLELR TQSDGIQQAK VQILPECVLP STMSAVQLES |
| 201 | LNKCQIFPSK PVSREDQCSY KWWQKYQKRK FHCANLTSWP |
| 241 | RWLYSLYDAE TLMDRIKKQL REWDENLKDD SLPSNPIDFS |
| 281 | YRVAACLPID DVLRIQLLKI GSAIQRLRCE LDIMNKCTSL |
| 321 | CCKQCQETEI TTKNEIFSLS LCGPMAAYVN PHGYVHETLT |
| 361 | VYKACNLNLI GRPSTEHSWF PGYAWTVAQC KICASHIGWK |
| 401 | FTATKKDMSP QKFWGLTRSA LLPTIPDTED EISPDKVILC |
| 441 | L |

Another example of a Homo sapiens cereblon E3 ubiquitin-protein ligase is available as accession no. XP_005265259.1 and shown below as SEQ ID NO:15.

| | |
|---|---|
| 1 | MEEFHGRTLH DDDSCQVIPV LPQVMMILIP GQTLPLQLFH |
| 41 | PQEVSMVRNL IQKDRTFAVL AYSNVQEREA QFGTTAEIYA |
| 81 | YREEQDFGIE IVKVKAIGRQ RFKVLELRTQ SDGIQQAKVQ |
| 121 | ILPECVLPST MSAVQLESLN KCQIFPSKPV SREDQCSYKW |
| 161 | WQKYQKRKFH CANLISWPRW LYSLYDAETL MDRIKKQLRE |
| 201 | WDENLKDDSL PSNPIDFSYR VAACLPIDDV LRIQLLKIGS |
| 241 | AIQRLRCELD IMNKCTSLCC KQCQETEITT KNEIFSLSLC |
| 281 | GPMAAYVNPH GYVHETLTVY KACNLNLIGR PSTEHSWFPG |
| 321 | YAWTVAQCKI CASHIGWKFT ATKKDMSPQK FWGLTRSALL |
| 361 | PTIPDTEDEI SPDKVILCL |

Another example of a Homo sapiens cereblon E3 ubiquitin-protein ligase is available as accession no. XP_011532093.1 and shown below as SEQ ID NO:16.

| | |
|---|---|
| 1 | MAGEGDQQDA AHNMGNHLPL LPAESEEEDE MEVEDQDSKE |
| 41 | AKKPNIINFD TSLPTSHTYL GADMEEFHGR TLHDDDSCQV |
| 81 | IPVLPQVMMI LIPGQTLPLQ LFHPQEVSMV RNLIQKDRTF |
| 121 | AVLAYSNVQE REAQFGTTAE IYAYREEQDF GIEIVKVKAI |
| 161 | GRQRFKVLEL RTQSDGIQQA KVQILPECVL PSTMSAVQLE |
| 201 | SLNKCQIFPS KPVSREDQCS YKWWQKYQKR KFHCANLTSW |
| 241 | PRWLYSLYDA ETLMDRIKKQ LREWDENLKD DSLPSNPIDE |
| 281 | SYRVAACLPI DDVLRIQLLK IGSAIQRLRC ELDIMNKCTS |
| 321 | LCCKQCQETE ITTKNEIFRY AWTVAQCKIC ASHIGWKFTA |
| 361 | TKKDMSPQKF WGLTRSALLP TIPDTEDEIS PDKVILCL |

As described above, antibody-bound Pip4k2c proteins can be recognized by the cytosolic antibody receptor, TRIM21, which is an E3 ubiquitin ligase that binds with high affinity to the Fc domain of antibodies. Treatment with an antibody that binds specification to a Pip4k2c protein, either with or before administering or inducing the expression of TRIM21 can lead to degradation of the Pip4k2c protein.

One example of sequence for a Homo sapiens E3 ubiquitin-protein ligase TRIM21 polypeptide sequence is available from the NCBI database as accession number NP_003132.2 and provided below as SEQ ID NO:17.

| | |
|---|---|
| 1 | MASAARLIMM WEEVTCPICL DPFVEPVSIE CGHSFCQECI |
| 41 | SQVGKGGGSV CPVCRQRFLL KNLRPNRQLA NMVNNLKEIS |
| 81 | QEAREGTQGE RCAVHGERLH LFCEKDGKAL CWVCAQSRKH |
| 121 | RDHAMVPLEE AAQEYQEKLQ VALGELRRKQ ELAEKLEVEI |
| 161 | AIKRADWKKT VETQKSRIHA EFVQQKNFLV EEEQRQLQEL |
| 201 | EKDEREQLRI LGEKEAKLAQ QSQALQELIS ELDRRCHSSA |
| 241 | LELLQEVIIV LERSESWNLK DLDITSPELR SVCHVPGLKK |
| 281 | MLRTCAVHIT LDPDTANPWL ILSEDRRQVR LGDTQQSIPG |
| 321 | NEERFDSYPM VLGAQHFHSG KHYWEVDVTG KEAWDLGVCR |
| 361 | DSVRRKGHFL LSSKSGFWTI WLWNKQKYEA GTYPQTPLHL |
| 401 | QVPPCQVGIF LDYEAGMVSF YNITDHGSLI YSFSECAFTG |
| 441 | PLRPFFSPGF NDGGKNTAPL TLCPLNIGSQ GSTDY |

Similarly, a PROteolysis-TArgeting Chimeras (PROTACs) system can be used to tag one or more of the Pip4k2c for selective degradation. The PROTAC systems include a ligand to the target Pip4k2c protein, a ligand to the E3 ubiquitin ligase, and a linker connecting the two ligands. See, e.g., Bondeson & Crew, Annu Rev Pharmacol Toxicol. 57: 107-123 (2017).

Fragments of E3 ubiquitin ligases that can induce ubiquitination can also be used. For example, the E3 ubiquitin ligases include those that have at least 20, at least 22, at least 25, at least 27, at least 30, at least 35, at least 40, at least 50 of the same amino acids as an E3 ubiquitin ligase. The identical amino acids can be distributed throughout the E3 ubiquitin ligases and need not be contiguous but are present in homologous positions.

The at least 70%, 80%, 90%, 95%, 96%, 97%, 98%, 99% or more than 99% sequence identity to any of the E3 ubiquitin ligases described herein, or at least 70%, 80%, 90%, 95%, 96%, 97%, 98%, 99% or more than 99% sequence identity to a fragment of an E3 ubiquitin ligase that has at least 20, at least 22, at least 25, at least 27, at least 30, at least 35, at least 40, at least 50 amino acids.

Expression vectors that include a nucleic acid segment that encodes any of these E3 ubiquitin ligase proteins can in some cases be used to increase expression of the E3 ubiquitin ligase proteins.

Thus, Pip4k2c degraders can be used to knockdown or knockout Pip4k2c. include a protein targeting ligand linked to an E3 ligase recruiter, where the targeting ligand brings the E3 ligase to Pip4k2c or cells expressing Pip4k2c to ubiquitinate and degrade the Pip4k2c or the Pip4k2c-expressing cells. in a proteasome-dependent manner For example, the E3 ubiquitin-protein ligase can be the E3 ubiquitin-protein ligase RNF114.

### Cellular Targets

Cellular targets for modulation, Pip4k2c modifying agents, and/or therapeutic agents can be myeloid cells. Myeloid cells include, for example, dendritic cells, bone marrow cells, granulocytes, basophils, eosinophils, neutrophils, monocytes, mast cells, erythrocytes, macrophages, platelets, and combinations thereof. In some cases, macrophages, dendritic cell, or T cells are targeted by the Pip4k2c modifying agents.

In some cases, myeloid cell markers can be linked to Pip4k2c-modifying agent to target those agents to the myeloid cells. Examples of myeloid cell markers include the mannose receptor (CD206), aminopeptidase N/CD13, CCR2, CCR3, CD11b/Integrin alpha M, CD14, CD34, CD36/SR-B3, CD38, CD44, CD59, CD68/SR-D1, CD69, CD117/c-kit, CD163, CD164, CD42b/GPIb alpha, CEACAM-1/CD66a, CEACAM-3/CD66d, CEACAM-5/CD66e, CEACAM-6/CD66c, CEACAM-8/CD66b, CXCR3, EMR1, F4/80, Fc gamma RIII (CD16), Fc gamma RIIIA/CD16a, Fc gamma RIIIB/CD16b, Flt-3/Flk-2, Glycophorin A, Glycoprotein V/CD42d, GP1BB, IL-3R alpha, Integrin alpha 2b/CD41, Integrin beta 2/CD18, Integrin beta 3/CD61, LAMP-1/CD107a, Ly-6G (Gr-1), Ly-6G/Ly-6C (Gr-1), myeloperoxidase/MPO, PEAR1, PS G1, PSG2, PSG3, PSGS, L-Selectin/CD62L, Siglec-3/CD33, thrombopoietin/Tpo, or a combination thereof. Antibodies that can bind these myeloid cell markers are available, for example, from R&D Systems (see rndsystems.com/research-area/myeloid-lineage-markers website).

Dendritic cells (DCs) are antigen-presenting cells (also called accessory cells) that process and present antigens on their cell surfaces to the T cells. Only the dendritic cells have the capacity to induce a primary immune response in inactive or resting naïve T lymphocytes. Dendritic cells therefore act as messengers between the innate and the adaptive immune systems.

Examples of dendritic cell markers include blood dendritic cell antigen 2 (BDCA-2), CD8, CD8-alpha, CD11b, CD11c, CD103, CD205, MHC Class II molecules, or a combination thereof.

Such myeloid markers and/or dendritic cell markers can be targets for delivery of agents that can modify Pip4k2c expression or function.

The myeloid cells can also be adapted to facilitate modification of Pip4k2c expression or function. For example, the myeloid cells can be modified to express one or more cas nucleases, for example, before or during introduction of one or more guide RNAs, or an expression therefor. In another example, the myeloid cells are modified to express one or more E3 ubiquitin ligase proteins.

### Pip4k2c Modifying Agents

Pip4k2c modifying agents reduce the expression or functioning of Pip4k2c. For example, the Pip4k2c modifying agents can knockout or knockdown the expression of Pip4k2c. The Pip4k2c modifying agents can include anti-Pip4k2c antibodies, Pip4k2c nucleic acid inhibitors, Pip4k2c degraders, Pip4k2c vaccines, small molecule Pip4k2c inhibitors, Pip4k2c guide RNAs with cas nucleases, and combinations thereof.

Such Pip4k2c modifying agents reduce the expression or functioning of Pip4k2c by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or by 100%.

### Methods for Treatment of Cancer

Modification of Pip4k2c genomic sites, inhibition of Pip4k2c nucleic acids, or degradation or depletion of Pip4k2c proteins is useful for preventing, treating and/or diagnosing cancer. Degradation or depletion of the Pip4k2c protein can enhance immune responses against cancer and tumors. Thus, one aspect of the invention is a method of treating or inhibiting the establishment and/or growth metastatic tumors in an animal (e.g., a human) Such a method involves administering compositions to the animal that modify, inhibit, degrade, or deplete Pip4k2c nucleic acids or proteins to thereby treat or inhibit the establishment and/or growth of cancer in an animal. Both human and veterinary uses are contemplated.

Methods are described herein for the treatment of cancer and to inhibit the progression of cancer. The methods of treating or inhibiting the progression of cancer and/or the establishment of metastatic tumors in an animal can include administering to a subject animal (e.g., a human), a therapeutically effective amount of a composition that degrades or depletes Pip4k2c protein. The methods of treating or inhibiting the establishment and/or growth metastatic tumors in an animal can also include administering such a composition with one or more other anti-cancer or chemotherapeutic agents.

In some embodiments, the methods can also include a detection step to ascertain whether the animal has cancer or is in need of treatment to inhibit the development of metastatic tumors. Such a detection step can include any available assay for cancer.

The term “animal” as used herein, refers to an animal, such as a warm-blooded animal, which is has a disease or condition, for example, cancer. Mammals include cattle, buffalo, sheep, goats, pigs, horses, dogs, cats, rats, rabbits, mice, and humans. Also included are other livestock, domesticated animals, and captive animals. The term “farm animals” includes chickens, turkeys, fish, and other farmed animals. Mammals and other animals including birds may be treated by the methods and compositions described and claimed herein. In some embodiments, the animal is a human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. In addition, a metastatic cancer at any stage of progression can be treated, such as micrometastatic tumors, megametastatic tumors, and recurrent cancers.

However, in some cases the degradation, modification, or inhibition of Pip4k2c is better targeted to myeloid cells. Such myeloid cells include dendritic cells, bone marrow cells, granulocytes, basophils, eosinophils, neutrophils, monocytes, mast cells, erythrocytes, macrophages, platelets, and combinations thereof.

Such modification, inhibition, degradation can improve immune function to treat a variety of cancer types. For example, the inventive methods and compositions can be used to treat cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, hematological malignancies, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. Information regarding numerous types of cancer can be found, e.g., from the American Cancer Society (www.cancer.org), or from, e.g., Wilson et al. (1991) Harrison's Principles of Internal Medicine, 12th Edition, McGraw-Hill, Inc.

The term “hematological malignancies” includes childhood leukemia and lymphomas, myeloid leukemia, Hodgkin's disease, lymphomas of lymphocytic and cutaneous origin, acute and chronic leukemia, plasma cell neoplasm and cancers associated with AIDS.

Treatment of, or treating, cancer can include the reduction in cancer cell growth, cancer cell migration, or the reduction in establishment of at least one metastatic tumor. The treatment also includes alleviation or diminishment of more than one symptom of cancer such as coughing, shortness of breath, hemoptysis, lymphadenopathy, enlarged liver, nausea, jaundice, bone pain, bone fractures, headaches, seizures, systemic pain, and combinations thereof. The treatment may cure the cancer, e.g., it may prevent cancer, it may substantially eliminate tumor formation and growth, and/or it may arrest or inhibit the migration of metastatic cancer cells.

Anti-cancer activity can be evaluated against a variety of cancers using methods available to one of skill in the art. Anti-cancer activity, for example, can determined by identifying the lethal dose (LD100) or the 50% effective dose (ED50) or the dose that inhibits growth at 50% (GI50) of a composition or agent of the present invention. In one aspect, anti-cancer activity is the amount of the agent that reduces 50%, 60%, 70%, 80%, 90%, 95%, 97%, 98%, 99% or 100% of cancer cell growth or migration, for example, when measured by detecting the level of expression of a cancer cell marker or the expression of a cancer cell marker at sites distal from a primary tumor site, or when assessed using available methods for detecting metastases.

In some cases, the compositions and methods described herein can reduce the symptoms of cancer and/or the tumor loads by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or by 100%.

The compositions described herein for treatment of cancer can include additional therapeutic agents such as additional anti-cancer or chemotherapeutic agents, vitamins, pain reducing agents, and anti-microbial agents.

The anti-cancer agents useful in the compositions and methods described herein include cytotoxins, photosensitizing agents and chemotherapeutic agents. These agents include, but are not limited to, folate antagonists, pyrimidine antimetabolites, purine antimetabolites, 5-aminolevulinic acid, alkylating agents, platinum anti-tumor agents, anthracyclines, DNA intercalators, epipodophyllotoxins, DNA topoisomerases, microtubule-targeting agents, vinca alkaloids, taxanes, epothilones and asparaginases. Further information can be found in Bast et al., Cancer Medicine, edition 5, which is available free as a digital book (see website at ncbi.nlm nih.gov/books/NBK20812/).

Folic acid antagonists are cytotoxic drugs used as antineoplastic, antimicrobial, anti-inflammatory, and immune-suppressive agents. While several folate antagonists have been developed, and several are now in clinical trial, methotrexate (MTX) is the antifolate with the most extensive history and widest spectrum of use. MTX is an essential drug in the chemotherapy regimens used to treat patients with acute lymphoblastic leukemia, lymphoma, osteosarcoma, breast cancer, choriocarcinoma, and head and neck cancer, as well as being an important agent in the therapy of patients with nonmalignant diseases, such as rheumatoid arthritis, psoriasis, and graft-versus-host disease.

Pyrimidine antimetabolites include fluorouracil, cytosine arabinoside, 5-azacytidine, and 2′,2′-difluoro-2′-deoxycytidine. Purine antimetabolites include 6-mercatopurine, thioguanine, allopurinol (4-hydroxypyrazolo-3,4-d-pyrimidine), deoxycoformycin (pentostatin), 2-fluoroadenosine arabinoside (fludarabine; 9-β-d-arabinofuranosyl-2-fluoradenine), and 2-chlorodeoxyadenosine (Cl-dAdo, cladribine). In addition to purine and pyrimidine analogues, other agents have been developed that inhibit biosynthetic reactions leading to the ultimate nucleic acid precursors. These include phosphonacetyl-L-aspartic acid (PALA), brequinar, acivicin, and hydroxyurea.

Alkylating agents and the platinum anti-tumor compounds form strong chemical bonds with electron-rich atoms (nucleophiles), such as sulfur in proteins and nitrogen in DNA. Although these compounds react with many biologic molecules, the primary cytotoxic actions of both classes of agents appear to be the inhibition of DNA replication and cell division produced by their reactions with DNA. However, the chemical differences between these two classes of agents produce significant differences in their anti-tumor and toxic effects. The most frequently used alkylating agents are the nitrogen mustards. Although thousands of nitrogen mustards have been synthesized and tested, only five are commonly used in cancer therapy today. These are mechlorethamine (the original “nitrogen mustard”), cyclophosphamide, ifosfamide, melphalan, and chlorambucil. Closely related to the nitrogen mustards are the aziridines, which are represented in current therapy by thiotepa, mitomycin C, and diaziquone (AZQ). Thiotepa (triethylene thiophosphoramide) has been used in the treatment of carcinomas of the ovary and breast and for the intrathecal therapy of meningeal carcinomatosis. The alkyl alkane sulfonate, busulfan, was one of the earliest alkylating agents. This compound is one of the few currently used agents that clearly alkylate through an SN2 reaction. Hepsulfam, an alkyl sulfamate analogue of busulfan with a wider range of anti-tumor activity in preclinical studies, has been evaluated in clinical trials but thus far has demonstrated no superiority to busulfan.

Photosensitizing agents induce cytotoxic effects on cells and tissues. Upon exposure to light the photosensitizing compound may become toxic or may release toxic substances such as singlet oxygen or other oxidizing radicals that are damaging to cellular material or biomolecules, including the membranes of cells and cell structures, and such cellular or membrane damage can eventually kill the cells. A range of photosensitizing agents can be used, including psoralens, porphyrins, chlorines, aluminum phthalocyanine with 2 to 4 sulfonate groups on phenyl rings (e.g., AlPcS2a or AlPcS4), and phthalocyanins. Such drugs become toxic when exposed to light. For example, the photosensitizing agent can be an amino acid called 5-aminolevulinic acid, which is converted to protoporphyrin IX, a fluorescent photosensitizer. The structure of 5-aminolevulinic acid is shown below.

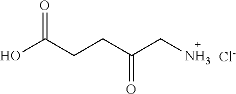

Topoisomerase poisons are believed to bind to DNA, the topoisomerase, or either molecule. Many topoisomerase poisons, such as the anthracyclines and actinomycin D, are relatively planar hydrophobic compounds that bind to DNA with high affinity by intercalation, which involves stacking of the compound between adjacent base pairs. Anthracyclines intercalate into double-stranded DNA and produce structural changes that interfere with DNA and RNA syntheses. Several of the clinically relevant anthracyclines are shown below.

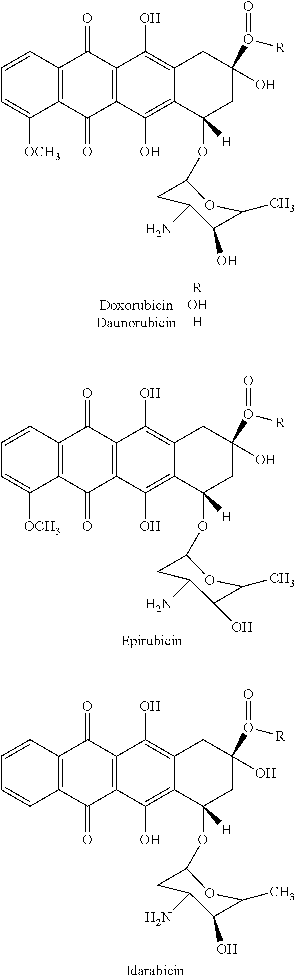

Non-intercalating topoisomerase-targeting drugs include epipodophyllotoxins such as etoposide and teniposide. Etoposide is approved in the United States for the treatment of testicular and small cell lung carcinomas. Etoposide phosphate is more water soluble than etoposide and is rapidly converted to etoposide in vivo. Other non-intercalating topoisomerase-targeting drugs include topotecan and irinotecan.

Unique classes of natural product anticancer drugs have been derived from plants. As distinct from those agents derived from bacterial and fungal sources, the plant products, represented by the Vinca and Colchicum alkaloids, as well as other plant-derived products such as paclitaxel (Taxol) and podophyllotoxin, do not target DNA. Rather, they either interact with intact microtubules, integral components of the cytoskeleton of the cell, or with their subunit molecules, the tubulins. Clinically useful plant products that target microtubules include the Vinca alkaloids, primarily vinblastine (VLB), vincristine (VCR), vinorelbine (Navelbine, VRLB), and a newer Vinca alkaloid, vinflunine (VFL; 20′,20′-difluoro-3′,4′-dihydrovinorelbine), as well as the two taxanes, paclitaxel and docetaxel (Taxotere). The structure of paclitaxel is provided below.

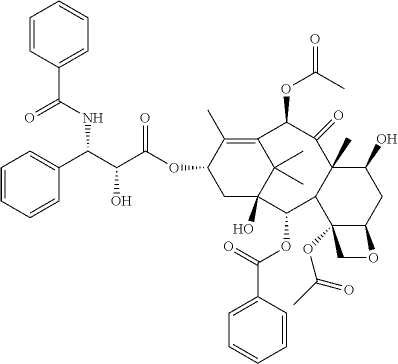 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Preferably a paclitaxel moiety is linked to the peptide by C10 and/or C2 hydroxyl moiety.

Examples of drugs that can be used in the methods and compositions described herein include but are not limited to, aldesleukin, 5-aminolevulinic acid, asparaginase, bleomycin sulfate, camptothecin, carboplatin, carmustine, cisplatin, cladribine, cyclophosphamide (lyophilized), cyclophosphamide (non-lyophilized), cytarabine (lyophilized powder), dacarbazine, dactinomycin, daunorubicin, diethylstilbestrol, doxorubicin (doxorubicin, 4′-epidoxorubicin, 4- or 4′-deoxydoxorubicin), epoetin alfa, esperamycin, etidronate, etoposide, N,N-bis(2-chloroethyl)-hydroxyaniline, 4-hydroxycyclophosphamide, fenoterol, filgrastim, floxuridine, fludarabine phosphate, fluorocytidine, fluorouracil, fluorouridine, goserelin, granisetron hydrochloride, idarubicin, ifosfamide, interferon alpha-2a, interferon alpha-2b, leucovorin calcium, leuprolide, levamisole, mechiorethamine, medroxyprogesterone, melphalan, methotrexate, mitomycin, mitoxantrone, muscarine, octreotide, ondansetron hydrochloride, oxyphenbutazone, paclitaxel, pamidronate, pegaspargase, plicamycin, salicylic acid, salbutamol, sargramostim, streptozocin, taxol, terbutaline, terfenadine, thiotepa, teniposide, vinblastine, vindesine and vincristine. Other drugs that can be used in the methods and compositions described herein include those, for example, disclosed in WO 98/13059; Payne, 2003; US 2002/0147138 and other references available to one of skill in the art.

### Compositions

The Pip4k2c degrading agents, inhibitors, mutating agents (e.g., guide RNAs), and/or binding (e.g., antibody) agents can be formulated as compositions with or without additional therapeutic agents, and administered to an animal, such as a human patient, in a variety of forms adapted to the chosen route of administration. Routes for administration include, for example, oral, local, parenteral, intraperitoneal, intravenous and intraarterial routes.

The compositions can be formulated as pharmaceutical dosage forms. Such pharmaceutical dosage forms can include (a) liquid solutions; (b) tablets, sachets, or capsules containing liquids, solids, granules, or gelatin; (c) suspensions in an appropriate liquid; and (d) suitable emulsions.

Solutions of the active agents (e.g., Pip4k2c degrading agents, Pip4k2c antibodies, Pip4k2c inhibitors, Pip4k2c guide RNAs, and other therapeutic agents) can be prepared in water or saline, and optionally mixed with other agents. For example, formulations for intravenous or intraarterial administration may include sterile aqueous solutions that may also contain buffers, diluents, stabilizing agents, nontoxic surfactants, chelating agents, polymers and/or other suitable additives. Sterile injectable solutions are prepared by incorporating the active agents in the required amount in the appropriate solvent with various of the other ingredients, in a sterile manner or followed by sterilization (e.g., filter sterilization) after assembly.

In another embodiment, active agent-lipid particles can be prepared and incorporated into a broad range of lipid-containing dosage forms. For instance, the suspension containing the active agent-lipid particles can be formulated and administered as liposomes, gels, oils, emulsions, topical creams, pastes, ointments, lotions, foams, mousses, and the like.

In some embodiments, the active agents may be formulated in liposome compositions. Sterile aqueous solutions, active agent-lipid particles or dispersions comprising the active agent(s) are adapted for administration by encapsulation in liposomes. Such liposomal formulations can include an effective amount of the liposomally packaged active agent(s) suspended in diluents such as water, saline, or PEG 400.

The liposomes may be unilamellar or multilamellar and are formed of constituents selected from phosphatidylcholine, dipalmitoylphosphatidylcholine, cholesterol, phosphatidylethanolamine, phosphatidylserine, demyristoylphosphatidylcholine and combinations thereof. The multilamellar liposomes comprise multilamellar vesicles of similar composition to unilamellar vesicles but are prepared to provide a plurality of compartments in which the silver component in solution or emulsion is entrapped. Additionally, other adjuvants and modifiers may be included in the liposomal formulation such as polyethyleneglycol, or other materials.

While a suitable formulation of liposome includes dipalmitoyl-phosphatidylcholine:cholesterol (1:1) it is understood by those skilled in the art that any number of liposome bilayer compositions can be used in the composition of the present invention. Liposomes may be prepared by a variety of known methods such as those disclosed in U.S. Pat. No. 4,235,871 and in RRC, Liposomes: A Practical Approach. IRL Press, Oxford, 1990, pages 33-101.

The liposomes containing the active agents may have modifications such as having non-polymer molecules bound to the exterior of the liposome such as haptens, enzymes, antibodies or antibody fragments, cytokines and hormones and other small proteins, polypeptides or non-protein molecules which confer a desired enzymatic or surface recognition feature to the liposome. Surface molecules which preferentially target the liposome to specific organs or cell types include for example antibodies which target the liposomes to cells bearing specific antigens. Techniques for coupling such molecules are available (see for example U.S. Pat. No. 4,762,915 the disclosure of which is incorporated herein by reference). Alternatively, or in conjunction, one skilled in the art would understand that any number of lipids bearing a positive or negative net charge may be used to alter the surface charge or surface charge density of the liposome membrane. The liposomes can also incorporate thermal sensitive or pH sensitive lipids as a component of the lipid bilayer to provide controlled degradation of the lipid vesicle membrane.

Liposome formulations for use with active agents may also be formulated as disclosed in WO 2005/105152 (the disclosure of which is incorporated herein in its entirety). Briefly, such formulations comprise phospholipids and steroids as the lipid component. These formulations help to target the molecules associated therewith to in vivo locations without the use of an antibody or other molecule.

Antibody-conjugated liposomes, termed immunoliposomes, can be used to carry active agent(s) within their aqueous compartments. Compositions of active agent(s) provided within antibody labeled liposomes (immunoliposomes) can specifically target the active agent(s) to a particular cell or tissue type to elicit a localized effect. Methods for making of such immunoliposomal compositions are available, for example, in Selvam M. P., et al., 1996. Antiviral Res. December;33(1):11-20 (the disclosure of which is incorporated herein in its entirety).

For example, immunoliposomes can specifically deliver active agents to the cells possessing a unique antigenic marker recognized by the antibody portion of the immunoliposome Immunoliposomes are ideal for the in vivo delivery of active agent(s) to target tissues due to simplicity of manufacture and cell-specific specificity.

Muscle cell-specific antibodies, fat-cell specific antibodies, liver-cell specific antibodies, and other somatic cell-specific types of antibodies can be used in conjunction with the inhibitors or liposomes containing inhibitors to help target the inhibitors and liposomes to specific cell types. Other active agents can also be included in such liposomes.

In some instances, the active agents can be administered orally, in combination with a pharmaceutically acceptable vehicle such as an inert diluent or an assimilable edible carrier. They may be enclosed in hard or softshell gelatin capsules, may be compressed into tablets, or may be incorporated directly with the food of the patient's diet. For oral therapeutic administration, they may be combined with one or more excipients and used in the form of ingestible tablets, buccal tablets, troches, capsules, elixirs, suspensions, syrups, wafers, and the like. Such compositions and preparations may contain at least 0.1% of active compound. The percentage of the compositions and preparations may, of course, be varied. The amount of compound in such therapeutically useful compositions is such that an effective dosage level will be obtained.

The active agents can also be incorporated into dosage forms such as tablets, troches, pills, and capsules. These dosage forms may also contain any of the following: binders such as gum tragacanth, acacia, corn starch or gelatin; excipients such as dicalcium phosphate; a disintegrating agent such as corn starch, potato starch, alginic acid and the like; a lubricant such as magnesium stearate; polymers such as cellulose-containing polymers (e.g., hydroxypropyl methylcellulose, methylcellulose, ethylcellulose), polyethylene glycol, poly-glutamic acid, poly-aspartic acid or poly-lysine; and a sweetening agent such as lactose or aspartame or a flavoring agent such as peppermint, oil of wintergreen, or cherry flavoring may be added.

Tablet formulations can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active agents in a flavoring or sweetener, e.g., as well as pastilles comprising the active agent(s) in an inert base, such as gelatin and glycerin or sucrose and acacia emulsions, gels, and the like containing carriers available in the art.

When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the active compounds and agents may be incorporated into sustained-release preparations and devices.

Useful solid carriers include finely divided solids such as talc, clay, microcrystalline cellulose, silica, alumina, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In some embodiments, one or more of the active agents are linked to polyethylene glycol (PEG). For example, one of skill in the art may choose to link an active agent to PEG to form the following pegylated drug.

Useful dosages of the active agents (e.g., Pip4k2c guide RNAs, Pip4k2c binding agents, Pip4k2c degrading agents) can be determined by comparing their in vitro activity, and in vivo activity in animal models, for example, as described herein. Methods for the extrapolation of effective dosages in mice, and other animals, to humans are available to the art; for example, see U.S. Pat. No. 4,938,949. The agents can be conveniently administered in unit dosage form.

The desired dose may conveniently be presented in a single dose or as divided doses administered at appropriate intervals, for example, as two, three, four or more sub-doses per day. The sub-dose itself may be further divided, for example, into a number of discrete loosely spaced administrations, such as multiple oral, intraperitoneal, or intravenous doses. For example, it can be desirable to administer the present compositions intravenously over an extended period, either by continuous infusion or in separate doses.

The therapeutically effective amount of the active agent(s) necessarily varies with the subject and the disease, disease severity, or physiological problem to be treated. As one skilled in the art would recognize, the amount can be varied depending on the method of administration. The amount of the active agent (e.g., inhibitor) for use in treatment will vary not only with the route of administration, but also the nature of the condition being treated and the age and condition of the patient and will be ultimately at the discretion of the attendant physician or clinician.

The pharmaceutical compositions of the invention can include an effective amount of at least one of the active agents of the invention, or two or more different agents of the invention (e.g., two or more Pip4k2c inhibitors, Pip4k2c guide RNAs, or Pip4k2c degrading agents). These compositions can also include a pharmaceutically effective carrier.

The pharmaceutical compositions of the invention can also include other active ingredients and therapeutic agents, for example, anti-diabetes agents, anti-inflammatory agents, analgesics, vitamins, and the like. It is also within the scope of the present invention to combine any of the methods and any of the compositions disclosed herein with conventional diabetes therapies and various drugs in order to enhance the efficacy of such methods and/or compositions. For example, methods and compositions containing combinations of active agents can act through different mechanisms to improve the efficacy or speed of treatment. Methods and compositions containing combinations of active agents can also reduce the doses/toxicity of conventional therapies and/or to increase the sensitivity of conventional therapies.

For example, a variety of pharmaceutical preparations of insulin or diabetes medications can be used in combination with the methods and compositions described herein. For example, any of the following can be used with the methods and compositions described herein in the treatment of diabetes, such as regular insulin (such as Actrapid®), isophane insulin (designated NPH), insulin zinc suspensions (such as Semilente®, Lente®, and Ultralente®), and biphasic isophane insulin (such as NovoMix®). Human insulin analogues and derivatives have also been developed, designed for particular profiles of action, i.e. fast action or prolonged action. The long-acting insulin analogue, degludec (Begin™), as well as a biphasic preparation of degludec and the fast-acting insulin aspart, DegludecPlus (BOOST™), may be used. Some of the commercially available insulin preparations comprising rapid acting insulin analogues include NovoRapid® (preparation of B28Asp human insulin), Humalog® (preparation of B28LysB29Pro human insulin) and Apidra® (preparation of B3LysB29Glu human insulin). Some of the commercially available insulin preparations comprising long-acting insulin analogues include Lantus® (preparation of insulin glargine) and Levemir® (preparation of insulin detemir).

Monoclonal antibodies, nucleic acid inhibitors, and gene therapy are targeted therapies that can also be combined into the Pip4k2c compositions and used in the methods described herein. For example, such therapies can target myeloid cells, myeloid progenitor cells, basophils, neutrophils, eosinophils, monocytes, macrophages, dendritic cells, granulocytes, megakaryocytes, or any combination thereof.

The ultimate dosage form should be sterile, fluid, and stable under the conditions of manufacture and storage.

### Cell Therapies

In some cases, cells can be modified in vitro and then administered to a subject. For example, cells can be contacted and/or treated with any of the Pip4k2c degrading agents, Pip4k2c inhibitors, Pip4k2c mutating agents (e.g., guide RNAs), and/or other Pip4k2c modifying agents described herein. The cells can be autologous or allogeneic to the subject so administered. For example, the cells can be obtained from a subject, then these cells can be contacted and/or treated with any of the Pip4k2c degrading agents, Pip4k2c inhibitors, Pip4k2c mutating agents (e.g., guide RNAs), and/or other Pip4k2c modifying agents described herein to generate modified cells.

The modified cells can be expanded in culture to form a population of modified cells and the population of cells can be administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a device or a vehicle so that a population of liposomes, exosomes or microvesicles.

Cells are administered to patients at various time points to retard or inhibit tumor growth. Administration of cells should improve the immune status of the patient and reduce their risk of infections. Treatment may comprise the cells administered alone or with any Pip4k2c degrading agents, Pip4k2c inhibitors, Pip4k2c mutating agents (e.g., guide RNAs), and/or other Pip4k2c modifying agents described herein. Such agents can be administered separately from or with the modified cells. For example, the modified cells may be administered prior to, during, or after administering any of the Pip4k2c degrading agents, Pip4k2c inhibitors, Pip4k2c mutating agents (e.g., guide RNAs), and/or other Pip4k2c modifying agents described herein.

### Kits

Another aspect of the invention is one or more kits for modifying, inhibiting, or degrading Pip4k2c. For example, such kits can be used to generate agents to detect or treat cancer.

The kits of the present invention can include one or more modified cells, Pip4k2c inhibitor, Pip4k2c degrader, reagents for modifying genomic Pip4k2c sites, or other therapeutic reagents, or a combination thereof. The kits can also include instructions for making and/or administering the modified cells, Pip4k2c inhibitor, for degrading Pip4k2c, for modifying genomic Pip4k2c sites, or other therapeutic reagents.

In some cases, the kit can include reagents for isolating cells (e.g. myeloid cells, and/or other types of cells) from a subject and modifying genomic Pip4k2c sites therein. Such kits can include sterile implements for isolating cells from a subject, reagents for culturing cells, one or more guide RNA(s) for targeting genomic Pip4k2c sites, implements for administering modified cells back into the subject, and any combination thereof.

The following non-limiting Examples illustrate materials and methods used for development of the invention.

### Example 1: Materials and Methods

This Example illustrates some of the materials and methods employed in the development of the invention.

### Cell Lines, Authentication:

Cell lines were purchased from ATCC and/or fingerprinted with the

University of Arizona genetics core. Cells were tested to be mycoplasma free with Lonza Mycoalert.

### Cell Culture Conditions:

293T cells were cultured using DMEM media supplemented with 10% FBS, glutamine and pyruvate. H1299 and H1975 cells were cultured in RPMI media.

B16 and B16-F10 cells were maintained in RPMI-1640 (Wako) supplemented with 10% heat-inactivated FBS, 2 mmol/L L-glutamine, penicillin (100 U/mL), and streptomycin (100 μg/mL). MC38 cells were cultured using DMEM containing 9% heat-inactivated FCS, 2 mM glutamine, 2-ME, penicillin, and streptomycin.

Generation of Cell Lines with CRISPR Knockout of Pip4k2c:

CRISPR guides in pX458 were transfected in 293T cells. At 48-96 hours post transfection, GFP positive cells were single-cell sorted in 96-well plates using the Influx sorter at the WCMC Flow Cytometry Core. Two weeks later, wells were scored to contain single cell colony and expanded to screen for successful Pip4k2c knockout. Validation was performed by western blotting as well as PCR around each cut site.

TABLE 3
shRNA hairpin/CRISPR guide Sequences Targeting PIP4K Isoforms

| | | | Hairpin/ |
|---|---|---|---|
| Name | Target | Target Sequence | guide |
| C1406 | PIP4K2C | TTGAACATCAGAGAGAACCAGG | hairpin |
| | | (SEQ ID NO: 18) | |
| C3036 | PIP4K2C | TTCTAAAGAGCAATGGTTGCTG | hairpin |
| | | (SEQ ID NO: 19) | |
| C3165 | PIP4K2C | TATTATTATAGTAACAGGAGCA | hairpin |
| | | (SEQ ID NO: 20) | |
| g_PIP4K2C | PIP4K2C | ATCTGGCAGCAGCATCACCG(GGG) | guide |
| | | (SEQ ID NO: 21) | |

Pip4k2c knockout mice lines were also generated as illustrated in FIG. 11. In some experiments, a conditional Pip4k2c flox allele was introduced into immune cells (e.g., dendritic cells) that expressed cre to generate distinct Pip4k2c^{-/-} immune cell types.

### Example 2: Pip4k2c^{-/-} Mice Exhibit Profoundly Decreased Tumor Growth

Three tumor cell lines were selected for analysis of the effects of loss of Pip4k2c on tumor growth: the B16 cell line employed are melanoma cells, the MC38 cell line employed are colon carcinoma cells, and the KP1.9 cell line are lung adenocarcinoma cells. The B16 cells are more aggressive than the MC38 cells. For example, even deficiency of PD1 has little effect on B16 tumor growth.

Wild type and Pip4k2c knockout mice were injected subcutaneously with B16 cells, MC38 cells and the tumor growth was measured over time. Wild type and Pip4k2c knockout mice were injected intravenously with KP1.9 lung adenocarcinoma cells. Tumour burden of the KP1.9 lung adenocarcinoma cells was assessed by histological analyses of explanted lung tissue harvested 4 weeks post implantation.

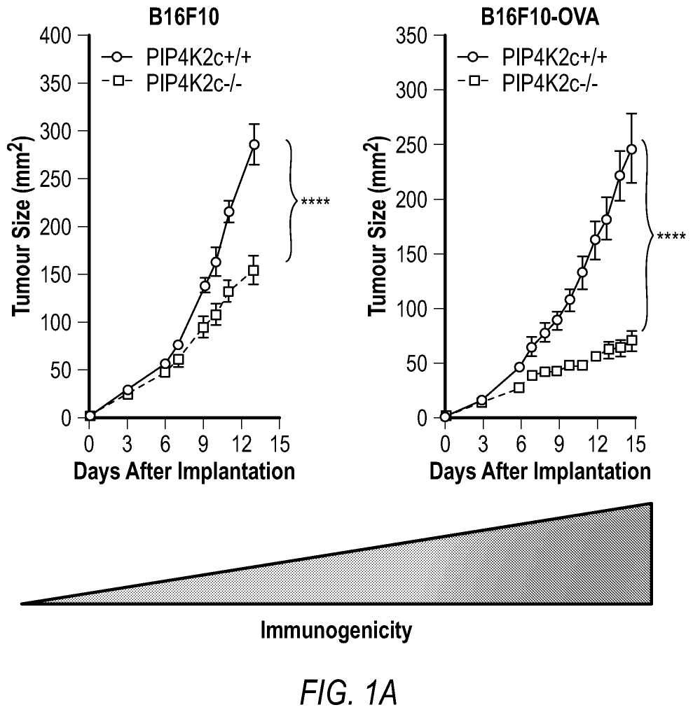

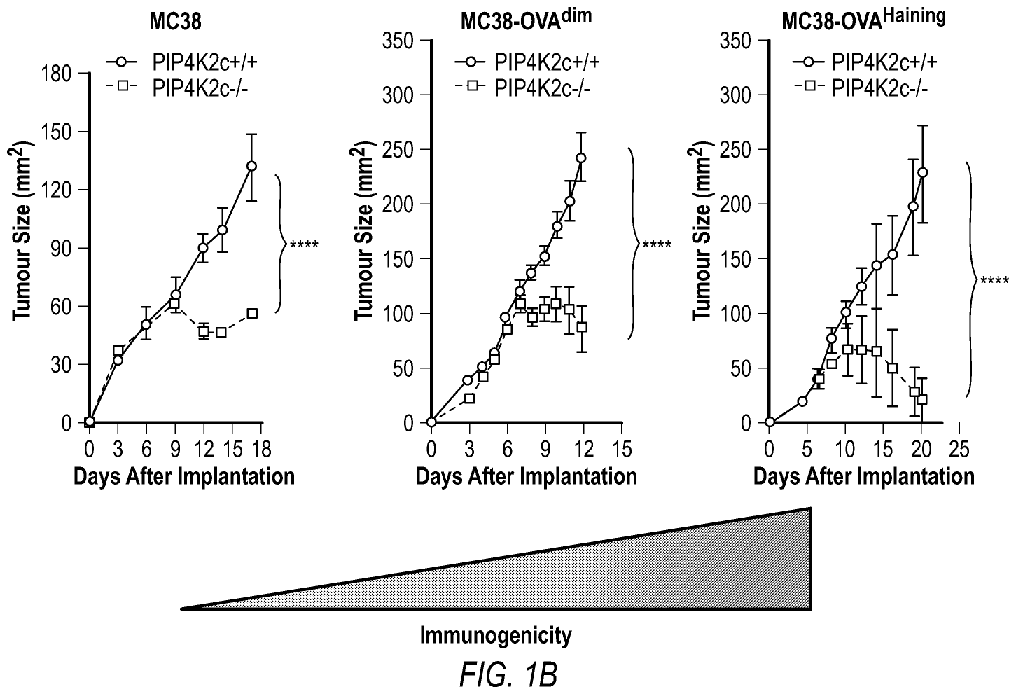

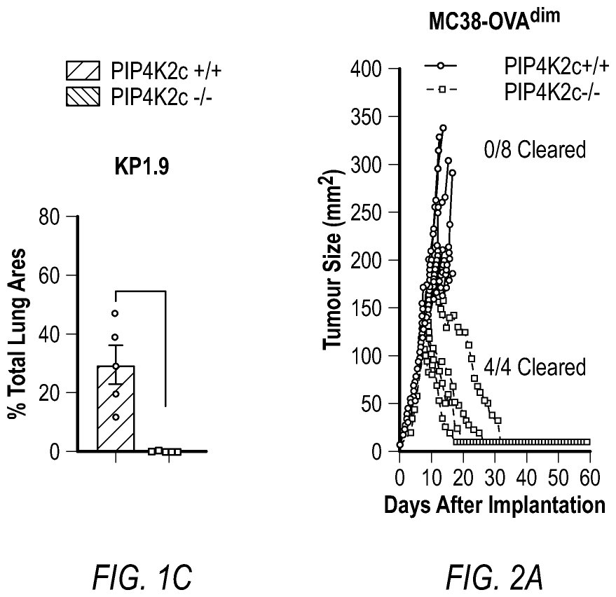

As shown in FIG. 1A-1C, tumor growth/tumor burden in Pip4k2c^{-/-} mice was substantially reduced compared to tumor growth/tumor burden in wild type Pip4k2c^{-/-} mice.

### Example 3: Pip4k2c^{-/-} Mice Develop Profound Memory Responses

This Example illustrates that loss of Pip4k2c improves immune memory responses.

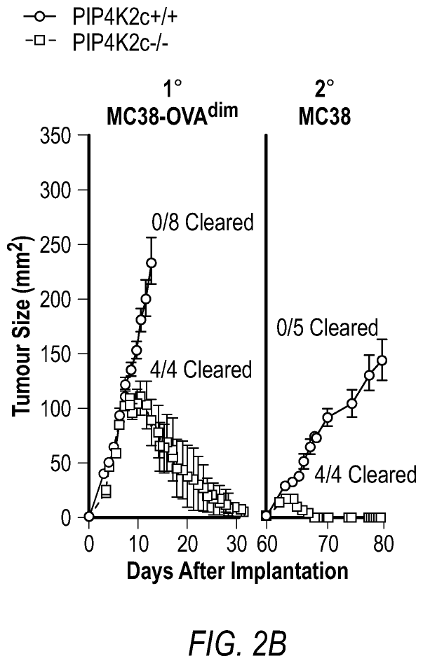

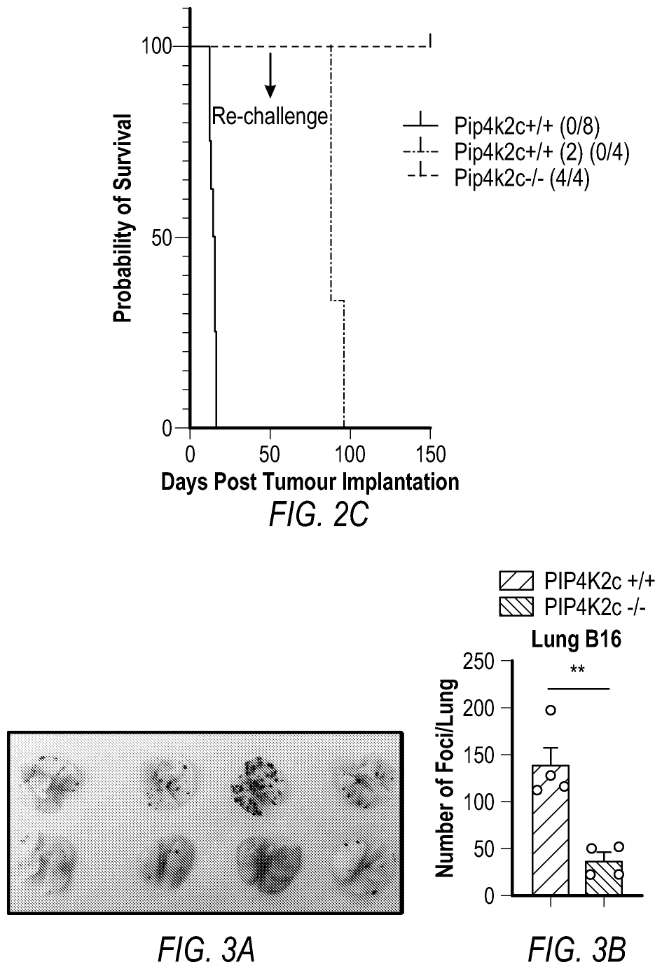

Wild type and Pip41(2c^{-/-} mice were injected subcutaneously with MC38 tumor cells that express an exogenous antigen-OVA. All of the Pip4k2c^{-/-} mice completely rejected the tumors, but the wild type mice did not (FIG. 2A). The mice were then maintained for one month allow immunological memory formation.

The same wild type and Pip4k2c^{-/-} mice were then challenged with parental MC38 cells that did not express the ova antigen. The Pip4k2c^{-/-} mice quickly rejected these tumors also (FIG. 2B). As shown in FIG. 2C, the so-treated Pip41(2c^{-/-} mice exhibit substantial prolonged survival compared to similarly treated wild type mice.

These results indicate that the Pip4k2c^{-/-} mice develop profound memory responses to the initially administrated MC38 tumor cells. In addition, the memory cells developed by the Pip41(2c^{-/-} mice were capable of reacting to a variety of antigens initially presented by the tumor cells because, as illustrated in FIG. 2B, re-challenge of the parental cells without the OVA antigen are also quickly rejected.

### Example 4: Pip4k2c^{-/-} Mice Have Decreased Incidence and Sizes of Tumor Foci

This Example illustrates that Pip4k2c^{-/-} mice have significantly fewer tumor foci than wild type mice administered the same numbers of cancer cells.

Wild type and Pip4k2c^{-/-} mice were injected intravenously with B16 melanoma cells. Such administration is a model of lung metastasis.

As shown in FIG. 3A, fewer B16 cells engrafted within the lungs of Pip4k2c^{-/-} mice than in the lungs of the wild type mice. FIG. 3B also shows that the sizes of tumors in Pip4k2c^{-/-} mice are smaller than the tumors in the lungs of the wild type mice. Similar results were obtained in experiments using a lung tumor cell line derived from endogenous lung tumors.

### Example 5: The Pip4k2c^{-/-} Phenotype is Immune Cell-Driven

To assess if the tumor control observed in the Pip4k2c^{-/-} mice was due to changes within blood cells/immune cells, C57BL6 mice were irradiated and then administered either wild type bone marrow cells or Pip4k2c^{-/-} bone marrow cells. The animals were then left to recover for 6-8 weeks. The mice with wild type or Pip4k2c^{-/-} bone marrow were then injected intravenously with B16 melanoma cells that express the OVA antigen. Tumor growth was measured over time. Weight was assessed at cessation of the experiment.

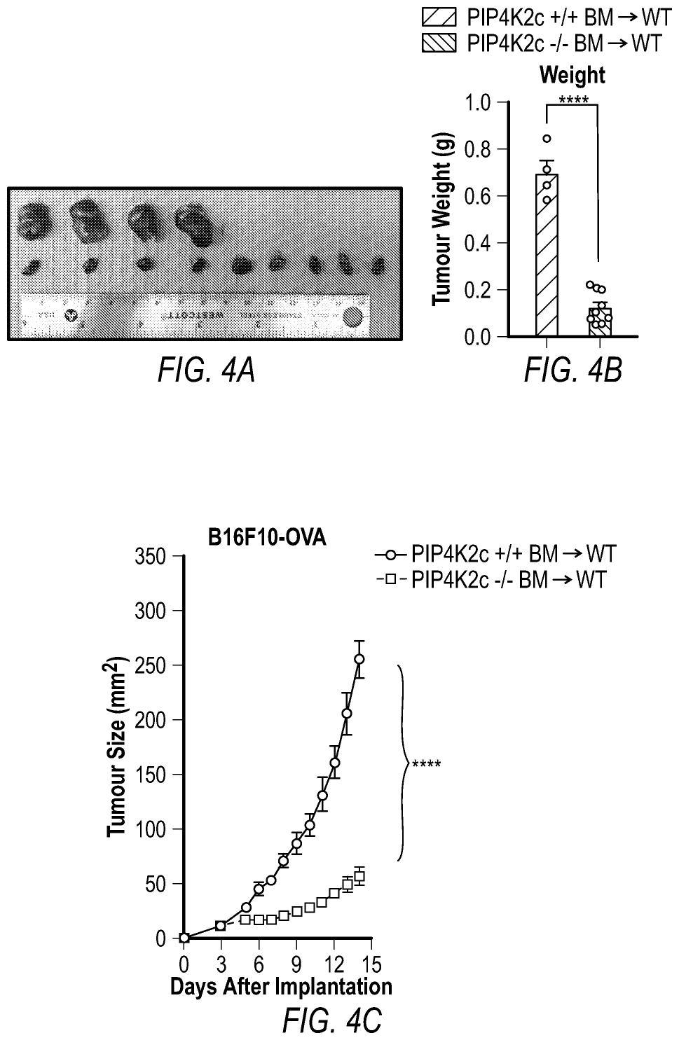

All recipients who received the Pip4k2c^{-/-} bone marrow had significantly less tumor growth than those who received WT bone marrow, indicating that the phenotype is due to loss of Pip4k2c within immune cells. This is shown in FIG. 4A-4C, where the numbers and sizes of tumors in mice receiving the Pip4k2c^{-/-} bone marrow were fewer and smaller than the tumors in the lungs of the mice receiving wild type bone marrow. Hence, the Pip4k2c^{-/-} phenotype is driven by hematopoietic cells.

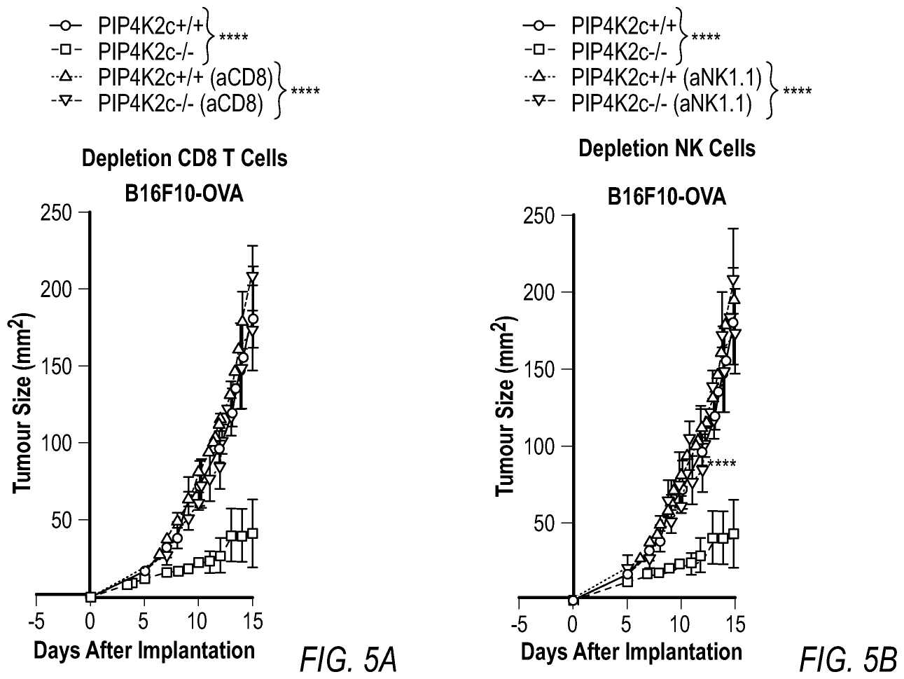

In a further experiment, wild type and Pip4k2c^{-/-} mice were injected subcutaneously with MC38 tumor cells that express an exogenous antigen-OVA. Some of the mice were treated with depleting antibodies to remove CD8 T cells or natural killer (NK) cells. As shown in FIG. 5A-5B, tumors were much smaller in Pip4k2c^{-/-} mice that were not treated with antibodies than in Pip4k2c^{-/-} mice that were treated with the antibodies. Hence, ablation of CD8 T cells or natural killer cells obviates the tumor-reductions seen in Pip4k2c^{-/-} mice, showing that Pip4k2c^{-/-} immune cells are responsible for the anti-tumor phenotype of Pip4k2c^{-/-} mice.

### Example 6: Global Deficiency of Pip4k2c Increases Immune Cells in Tumors

Wild type and Pip4k2c knockout mice were administered tumor cells. Fourteen days post-administration, the tumors were harvested and the absolute numbers of immune cells (total CD45⁺ leukocytes), CD4⁺ T cells, CD8+T cells and NK cells in the tumors from the Pip4k2c^{+/+} (WT) and Pip4k2c^{-/-} mice were determined by flow cytometry.

As shown in FIG. 6A-6D, there were significantly increased levels of CD45, natural killer cells, CD8 T cells, and CD4 T cells within immune infiltrates of tumors from Pip4k2c^{-/-} mice. Hence, Pip4k2c deficiency leads to increased immune cell infiltration into tumors.

Further analysis of immune cell infiltration by flow cytometry showed that tumors from Pip4k2c^{-/-} mice had increased numbers of CD4⁺ and CD8⁺ T cells (FIG. 6E-6G).

The results shown in FIG. 6E-6G demonstrate that there are significantly increased ratios of CD8:CD4 T cells in Pip4k2c^{-/-} mice. CD8 T cells are cytotoxic cells and higher ratios of CD8:CD4 cells correlate with better outcome for those suffering from cancer.

Major changes in frequencies of tumor infiltrating B Cells were also observed (data not shown).

### Example 7: CM⁺ Tumor Infiltrating Lymphocytes in Pip4k2c^{-/-} mice Express Higher Levels of Classic Exhaustion/Activation Markers

Flow cytometry analysis on cells from tumors of WT and Pip4k2c^{-/-} was used to assess the immune phenotypes of infiltrating CD8 T cells. As shown in FIG. 7, CD8 T cells from tumors of Pip4k2c^{-/-} mice appear to be highly activated.

The T cells isolated from Pip4k2c^{-/-} mice with tumors also express many markers associated with exhaustion, including PD1. Transient PD-1 cell surface expression is initiated upon T cell activation, but sustained expression is generally perceived to be a characteristic marker of T cell exhaustion. However, as illustrated in FIG. 8A-8D, Pip4k2c^{-/-} T cells from mice with tumors appear to more functional despite the elevated expression of many co-inhibitory molecules. For example, B16-OVA tumors from Pip4k2c^{-/-} mice and Pip4k2c^{-/-} T cell isolates have less CD160 than wild type T cell isolates (FIG. 8B). Note that CD160 is a marker of exhaustion/terminal differentiation that is expressed on functional NK and cytotoxic T lymphocytes.

These data indicate that combination of one or more PD1 activators and one or more Pip4k2c inhibitors/degraders would be useful for treatment of cancer.

T cells from Pip4k2c^{-/-} tumors were contacted ex vivo with the OVA peptide antigen and then evaluated by using flow cytometry to ascertain what types of functions are activated/expressed by the T cells. FIG. 9A shows that a higher proportion of CD8⁺ PD1⁺ T cells from Pip4k2c^{-/-} tumors are antigen (OVA) specific than the CD8⁺ PD1⁺ T cells from wild type tumors. As shown in FIG. 9B-9E, the Pip4k2c^{-/-} T cells were highly functional ex vivo, producing significantly more lytic enzymes than similarly treated wild type tumor T cells. For example, as shown in

FIG. 9B-9E, higher levels of perforin, interferon-gamma, granzyme B, and CD107a are expressed by Pip4k2c^{-/-} T cells than by similarly treated wild type T cells.

This increased functionality together with increased expression of inhibitory molecules such as PD1 and TIGIT shows that combination therapies may be the most effective for treatment of cancer such as those that include conventional checkpoint immunotherapies and Pip4k2c inhibition/degrader.

The numbers of different cell types were evaluated in wild type and Pip4k2c^{-/-} tumors using flow cytometric analysis. The results indicated that tumors from Pip4k2c^{-/-} mice exhibit significant remodeling of the tumor myeloid compartment.

As shown in FIG. 10A, Pip4k2c^{-/-} tumors have increased numbers of viable CD45⁺ cells compared to wild type tumors. FIG. 10B-10C show that Pip4k2c^{-/-} tumors have increased percentages of CD24⁻ CD11b⁺ myeloid cells such as monocytes and macrophages compared to wildype tumors. FIG. 10C also shows that Pip4k2c^{-/-} monocytes and macrophages express higher percentages of activation markers such as MHC class II molecules, CD86, and Tim3. In addition, the inventors observed that tumor infiltrating plasmacytoid derived dendritic cells (DC2s) exhibit increased expression of activation markers in Pip4k2c^{-/-} mice compared to wild type mice (data not shown).

### Example 8: Specific In Vivo Deletion of Pip4k2c in Immune Cell Types

To determine which cell type is contributing to the global phenotype and tumor control exhibited by Pip4k2c^{-/-} mice, a conditional Pip4k2c flox allele was crossed with many different immune specific cre lines to deplete Pip4k2c specifically in a particular cell type as shown in FIG. 11. Hence, rather than global Pip4k2c loss, different mouse lines were generated, each with a single distinct Pip4k2c^{-/-} immune cell type.

The mice lines with distinct Pip4k2c^{-/-} immune cell types were inoculated with B16OVA and the tumor sizes were measured over time.

As shown in FIG. 12A-12C there was no significant tumor growth inhibition in mice with conditional deletions of Pip4k2c in total T cells, B cells or natural killer (NK) cells. Hence, these conditional knockout Pip4k2c^{-/-} mice were able to rule out that Pip4k2c^{-/-} has an anti-tumor role in B cells, T cells and NK cells. These data show that loss of Pip4k2c in any of these cells is not what drives the anti-tumor phenotype of global Pip4k2c^{-/-} loss.

The inventors also observed that there was an increased percentage of Tregs in the global Pip4k2c^{-/-} mice with tumors (FIG. 13A). To further evaluate the role of Tregs in the Pip4k2c^{-/-} phenotype, Pip4k2c flox was crossed to an inducible cre with to provide specific deletion of Pip4k2c in Tregs upon administration of Tamoxifen. As shown in FIG. 13B, cell specific deletion of Pip4k2c in Tregs (by tamoxifen induction) did diminish tumor burden.

### Example 9: Loss of Pip4k2c in Dendritic Cells Reduces Tumor Burden

This Example illustrates that the most significant reduction in tumor sizes was observed when dendritic cells (DCs) were conditionally Pip4k2c-deleted using DC-specific cres. Pip4k2c flox was crossed to CD11c cre and later to Zbtb46cre, which is more specific. Wild type and mice with the Pip4k2c-deleted dendritic cells were inoculated with B160VA tumor cells and tumor growth was measured over time.

As shown in FIG. 14A-14C, mice with the Pip4k2c-deleted dendritic cells (Pip4k2c^{fl.fl}×CD11c and Pip4k2c^{fl.fl}×Zbtb46) exhibited reduced tumor sizes and reduced tumor weights compared to mice with wild type Pip4k2c.

Flow cytometry analysis was performed on tumor cells from wild type mice or on tumor cells from mice with the Pip4k2c-deleted dendritic cells. As shown in FIG. 15A-15B, deletion of Pip4k2c in dendritic cells leads to increased proportion of dendritic cells (DC1 and DC2) in CD45+ cells from tumor cells isolated from mice with the Pip4k2c-deleted dendritic cells compared to CD45+cells from wild type mice. Note that DC1 cells are important for anti-tumor immunity for many target tumors. FIG. 15C-15D show that the proportions of polymorphonuclear leukocytes (PMNs) and macrophages are reduced in CD45+ cells from tumor cells isolated from mice with the Pip4k2c-deleted dendritic cells compared to CD45+ cells from wild type mice. However, the proportion of monocytes in CD45+ cells from tumor cells isolated from mice with the Pip4k2c-deleted dendritic cells is higher compared to the proportion of monocytes in CD45+ cells from wild type mice.

### Example 10: Loss of Pip4k2c in Dendritic Cells Increases Their Functionality

In another experiment, cells were harvested from tumors generated by administration of B16-OVA into wild type mice or into mice with the Pip4k2c-deleted dendritic cells. The harvested cells were stimulated for 4 hours in the presence of Brefeldin and Monensin followed by cytokine and chemokine analysis by intracellular staining. The different cell types were detected/quantified by flow cytometry.

As shown in FIG. 16, the increased numbers of DC1 cells in mice with the Pip4k2c-deleted dendritic cells exhibit increased functionality in tumors, including increases in IL12b40, IL27p28, CXCL16 and CXCL9.

In another experiment, CD4+cells were harvested from tumors generated from B16-OVA cells in wild type mice or in mice with the Pip4k2c-deleted dendritic cells. The cells were stained for PD1, Tim3, CD69 and KLRG1 to identify different T cell populations. T cell immunoglobulin and mucin domain-containing protein 3 (TIM3) is a member of the TIM family, and is a receptor expressed on interferon-y-producing CD4+and CD8+T cells. Programmed cell death protein 1, also known as PD-1 and CD279 (cluster of differentiation 279), is a protein on the surface of T and B cells. CD69 (Cluster of Differentiation 69) is a human transmembrane C-Type lectin protein that is an early activation marker expressed in hematopoietic stem cells, T cells, and other cell types in the immune system. The KLRG1 protein belongs to the killer cell lectin-like receptor (KLR) family of proteins, which are transmembrane proteins preferentially expressed in NK cells.

FIG. 17 illustrates that CD4 T helper cells from mice with the Pip4k2c-deleted dendritic cells are less exhausted, as indicated by lower expression levels of PD1 and KLRG1 compared to CD4 T helper cells from wild type mice. CD4 T cells help CD8 cells and can become dysfunctional just like exhausted CD8 T cells. However, loss of Pip4k2c in dendritic cells can avoid or reduce the incidence and/or severity of exhaustion in CD4 T cells.

In addition, a similar experiment where expression of transcription factors associated with effector T cell responses. As shown in FIG. 18A-18B, expression levels of Tbet and Ki67 were increased amongst the population of CD4⁺Foxp3⁻ cells in mice with Pip4k2c-deleted tumors, indicating that loss of Pip4k2c can enhance effector T cell responses. Hence, CD4 T cells from Pip4k2c-deleted tumors are likely to be better helper cells with improved effector function.

In another experiment, cells were harvested from B16-0VA tumors of mice with the Pip4k2c-deleted dendritic cells or from B16-OVA tumors of wild type mice. The harvested cells were stimulated for 4 hours in the presence of Brefeldin and Monensin followed by cytokine analysis detected by intracellular staining. Cells were then evaluated flow cytometry. As shown in FIG. 19, the CD4 T cells from B16-OVA tumors of mice with the Pip4k2c-deleted dendritic cells produced more effector cytokines such as IFNγ and IL-2. Such cytokines can re-model the tumor microenvironment and help sustain CD8 T cell responses.

In another experiment, cells were harvested from tumors of mice with the Pip4k2c-deleted dendritic cells or from B16-OVA tumors of wild type mice. The harvested cells were stimulated for 4 hours in the presence of Brefeldin and Monensin and analyzed for cytokine expression by intracellular staining and flow cytometry.

As shown in FIG. 20A-20F, the CD8 T cells in tumors from mice with the Pip4k2c-deleted dendritic cells are more potent killer cells, expressing increased levels of lytic molecules such as granzyme B, perforin, CD107a, interleukin-2, and interferon-γ.

An overview of the tumor landscape when Pip4k2c is deleted or inhibited in dendritic cells is shown in FIG. 21.

When a subject has cancer, insulin resistance/insensitivity can arise in tumor infiltrating lymphocytes (TILs), promoting immune cell dysfunction, and blunting anti-tumor immune responses. However, by inhibiting, degrading, or deleting Pip4k2c, especially in dendritic cells, such insulin resistance can be mitigated.

To optimize the anti-tumor effects of Pip4k2c reduction, therapeutic strategies can be employed that combine Pip4k2c inhibition/degradation/deletion, anti-PD1 therapeutic agents, and/or chemotherapeutic agents with targeting agents.

### Example 11: Pip4k2c Deficiency does not Lead to Significant Changes in Immune Cell Populations at Homeostasis

This Example illustrates that the numbers of immune cells in Pip4k2c^{-/-} mice at homeostasis are substantially similar to those in wild type mice.

Transcript levels of Pip4k2c were evaluated in Pip4k2c^{-/-} mice. As illustrated in FIG. 22A, qPCR analysis shows that percent of Pip4k2c transcripts releative to housekeeping gene (HKG) transcripts is significantly reduced in Pip4k2c^{-/-} mice compared to wild type mice. Hence, knockout of the Pip4k2c gene in mice significantly depresses Pip4k2c expression.

Flow cytometry of live CD45+ cells from thymus, spleen and lymph nodes from wild type (Pip4k2c+/+) and Pip4k2c knockout (Pip4k2c−/−) mice was performed to evaluate the percentages of immune cells present in these mice.

As shown in FIG. 22B, the absolute number of live CD45+ cells in the total number of leukocytes within wild type (Pip4k2c+/+) mice versus Pip4k2c knockout (Pip4k2c−/−) mice was not significantly different. However, the absolute number of live CD45+cells in total leukocytes within the spleens and lymph nodes of wild type (Pip4k2c+/+) mice was slightly higher (though not significantly different from) than the number of live CD45+cells in total leukocytes observed the spleens and lymph nodes of Pip4k2c knockout (Pip4k2c−/−) mice.

FIG. 22C shows that the percentages of live CD45+ cells that are T cells, B cells and NK cells in thymus, spleen and lymph nodes are similar in WT (Pip4k2c+/+) and Knockout (Pip4k2c−/−) mice as detected by flow cytometry analysis. FIG. 22D shows that the percentages of live CD45+cells that are various types of myeloid cells including neutrophils, monocytes, macrophages and DCs in thymus, spleen and lymph nodes are also similar in WT (Pip4k2c+/+) and KO (Pip4k2c−/−) mice as detected by flow cytometry.

### Example 12: Loss of Pip4k2c Reduces Tumor Burden

Melanoma B16 cell lines were generated with specific deletions of Pip4k2c using CRISPR-Cas9 and Pip4k2c-specific guide RNAs to generate Pip4k2c^{-/-} (sgPip4k2c) cells. As a control, melanoma cells were treated with a scrambled, non-specific guide RNA (sgScramble).

Lysates were generated from control (sgScramble) and Pip4k2c^{-/-} (sgPip4k2c) B16 cell lines, and western blots were used to detect expression of Pip4k2c, Pip4k2a, Pip4k2b, where beta-actin was used as a loading control.

As shown in FIG. 23A, expression of Pip4k2c was dramatically reduced in the sgPip4k2c B16 cell line compared to the sgScramble control cells, but Pip4k2a and Pip4k2b expression levels were not reduced in the sgPip4k2c B16 cells.

The sgPip4k2c B16 tumor cells were implanted into wild type mice and tumor growth was monitored over time. As shown in FIG. 23B, tumor sizes of the sgPip4k2c B16 tumor cells exhibited reduced growth (size) compared to the sgScramble control cells. FIG. 23C illustrates tumor weight (in grams) as a measure of tumor burden.

The sgPip4k2c B16 tumor cells were implanted into immunodeficient NSG mice and tumor growth was monitored over time. As shown in FIG. 23D, sgPip4k2c B16 tumor cells in the immunodeficient NSG mice exhibited reduced growth (size) compared to the sgScramble control tumor cells.

These results indicate that loss of Pip4k2c leads reduced tumor burden.

### Example 13: Administration of Pip4k2c Deficient DCs Leads to Protective Anti-Tumor Immunity

This Example illustrates that tumor sizes are smaller in mice administered dendritic cells having deleted/knocked out Pip4k2c.

Wild type mice were implanted with B16OVA tumors and these mice were randomized to receive PBS, OVA pulsed WT DC1, or OVA pulsed DC1. This procedure and the timing of dendritic cells (DC1) is illustrated in FIG. 24A.

As shown in FIG. 24B, mice receiving Pip4k2c^{-/-} DC1 exhibited significantly reduced tumor growth over time.

### REFERENCES

Balla, T. (2013). Phosphoinositides: tiny lipids with giant impact on cell regulation. Physiol Rev 93, 1019-1137.
Bondeson, D. P., and Crews, C. M. (2017). Targeted Protein Degradation by Small Molecules. Annu Rev Pharmacol Toxicol 57, 107-123.
Brown, D. A. (2015). PIP2Clustering: From model membranes to cells. Chem Phys Lipids 192, 33-40.
Bultsma, Y., Keune, W. J., and Divecha, N. (2010). PIP4Kbeta interacts with and modulates nuclear localization of the high-activity PtdIns5P-4-kinase isoform PIP4Kalpha. Biochem J 430, 223-235.
Carricaburu, V., Lamia, K. A., Lo, E., Favereaux, L., Payrastre, B., Cantley, L. C., and Rameh, L. E. (2003). The phosphatidylinositol (PI)-5-phosphate 4-kinase type II enzyme controls insulin signaling by regulating PI-3,4,5-trisphosphate degradation. Proc Natl Acad Sci USA 100, 9867-9872.
Choi, S., Hedman, A. C., Sayedyahossein, S., Thapa, N., Sacks, D. B., and Anderson, R. A. (2016). Agonist-stimulated phosphatidylinositol-3,4,5-trisphosphate generation by scaffolded phosphoinositide kinases. Nat Cell Biol 18, 1324-1335.
Choi, S., Thapa, N., Tan, X., Hedman, A. C., and Anderson, R. A. (2015). PIP kinases define PI4,5P(2)signaling specificity by association with effectors. Biochim Biophys Acta 1851, 711-723.
Clarke, J. H., Emson, P. C., and Irvine, R. F. (2008). Localization of phosphatidylinositol phosphate kinase IIgamma in kidney to a membrane trafficking compartment within specialized cells of the nephron. Am J Physiol Renal Physiol 295, F1422-1430.
Clarke, Jonathan H., and Irvine, Robin F. (2013). Evolutionarily conserved structural changes in phosphatidylinositol 5-phosphate 4-kinase (PI5P4K) isoforms are responsible for differences in enzyme activity and localization. Biochemical Journal 454, 49-57.
De Leo, M. G., Staiano, L., Vicinanza, M., Luciani, A., Carissimo, A., Mutarelli, M., Di Campli, A., Polishchuk, E., Di Tullio, G., Morra, V., et al. (2016). Autophagosome-lysosome fusion triggers a lysosomal response mediated by TLR9 and controlled by OCRL. Nat Cell Biol 18, 839-850.
Emerling, B. M., Hurov, J. B., Poulogiannis, G., Tsukazawa, K. S., Choo-Wing, R., Wulf, G. M., Bell, E. L., Shim, H. S., Lamia, K. A., Rameh, L. E., et al. (2013). Depletion of a putatively druggable class of phosphatidylinositol kinases inhibits growth of p53-null tumors. Cell 155, 844-857.
Fellmann, C., Hoffmann, T., Sridhar, V., Hopfgartner, B., Muhar, M., Roth, M., Lai, D. Y., Barbosa, I. A., Kwon, J. S., Guan, Y., et al. (2013). An optimized microRNA backbone for effective single-copy RNAi. Cell Rep 5, 1704-1713.
Fellmann, C., Zuber, J., McJunkin, K., Chang, K., Malone, C. D., Dickins, R. A., Xu, Q., Hengartner, M. O., Elledge, S. J., Hannon, G. J., et al. (2011). Functional identification of optimized RNAi triggers using a massively parallel sensor assay. Mol Cell 41, 733-746.
Fruman, D. A., Chiu, H., Hopkins, B. D., Bagrodia, S., Cantley, L. C., and Abraham, R. T. (2017). The PI3K Pathway in Human Disease. Cell 170, 605-635.
Guo, J., Wenk, M. R., Pellegrini, L., Onofri, F., Benfenati, F., and De Camilli, P (2003). Phosphatidylinositol 4-kinase type IIalpha is responsible for the phosphatidylinositol 4-kinase activity associated with synaptic vesicles. Proc Natl Acad Sci USA 100, 3995-4000.
Gupta, A., Toscano, S., Trivedi, D., Jones, D. R., Mathre, S., Clarke, J. H., Divecha, N., and Raghu, P. (2013). Phosphatidylinositol 5-phosphate 4-kinase (PIP4K) regulates TOR signaling and cell growth during Drosophila development. Proc Natl Acad Sci USA 110, 5963-5968.
Hinchliffe, K.., Ciruela, A., Letcher, A.., Divecha, N., and Irvine, R.F. (1999). Regulation of type IIalpha phosphatidylinositol phosphate kinase localisation by the protein kinase CK2. Curr Biol 9, 983-986.
Hinchliffe, K. A., Giudici, M. L., Letcher, A. J., and Irvine, R. F. (2002). Type Ha phosphatidylinositol phosphate kinase associates with the plasma membrane ia interaction with type I isoforms. Biochem J 363, 563-570.
Homma, K., Terui, S., Minemura, M., Qadota, H., Anraku, Y., Kanaho, Y., and Ohya, Y. (1998). Phosphatidylinositol-4-phosphate 5-kinase localized on the plasma membrane is essential for yeast cell morphogenesis. Journal of Biological Chemistry 273, 15779-15786.
Huttlin, E. L., Bruckner, R. J., Paulo, J. A., Cannon, J. R., Ting, L., Baltier, K., Colby, G., Gebreab, F., Gygi, M. P., Parzen, H., et al. (2017). Architecture of the human interactome defines protein communities and disease networks. Nature 545, 505-509.
Idevall-Hagren, O., and De Camilli, P (2015). Detection and manipulation of phosphoinositides. Biochim Biophys Acta 1851, 736-745.
Itoh, T., Ijuin, T., and Takenawa, T. (1998). A novel phosphatidylinositol-5-phosphate 4-kinase (phosphatidylinositol-phosphate kinase IIgamma) is phosphorylated in the endoplasmic reticulum in response to mitogenic signals. J Biol Chem 273, 20292-20299.
Itzhak, D. N., Tyanova, S., Cox, J., and Borner, G. H. (2016). Global, quantitative and dynamic mapping of protein subcellular localization. Elife 5.
Jefferies, H. B., Cooke, F. T., Jat, P., Boucheron, C., Koizumi, T., Hayakawa, M., Kaizawa, H., Ohishi, T., Workman, P., Waterfield, M. D., et al. (2008). A selective PIKfyve inhibitor blocks PtdIns(3,5)P(2) production and disrupts endomembrane transport and retroviral budding. EMBO Rep 9, 164-170.
Jenkins, G. H., Fisette, P. L., and Anderson, R. A. (1994). Type I phosphatidylinositol 4-phosphate 5-kinase isoforms are specifically stimulated by phosphatidic acid. J Biol Chem 269, 11547-11554.
Jones, D. R., Bultsma, Y., Keune, W. J., Halstead, J. R., Elouarrat, D., Mohammed, S., Heck, A. J., D'Santos, C. S., and Divecha, N. (2006). Nuclear PtdIns5P as a transducer of stress signaling: an in vivo role for PIP4Kbeta. Mol Cell 23, 685-695.
Kunz, J., Fuelling, A., Kolbe, L., and Anderson, R. A. (2001). Stereo-specific Substrate Recognition by Phosphatidylinositol Phosphate Kinases Is Swapped by Changing a Single Amino Acid Residue. Journal of Biological Chemistry 277, 5611-5619.
Kunz, J., Wilson, M. P., Kisseleva, M., Hurley, J. H., Majerus, P. W., and Anderson, R .A. (2000a). The Activation Loop of Phosphatidylinositol Phosphate Kinases Determines Signaling Specificity. Mol Cell 5, 1-11.
Kunz, J., Wilson, M. P., Kisseleva, M., Hurley, J. H., Majerus, P. W., and Anderson, R. A. (2000b). The activation loop of phosphatidylinositol phosphate kinases determines signaling specificity. Mol Cell 5, 1-11.
Lamia, K. A., Peroni, O. D., Kim, Y. B., Rameh, L. E., Kahn, B. B., and Cantley, L. C. (2004). Increased insulin sensitivity and reduced adiposity in phosphatidylinositol 5-phosphate 4-kinase beta−/− mice. Mol Cell Biol 24, 5080-5087.
Lundquist, M. R., Goncalves, M. D., Loughran, R. M., Possik, E., Vijayaraghavan, T., Yang, A., Pauli, C., Ravi, A., Verma, A., Yang, Z., et al. (2018). Phosphatidylinositol-5-Phosphate 4-Kinases Regulate Cellular Lipid Metabolism By Facilitating Autophagy. Molecular Cell 70, 531-544.e539.
Mackey, A. M., Sarkes, D. A., Bettencourt, I., Asara, J. M., and Rameh, L. E. (2014). PIP4kgamma is a substrate for mTORC1 that maintains basal mTORC1 signaling during starvation. Sci Signal 7, ra104.
Manning, B. D., and Toker, A. (2017). AKT/PKB Signaling: Navigating the Network. Cell 169, 381-405.
Muftuoglu, Y., Xue, Y., Gao, X., Wu, D., and Ha, Y. (2016). Mechanism of substrate specificity of phosphatidylinositol phosphate kinases. Proc Natl Acad Sci U S A 113, 8711-8716.
Nakatsu, F., Baskin, J. M., Chung, J., Tanner, L. B., Shui, G., Lee, S. Y., Pirruccello, M., Hao, M., Ingolia, N. T., Wenk, M. R., et al. (2012). PtdIns4P synthesis by PI4KIIIalpha at the plasma membrane and its impact on plasma membrane identity. J Cell Biol 199, 1003-1016.
Niebuhr, K., Giuriato, S., Pedron, T., Philpott, D. J., Gaits, F., Sable, J., Sheetz, M. P., Parsot, C., Sansonetti, P. J., and Payrastre, B. (2002). Conversion of PtdIns(4,5)P(2) into Ptdlns(5)P by the S.flexneri effector IpgD reorganizes host cell morphology. EMBO J 21, 5069-5078.
Pelossof, R., Fairchild, L., Huang, C. H., Widmer, C., Sreedharan, V. T., Sinha, N., Lai, D. Y., Guan, Y., Premsrirut, P. K., Tschaharganeh, D. F., et al. (2017). Prediction of potent shRNAs with a sequential classification algorithm. Nat Biotechnol 35, 350-353.
Pendaries, C., Tronchere, H., Arbibe, L., Mounier, J., Gozani, 0., Cantley, L. C., Fry, M. J., Gaits-Iacovoni, F., Sansonetti, J., and Payrastre, B. (2006). Ptdlns(5)P activates the host cell PI3-kinase/Akt pathway during Shigella flexneri infection. EMBO J 25, 1024-2034.
Rameh, L. E., Tolias, K. F., Duckworth, B. C., and Cantley, L. C. (1997). A new pathway for synthesis of phosphatidylinositol-4,5-bisphosphate. Nature 390, 192-196.
Ran, F. A., Hsu, P. D., Wright, J., Agarwala, V., Scott, D. A., and Zhang, F. (2013). Genome engineering using the CRISPR-Cas9 system. Nat Protoc 8, 2281-2308.
Saito, K., Tolias, K. F., Saci, A., Koon, H. B., Humphries, L. A., Scharenberg, A., Rawlings, D .J., Kinet, J. P., and Carpenter, C. L. (2003). BTK regulates PtdIns-4,5-P2 synthesis: importance for calcium signaling and PI3K activity. Immunity 19, 669-678.
Shim, H., Wu, C , Ramsamooj, S., Bosch, K. N., Chen, Z., Emerling, B. M., Yun, J., Liu, H., Choo-Wing, R., Yang, Z., et al. (2016). Deletion of the gene Pip4k2c, a novel phosphatidylinositol kinase, results in hyperactivation of the immune system. Proc Natl Acad Sci USA 113, 7596-7601.
Sun, Y., Thapa, N., Hedman, A. C., and Anderson, R .A. (2013). Phosphatidylinositol 4,5-bisphosphate: targeted production and signaling. Bioessays 35, 513-522.
van den Bout, I., and Divecha, N. (2009). PIPSK-driven Ptdlns(4,5)P2 synthesis:

regulation and cellular functions. J Cell Sci 122, 3837-3850.
Wang, H., Sun, H.-Q., Zhu, X., Zhang, L., Albanesi, J., Levine, B., and Yin, H. (2015). GABARAPs regulate PI4P-dependent autophagosome:lysosome fusion. Proceedings of the National Academy of Sciences, 201507263.
Weernink, P. A., Meletiadis, K., Hommeltenberg, S., Hinz, M., Ishihara, H., Schmidt, M., and Jakobs, K. H. (2004). Activation of type I phosphatidylinositol 4-phosphate 5-kinase isoforms by the Rho GTPases, RhoA, Rac1, and Cdc42. J Biol Chem 279, 7840-7849.
Wilcox, A., and Hinchliffe, K. A. (2008). Regulation of extranuclear Ptdlns5P production by phosphatidylinositol phosphate 4-kinase 2alpha. FEBS Lett 582, 1391-1394.
Wisniewski, J. R., Hein, M. Y., Cox, J., and Mann, M. (2014). A “proteomic ruler” for protein copy number and concentration estimation without spike-in standards. Mol Cell Proteomics 13, 3497-3506.
Xie, Z., Chang, S. M., Pennypacker, S. D., Liao, E. Y., and Bikle, D. D. (2009). Phosphatidylinositol-4-phosphate 5-kinase lalpha mediates extracellular calcium-induced keratinocyte differentiation. Mol Biol Cell 20, 1695-1704.
Zolov, S. N., Bridges, D., Zhang, Y., Lee, W. W., Riehle, E., Verma, R., Lenk, G. M., Converso-Baran, K., Weide, T., Albin, R. L., et al. (2012). In vivo, Pikfyve generates PI(3,5)P2, which serves as both a signaling lipid and the major precursor for PISP. Proc Natl Acad Sci USA 109, 17472-17477.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various aspects of the invention according to the foregoing description in the specification.

### Statements:

1. A cell comprising a deletion, substitution, or insertion in an endogenous Pip4k2c gene.
2. The cell of statement 1, wherein the deletion, substitution, or insertion reduces or eliminates transcription and/or translation of Pip4k2c from the Pip4k2c gene.
3. The cell of statement 1 or 2, wherein the cell is a myeloid cell, myeloid progenitor cell, lymphocyte, regulatory T cell, dendritic cell, bone marrow cell, granulocyte, basophil, eosinophil, neutrophil, monocyte, mast cell, megakaryocyte, erythrocyte, macrophage, platelet, tumor cell, malignant cell or a combination thereof.
4. The cell of statement 1, 2 or 3, wherein the cell is autologous or allogeneic to a mammalian subject.
5. A composition comprising a population of the cells of statement 1-3, or 4.
6. A method comprising administering the cell of statement 1-3 or 4, or the composition of statement 5 to a subject.
7. The method of statement 6, wherein the subject has cancer or an immune condition or immune disease.
8. The method of statement 6 or 7, wherein the subject is a human or a warm-blooded animal
9. The method of statement 6, 7 or 8, wherein the cell is one or more myeloid cells, lymphocytes, regulatory T cells, dendritic cells, bone marrow cells, granulocytes, basophils, eosinophils, neutrophils, monocytes, mast cells, erythrocytes, macrophages, platelets, tumor cells, malignant cells, or a combination thereof.
10. A composition comprising a Pip4k2c modifying agent and a carrier.
11. The composition of statement 10, wherein the carrier comprises a particle displaying an antibody or binding moiety that specifically binds to a cell via a cell surface marker.
12. The composition of statement 10 or 11, wherein the carrier comprises particles, nanoparticles, liposomes, beads, proteins, polysaccharides, lipids, or combinations thereof.
13. The composition of statement 10, 11 or 12, wherein the carrier can target the Pip4k2c modifying agent to a myeloid cell, myeloid progenitor cell, lymphocyte, regulatory T cell, dendritic cell, bone marrow cell, granulocyte, basophil, eosinophil, neutrophil, monocyte, mast cell, megakaryocyte, erythrocyte, macrophage, platelet, tumor cell, malignant cell or a combination thereof.
14. The composition of statement 10-12 or 13, wherein the carrier comprises a binding agent for one or more of the following cell surface markers mannose receptor (CD206), aminopeptidase N/CD13, CCR2, CCR3, CD11b/Integrin alpha M, CD14, CD34, CD36/SR-B3, CD38, CD44, CD59, CD68/SR-D1, CD69, CD117/c-kit, CD163, CD164, CD42b/GPIb alpha, CEACAM-30 1/CD66a, CEACAM-3/CD66d, CEACAM-5/CD66e, CEACAM-6/CD66c, CEACAM-8/CD66b, CXCR3, EMR1, F4/80, Fc gamma RIII (CD16), Fc gamma RIIIA/CD16a, Fc gamma RIIIB/CD16b, Flt-3/Flk-2, Glycophorin A, Glycoprotein V/CD42d, GP1BB, IL-3R alpha, Integrin alpha 2b/CD41, Integrin beta 2/CD18, Integrin beta 3/CD61, LAMP-1/CD107a, Ly-6G (Gr-1), Ly-6G/Ly-6C (Gr-1), myeloperoxidase/MPO, PEAR1, PSG1, PS G2, PSG3, PSG5, L-Selectin/CD62L, Siglec-3/CD33, thrombopoietin/Tpo, or a combination thereof.
15. The composition of statement 10-13 or 14, wherein the modifying agent is an anti-Pip4k2c antibody; a guide RNA that can bind to a Pip4k2c genomic site; a ribonucleoprotein comprising a cas nuclease and a Pip4k2c guide RNA; an inhibitory nucleic acid that can bind to an endogenous Pip4k2c nucleic acid (e.g., an endogenous Pip4k2c RNA); an expression vector encoding an inhibitory nucleic acid that can bind to an endogenous Pip4k2c nucleic acid; an antigenic Pip4k2c peptide that can induce an immune response against Pip4k2c; an expression vector that expresses an antigenic Pip4k2c peptide, the guide RNA, the cas nuclease, or a combination thereof; a small molecule inhibitor of Pip4k2c protein; a degrader of Pip4k2c protein; or a combination thereof.
16. The composition of statement 15, wherein the anti-Pip4k2c antibody is linked to a ubiquitin-protein ligase.
17. A method, comprising contacting one or more cells in vitro with the composition of any of claim 5, 10-15 or 16, and incubating one or more of the cells in a culture medium for a time and under conditions sufficient for modification of one or more of the cells to generate modified cells with reduced or eliminated expression or functioning of Pip4k2c (e.g., compared to unmodified cells).
18. The method of statement 17, wherein the cells are myeloid cells, lymphocytes, regulatory T cells, dendritic cells, bone marrow cells, granulocytes, basophils, eosinophils, neutrophils, monocytes, mast cells, erythrocytes, macrophages, platelets, tumor cells, malignant cells, or a combination thereof.
19. The method of statement 17 or 18, further comprising isolating the modified cells and administering the modified cells to a subject.
20. A method comprising administering the cells of statement 1-3 or 4, or the composition of statement 10-15 or 16 to a subject.
21. The method of statement 4, 6-8, or 19, wherein the subject is a human or a warm-blooded animal
22. The method of statement 4, 6-8 or 19, wherein the subject has cancer.
23. The method of statement 22, wherein the cancer is melanoma, intestinal cancer, breast cancer, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, hematological malignancies, Ewing's sarcoma, gestational trophoblastic tumors, hepatoblastoma, multiple myeloma, non-small cell lung cancer, retinoblastoma, or tumors in the ovaries.
24. The method of any of statements 19-23, which reduces cancer symptoms and/or the tumor loads in the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or by 100%.
25. A method comprising depleting, degrading, or inhibiting PIP4K2C in a subject.
26. The method of statement 25, which reduces the onset or severity of cancer in the subject.
27. The method of statement 25 or 26, wherein depleting, degrading, or inhibiting PIP4K2C comprises deleting or mutating a genomic site encoding a protein with at least 95% sequence identity to SEQ ID NO:1.
28. The method of statement 25, 26 or 27, wherein degrading PIP4K2C comprises contacting a binding moiety with the PIP4K2C, wherein the binding moiety is directly or indirectly linked to an agent that signals cells to degrade the PIP4K2C bound to the agent.

29. The method of statement 28, wherein the agent that signals cells to degrade the PIP4K2C bound to the agent is an E3 ubiquitin ligase.
30. The method of statement 28 or 29, wherein the binding moiety is a small molecule, an antibody, a peptide, a polysaccharide, or a lipid that binds specifically to the PIP4K2C.
31. The method of statement 28, 29, or 30, wherein the binding moiety is indirectly linked to the agent via hydrogen bonding, hydrophobic interaction, steric interaction, hydrophilic interaction, or a combination thereof.
32. The method of statement 31, wherein indirectly linked means that interaction between the binding moiety and the agent occurs before the binding moiety is contacted with the PIP4K2C.
33. The method of statement 31 or 32, wherein indirectly linked means that interaction between the binding moiety and the agent occurs after the binding moiety is contacted with the PIP4K2C.
34. The method of statement 25-32 or 33, wherein degrading the PIP4K2C comprises contacting the PIP4K2C with an antibody specific for the PIP4K2C, wherein the antibody has an Fc domain that can bind an E3 ubiquitin ligase.
35. The method of statement 25-34 or 35, wherein inhibiting the PIP4K2C comprises (a) administering an inhibitor of the PIP4K2C; or (b) modifying the pip4k2C gene sequences.
36. The method of statement 25-34 or 35, wherein inhibiting the PIP4K2C comprises inhibiting expression or function of the PIP4K2C.
37. The method of statement 25-35 or 36, wherein inhibiting expression or function of the PIP4K2C comprises administering a binding moiety, antibody, nucleic acid inhibitor, or small molecule inhibitor of the PIP4K2C.
38. The method of statement 25-36 or 37, wherein the subject has or is suspected of having cancer, immune deficiency, autoimmune disease, infection, or a combination thereof.
39. The method of statement 28-37 or 38, wherein the binding moiety binds with specificity to the PIP4K2C protein.
40. The method of statement 28-38 or 39, wherein the binding moiety binds with specificity to an epitope having sequence with at least 95% sequence identity to a 5-amino acid to 30 amino acid portions of SEQ ID NO:1.
41. The method of statement 25-39 or 40, wherein inhibiting the PIP4K2C comprises inhibiting expression of the PIP4K2C by contacting a nucleic acid encoding the PIP4K2C with a small hairpin RNA, an siRNA, or a vector that can express a small hairpin RNA or an siRNA.
42. The method of statement 41, wherein the small hairpin RNA, the siRNA, or a combination thereof binds to an RNA with at least 95% sequence identity or complementarity to SEQ ID NO:2.
43. The method of statement 25-41 or 42, wherein inhibiting the PIP4K2C comprises Cre/lox-mediated, floxing (flox/flox)-mediated, CRISPR-mediated, TALENS-mediated, or ZFN-mediated knockout or knockdown of pip4k2c.
44. The method of statement 25-42 or 43, comprising isolating a population of cells from the subject and incubating the cells with one or more CRISPR,

TALENS, Cre/lox, or ZFN reagents to generate a modified population of cells with one or more modified pip4k2c gene sequences.
45. The method of statement 43 or 44, wherein the one or more CRISPR, TALENS, or ZFN reagents comprises one or more guide RNAs or a vector that can express one or more guide RNAs, where the one or more of the guide RNAs can specifically bind to a PIP4K2C genomic site.
46. The method of any of statements 25-45, which reduces cancer symptoms and/or the tumor loads in the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or by 100%.
47. A kit comprising one or more agents that can modify, degrade, modulate, or inhibit a PIP4K2C protein or a Pip4k2c nucleic acid, and instructions for using 20 one or more of the agents.
48. The kit of statement 47, wherein the one or more agents is directly or indirectly linked to one or more binding moieties that specifically binds to at least one PIP4K2C.
49. The kit of statement 47 or 48, wherein one or more of the agents is one or more binding moieties, each binding moiety directly or indirectly linked to an agent that signals cells to degrade the PIP4K2C when the binding moiety is bound to PIP4K2C.
50. The kit of statement 49, wherein the agent that signals cells to degrade the PIP4K2C is an E3 ubiquitin ligase.
51. The kit of statement 47-49 or 50, further comprising the agent that signals cells to degrade the PIP4K2C.
52. The kit of statement 47-50, or 51, wherein the binding moiety is a small molecule, an antibody, a peptide, a polysaccharide, or a lipid that binds specifically to one of the PIP4K2C.
53. The kit of statement 47-51 or 52, wherein the binding moiety is indirectly linked to the agent via hydrogen bonding, hydrophobic interaction, steric interaction, hydrophilic interaction, or a combination thereof.
54. The kit of statement 48-52 or 53, wherein indirectly linked means that interaction between the binding moiety and the agent occurs before the binding moiety is contacted with one of the PIP4K2C.
55. The kit of statement 48-53 or 54, wherein indirectly linked means that interaction between the binding moiety and the agent occurs after the binding moiety is contacted with one of the PIP4K2C.
56. The kit of statement 47-54 or 55, wherein degrade or degrading comprises contacting one or more the PIP4K2C with an antibody specific for the PIP4K2C, wherein the antibody has an Fc domain that can bind an E3 ubiquitin ligase.
57. A kit comprising components that include one or more sterile implements for isolating cells from a subject, reagents for culturing cells, one or more guide RNA(s) for targeting one or more genomic pip4k2c sites, implements for administering modified cells back into the subject, and any combination thereof.
58. The kit of statement 57, further comprising instructions for using the components to modify genomic pip4k2c sites and thereby inhibit PIP4K2C activity in the subject.
59. A method comprising knockdown or knockout of PIP4K2C in a population of mammalian cells to generate a population of modified mammalian cells with reduced expression or function of the PIP4K2C.
60. The method of statement 59, further comprising administering the population of modified mammalian cells to a subject.
61. The method of statement 59 or 60, wherein the population of modified mammalian cells is allogenic or autologous to the subject.
62. Thr method of statement 59, 60, or 61, wherein the mammalian cells comprise myeloid cells, lymphocytes, regulatory T cells, dendritic cells, bone marrow cells, granulocytes, basophils, eosinophils, neutrophils, monocytes, mast cells, erythrocytes, macrophages, platelets, tumor cells, malignant cells or a combination thereof.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A population of modified cells comprising knockdown or knockout of the cells' endogenous pip4k2c, wherein the modified cells comprise myeloid cells, lymphocytes, regulatory T cells, dendritic cells, bone marrow cells, granulocytes, basophils, eosinophils, neutrophils, monocytes, mast cells, erythrocytes, macrophages, platelets, tumor cells, malignant cells or a combination thereof.

2. A method comprising administering the population of modified cells of claim 1 to a subject.

3. The method of claim 2, wherein the subject has cancer or is suspected of developing cancer.

4. The method of claim 2, wherein the population of modified cells are administered in a therapeutically effective amount.

5. The method of claim 4, wherein the therapeutically effective amount reduces cancer symptoms and/or the tumor loads in the subject by at least 20%.

6. A composition comprising one or more agents that can modify, degrade, or inhibit Pip4k2c protein or a pip4k2c nucleic acid.

7. The composition of claim 6, wherein the one or more agents comprise anti-Pip4k2c antibodies, Pip4k2c nucleic acid inhibitors, Pip4k2c degraders, Pip4k2c vaccines, small molecule Pip4k2c inhibitors, Pip4k2c guide RNAs with cas nucleases, and combinations thereof.

8. A kit comprising the composition of claim 6 and instructions for using the composition.

9. The kit of claim 8, comprising one or more sterile implements for isolating cells from a subject, one or more reagents for culturing cells, one or more guide RNA(s) for targeting one or more genomic pip4k2c sites, one or more implements for administering modified cells back into the subject, and any combination thereof.

10. The kit of claim 8, further comprising instructions for using the implements and/or reagents to modify genomic pip4k2c sites and thereby inhibit PIP4K2C activity.

11. A method comprising depleting, degrading, or inhibiting PIP4K2C in one or more cells of a subject.

12. The method of claim 11, wherein depleting, degrading, or inhibiting PIP4K2C comprises deleting, degrading, or mutating a genomic site encoding a protein with at least 95% sequence identity to SEQ ID NO:1.

13. The method of claim 12, wherein degrading PIP4K2C comprises contacting PIP4K2C with a degrader compound.

14. The method of claim 12, wherein degrading PIP4K2C comprises contacting a binding moiety with the PIP4K2C, wherein the binding moiety is directly or indirectly linked to an agent that signals cells to degrade the PIP4K2C bound to the agent.

15. The method of claim 14, wherein the agent that signals cells to degrade the PIP4K2C is an E3 ubiquitin ligase.

16. The method of claim 14, wherein the binding moiety is a small molecule, an antibody, a peptide, a polysaccharide, or a lipid that binds specifically to the PIP4K2C.

17. The method of claim 14, wherein the binding moiety is indirectly linked to the agent via hydrogen bonding, hydrophobic interaction, steric interaction, hydrophilic interaction, or a combination thereof.

18. The method of claim 14, wherein indirectly linked means that interaction between the binding moiety and the agent occurs before the binding moiety is contacted with the PIP4K2C.

19. The method of claim 17, wherein indirectly linked means that interaction between the binding moiety and the agent occurs after the binding moiety is contacted with the PIP4K2C.

20. The method of claim 11, wherein degrading the PIP4K2C comprises contacting the PIP4K2C with an antibody specific for the PIP4K2C, wherein the antibody has an Fc domain that can bind an E3 ubiquitin ligase.

21. The method of claim 11, wherein inhibiting the PIP4K2C comprises inhibiting expression or function of the PIP4K2C.

22. The method of claim 11, wherein inhibiting the PIP4K2C comprises (a) administering an inhibitor of the PIP4K2C; or (b) modifying or inhibiting expression of pip4k2C gene sequences.

23. The method of claim 21, wherein inhibiting expression or function of the PIP4K2C comprises administering a binding moiety, antibody, nucleic acid inhibitor, or small molecule inhibitor of the PIP4K2C.

24. The method of claim 23, wherein the binding moiety binds with specificity to the PIP4K2C protein.

25. The method of claim 23, wherein the binding moiety binds with specificity to an epitope having sequence with at least 95% sequence identity to a 5-amino acid to 30 amino acid portion of SEQ ID NO:1.

26. The method of claim 21, wherein inhibiting expression or function comprises contacting a nucleic acid encoding the PIP4K2C with a small hairpin RNA, an siRNA, or a vector that can express a small hairpin RNA or an siRNA.

27. The method of claim 26, wherein the small hairpin RNA, the siRNA, or a combination thereof binds to an RNA with at least 95% sequence identity or complementarity to SEQ ID NO:2.

28. The method of claim 11, wherein inhibiting the PIP4K2C comprises Cre/lox-mediated, floxing (flox/flox)-mediated, CRISPR-mediated, TALENS-mediated, or ZFN-mediated knockout or knockdown of a pip4k2c gene in one or more cells of a subject.

29. The method of claim 11, comprising isolating one or more cells from the subject and incubating the cells with one or more CRISPR, TALENS, Cre/lox, or ZFN reagents to generate a modified population of cells with one or more modified pip4k2c gene sequences.

30. The method of claim 29, wherein the one or more CRISPR, TALENS, or ZFN reagents comprises one or more guide RNAs or a vector that can express one or more guide RNAs, where the one or more of the guide RNAs can specifically bind to a PIP4K2C genomic site.

31. The method of claim 11, performed in vitro.

32. The method of claim 11, performed in vivo.

33. The method of claim 11, wherein the subject has or is suspected of having cancer, immune deficiency, autoimmune disease, infection, or a combination thereof.

34. The method of claim 33, which reduces the onset or severity of cancer in the subject.

35. The method of claim 11, which reduces cancer symptoms and/or the tumor loads in the subject by at least 20%.
